(19) United States
(12) Patent Application Publication
(10) Pub. No.: US 2021/0110925 A1
(43) Pub. Date: Apr. 15, 2021

(54) METHOD, MODULE AND SYSTEM FOR ANALYSIS OF BRAIN ELECTRICAL ACTIVITY

(71) Applicants: Adaptive, Intelligent and Dynamic Brian Corporation (AidBrain), Taoyuan (TW); NORDEN E. HUANG, Bethesda, MD (US)

(72) Inventor: NORDEN E. HUANG, Bethesda, MD (US)

(21) Appl. No.: 16/608,192

(22) PCT Filed: May 22, 2018
(86) PCT No.: PCT/US2018/033769

§ 371 Date: Oct. 25, 2019

Related U.S. Application Data
(60) Provisional application No. 62/509,199, filed on May 22, 2017.

(51) Int. Cl. G16H 50/20; A61B 5/00; A61B 5/374; A61B 5/384; A61B 5/37; A61B 5/245

(52) U.S. Cl. CPC G16H 50/20; A61B 5/742; A61B 5/374; A61B 5/245; A61B 5/37; A61B 5/7253; A61B 5/384

(57) ABSTRACT

The present disclosure provides a system for analyzing electrical activities of at least one brain. The system comprises a visual output module for rendering a visual output space according to analyzed data sets generated by an analysis module, and displaying a visual output. The visual output comprises a first axis representing FM, a second axis representing AM, and a plurality of visual elements defined by the first axis and the second axis. Each of the visual elements comprises an accumulated signal strength and the analyzed data sets. Each of the analyzed data sets comprises a plurality of analyzed data units collected over a time period.

### CROSS-REFERENCE TO RELATED APPLICATION

The present disclosure claims priority to U.S. provisional patent application No. 62/509,199, filed on May 22, 2017, the entirety of which is incorporated herein by reference.

### FIELD

The present disclosure is generally related to analysis of physiological signals. More particularly, the present disclosure is related to analysis of electrical activities of the brain.

### BACKGROUND

The brain function is dynamic and relevant to brain structures and electrical activities of the bran. Structural deficiencies of the brain could be detected by various conventional medical imaging techniques such as computed tomography (CT) scan, magnetic resonance imaging (MRI), positron emission tomography (PET), and single-photo emission computed tomography (SPET). However, the conventional medical imaging techniques could not capture the dynamic nature of the brain functions. Furthermore, many mental or psychiatric conditions have no discernable structural changes in the brain, these conditions may include: depression, insomnia, mild cognitive impairment, the initial stage of Alzheimer's disease, ADHD, and different depths of anesthesia.

Electroencephalography (EEG), magnetoencephalography (MEG), and electrocorticography (ECoG) can be used to measure the electrical activities of the brain, these methods provide real-time information of the brain function that are important in diagnosis, prognosis, staging, or clinical evaluation on certain neurological diseases. While ECoG requires a craniotomy and is an invasive procedure, EEG and MEG are non-invasive and in-expensive approaches to monitor the electrical activities of the brain. However, given the non-invasive nature of EEG and MEG, they can be interfered or disturbed by various anatomical structures of the head or the brain, such as conductivity variations of the scalp (skull compacta and skull spongiosa), cerebral spinal fluid (CSF), gray matter, and white matter. On other hand, ECoG is less disturbed and interfered by the anatomical structures of the head, because ECoG places detection modules directly on an exposed surface of the brain to measure the electrical activities.

Additionally, the non-stationary and non-linear nature of electrical activities of the brain are significant obstacles for signal processing. Conventional approaches for signal processing of EEG, MEG, or ECoG signals have failed to provide an effective solution to the obstacles. For instance, Fourier transformation are often used to interpret linear and stationary wave signals, such as spectrum analysis; however, due to its mathematical nature and probability distribution, Fourier transformation is unable to provide meaningful visualization results from non-stationary and non-linear wave signals.

The Holo-Hilbert spectral analysis (HOSA) is a tool for visualizing non-stationary and non-linear waves. The mathematics behind HOSA has been summarized in Huang et al (Huang, N. E., Hu, K., Yang, A. C., Chang, H. C., Jia, D., Liang, W. K., Yeh, J. R., Kao, C. L., Juan, C. H., Peng, C. K. and Meijer, J. H. (2016). On Holo-Hilbert spectral analysis: a full informational spectral representation for nonlinear and non-stationary data. Phil. Trans. R. Soc. A, 374(2065)). HOSA adopts some of the mathematical methodologies of Hilbert-Huang transformation when analyzing non-stationary and non-linear waves. However, the application of HOSA on analysis of brain signals has never been explored and exploited.

Due to the lack of adequate signal processing tools, data associated with electrical activities of the brain often needs to be analyzed by trained professionals, in addition to available algorithms or software embedded instruments. For instance, EEG data could be massive in terms of their quantity and complexity, because EEG signals are generated from several EEG leads on the head and can be disturbed or interfered by anatomical structures on the head.

Given the non-linear and non-stationary nature and the inherent complexity and quantity of electrical activities of the brain, there is a need for an efficient and intuitive mean for analysis and visualization of EEG and MEG.

### BRIEF DESCRIPTION OF THE DRAWINGS

It is an object of the present disclosure to provide HOSA-based methods and systems for analysis of electrical activities of the brain.

It is an object of the present disclosure to provide one or more visual outputs of electroencephalography (EEG), magnetoencephalography (MEG), and electrocorticography (ECoG).

It is also an object of the present disclosure to provide methods or systems for presenting one or more amplitude-versus-time graphs of EEG, MEG, and ECoG signals.

It is also an object of the present disclosure to provide one or more visual outputs of abnormal EEG, MEG, and ECoG signals.

It is also an object of the present disclosure to provide one or more visual outputs to compare electrical activities of the brain in different groups of subjects, different subjects, or different time periods of the same subjects.

It is also an object of the present disclosure to provide applications of HOSA in diagnosis of neurological disorders.

An embodiment of the present disclosure provides a non-transitory computer program product embodied in a computer-readable medium. When the non-transitory computer program product is executed by one or more analysis modules, the non-transitory computer program product provides a visual output for presenting electrical activities of at least one brain. The non-transitory computer program product comprises a first axis representing frequency modulation (FM), a second axis representing amplitude modulation (AM); and a plurality of visual elements, each of the visual elements being defined by the first axis and the second axis, and each of the visual elements comprising an accumulated signal strength and a plurality of analyzed data units collected over a time period. Each of the analyzed data units comprises a first coordinate, a second coordinate, and a signal strength value. The first coordinate is an argument of a FM function from a transformation on a primary intrinsic mode function (IMF). The second coordinate is an argument of an AM function from a transformation on a secondary IMF. Each of the primary IMF is generated from an empirical mode decomposition (EMD) of a plurality of electrical activity signals, and each of the secondary IMF is generated from an EMD of the primary IMF, and the accumulated signal strength is an integral of the strength values of the analyzed data units.

In a preferred embodiment, the first axis is a logarithmic scale of FM, the second axis is a logarithmic scale of AM, the first coordinate is a logarithmic value of the argument of the FM function, and the second coordinate is a logarithmic value of the argument of the AM function.

In a preferred embodiment, the analyzed data units are generated from EEG, MEG, or ECoG.

In a preferred embodiment, the visual elements are functional electroencephalotopography (the fEEToPG) or functional electroencephalotomography (the fEEToMG).

In a preferred embodiment, each of the visual elements further comprises a boundary defining an anatomical graph, and the anatomical graph is a two-dimensional graph of the brain when the visual elements are fEEToPG.

In a preferred embodiment, each of the visual elements further comprises a boundary defining an anatomical graph, and the anatomical graph is a three-dimensional graph of the brain when the visual elements are fEEToMG.

In a preferred embodiment, the visual element further comprises one or more detection unit IDs in the boundary, and each of the detection unit IDs has one of the accumulated signal strengths.

In a preferred embodiment, the visual element further comprises a plurality of intermediate areas within the boundary and between the detection unit IDs, and each of the intermediate areas has a modeled accumulated signal strength.

In a preferred embodiment, the accumulated signal strength in each of the visual elements is indicated by different colors, grayscales, dot densities, contour lines, or screentones.

An embodiment of the present disclosure provides a non-transitory computer program product embodied in a computer-readable medium. When the non-transitory computer program product is executed by one or more analysis modules, the non-transitory computer program provides statistical significance between at least two visual outputs. The non-transitory computer program products comprise a first axis representing FM, a second axis representing AM, and a plurality of visual elements. Each of the visual elements are defined by the first axis and the second axis, and each of the visual elements comprises a probability for quantifying the statistical significance between other visual outputs. Each of the visual outputs comprises a plurality of analyzed data units collected over a time period. Each of the analyzed data units comprises a first coordinate, a second coordinate, and a signal strength value. The first coordinate is an argument of a FM function from a transformation on a primary IMF. The second coordinate is an argument of an AM function from a transformation on a secondary IMF. Each of the primary IMF is generated from an EMD of a plurality of electrical activity signals, and each of the secondary IMF is generated from an EMD of the primary IMF. The accumulated signal strength is an integral of the signal strength values of the analyzed data units.

In a preferred embodiment, the probability for quantifying the statistical significance is a P-value.

An embodiment of the present disclosure provides a system for analyzing electrical activities of at least one brain. The system comprises a detection module for detecting the electrical activities of the brain, a transmission module for receiving electrical activity signals from the detection module and delivering the electrical activity signals to the analysis module, an analysis module for generating a plurality of analyzed data sets from the electrical activity signals, and a visual output module for rendering a visual output space according to the analyzed data sets generated by the analysis module, and displaying a visual output. The visual output comprises a first axis representing FM, a second axis representing AM, and a plurality of visual elements defined by the first axis and the second axis. Each of the visual elements comprises an accumulated signal strength and the analyzed data sets. Each of the analyzed data sets comprises a plurality of analyzed data units collected over a time period. Each of the analyzed data units comprises a first coordinate, a second coordinate, and a signal strength value, the first coordinate is an argument of a FM function and the second coordinate is an argument of an AM function, and the accumulated signal strength is an integral of the signal strength value of the analyzed data units.

In a preferred embodiment, the system further comprises a non-transitory computer program product for presenting the electrical activities of the brain, wherein the program product comprises set of instructions that, when executed by the analysis module, causes the analysis module to perform actions comprising: 1) performing EMD on the electrical activity signals to generate a set of IMFs; 2) performing EMD on the set of primary IMFs to generate a set of secondary IMFs; 3) performing transformations on the set of primary IMFs to generate FM functions and on the set of secondary IMFs to generate AM functions; and 4) combining the AM functions and the FM functions to generate a plurality of analyzed data sets.

An embodiment of the present disclosure provides a system for analyzing electrical activities of at least one brain. The system comprises an analysis module for generating a set of probabilities for quantifying statistical significance between at least two visual outputs. Each of the visual outputs comprises a plurality of analyzed data units collected over a time period, each of the analyzed data units comprises a first coordinate, a second coordinate, and a signal strength value. The first coordinate is an argument of FM function from a transformation on a primary IMF. The second coordinate is an argument of an AM function form a transformation on a secondary IMF. Each of the primary IMF is generated from an EMD of a plurality of electrical activity signals, and each of the secondary IMF is generated from an EMD of the primary IMF, and accumulated signal strength is an integral of the signal strength values of the analyzed data units. The system further comprises a visual output module for rendering a visual output space according to the set of probabilities, and displaying a visual output, wherein the visual output comprises a first axis representing FM, a second axis representing AM, and a plurality of visual elements defined by the first axis and the second axis. Each of the visual elements comprises a probability for quantifying the statistical significance between other visual outputs.

An embodiment of the present disclosure provides a method for analyzing electrical activities of at least one brain, comprising: 1) detecting the electrical activities of the brain by a detection module; 2) performing EMD on electrical activity signals to generate a set of IMFs; 3) performing EMD on the set of primary IMFs to generate a set of secondary IMFs; 4) performing transformations on the set of primary IMFs to generate FM functions and on the set of secondary IMFs to generate AM functions; 5) combining the AM functions and the FM functions to generate a plurality of analyzed data sets, and each of the analyzed data sets comprising a plurality of analyzed data units collected over a time period; 6) rendering a visual output space according to the analyzed data sets; and 7) displaying a visual output comprising a first axis representing FM, a second axis representing AM, and a plurality of visual elements defined by the first axis and the second axis, and each of the visual elements comprising an accumulated signal strength and the analyzed data sets. The accumulated strength is an integral of the signal strength value of each of the analyzed data units.

### BRIEF DESCRIPTION OF THE DRAWINGS

Implementations of the present technology will now be described, by way of examples only, with reference to the attached figures.

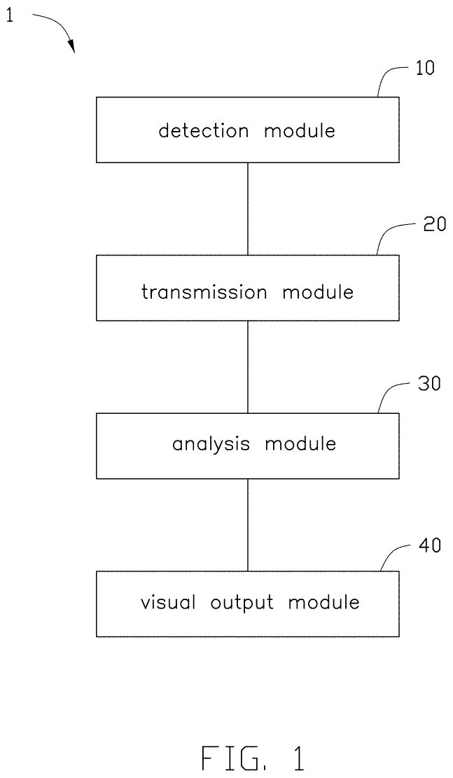

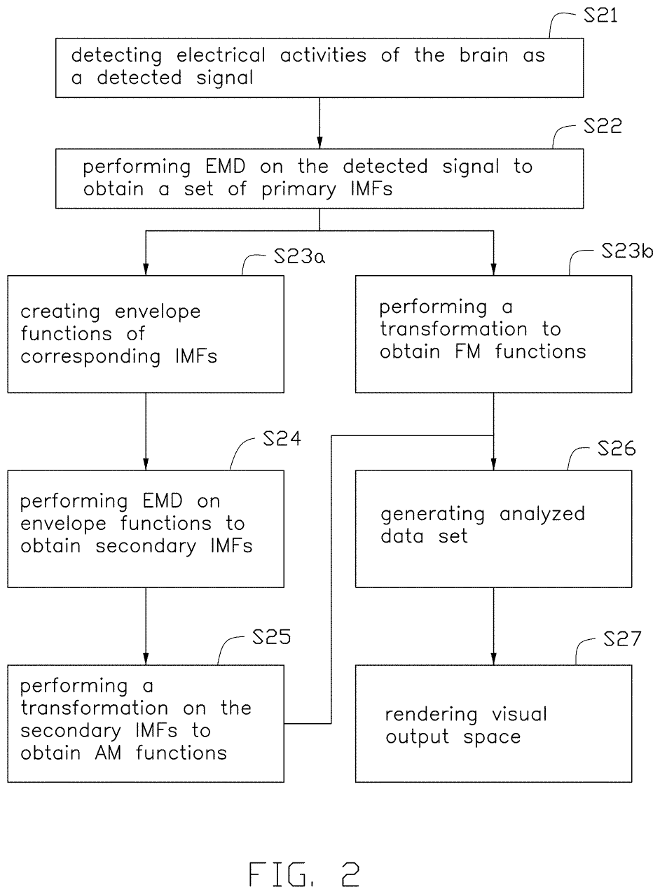

FIG. 1 is a schematic diagram of a system for analyzing electrical activities of a brain in accordance with an embodiment of the present disclosure.

FIG. 2 is a flow diagram of a method for analyzing electrical activities of the brain in accordance with an embodiment of the present disclosure.

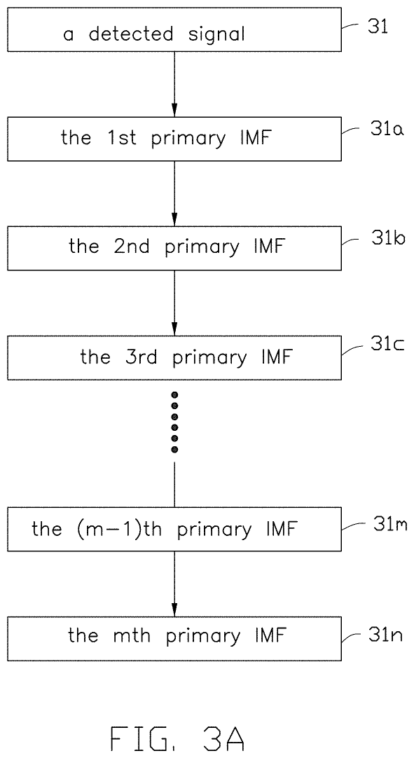

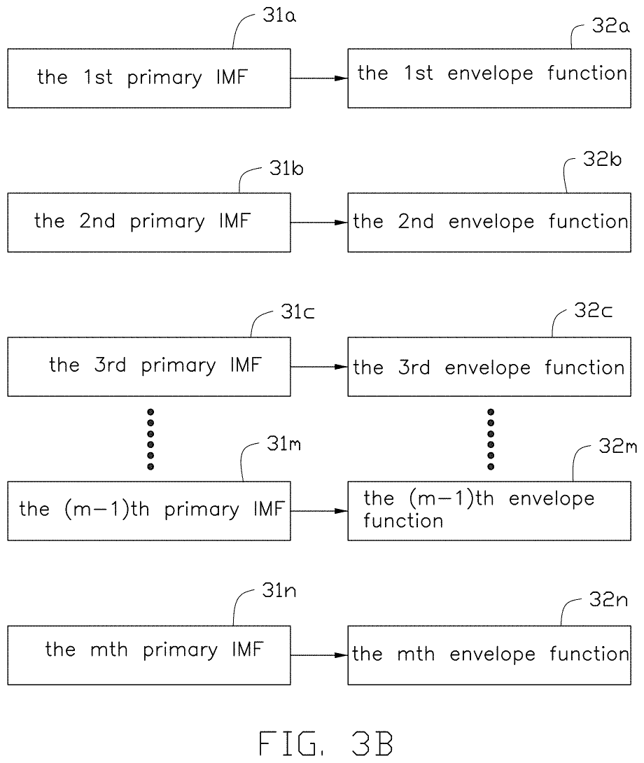

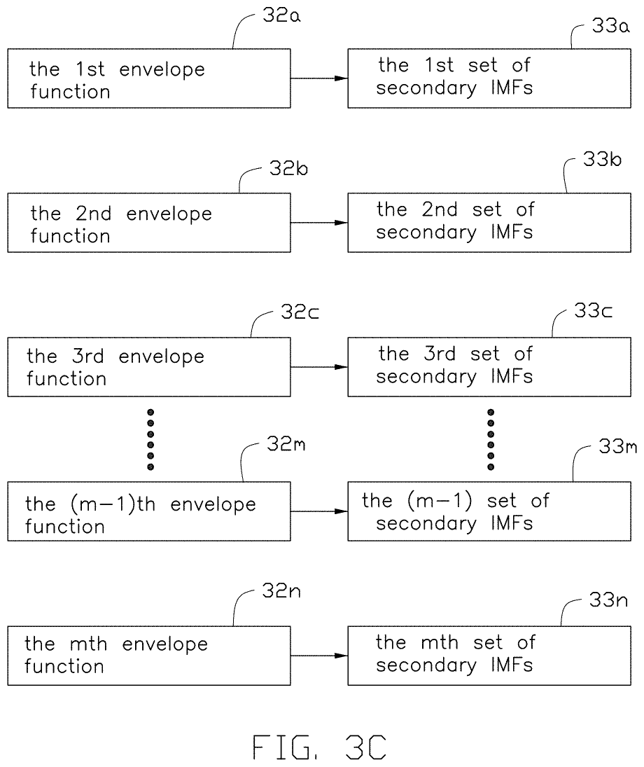

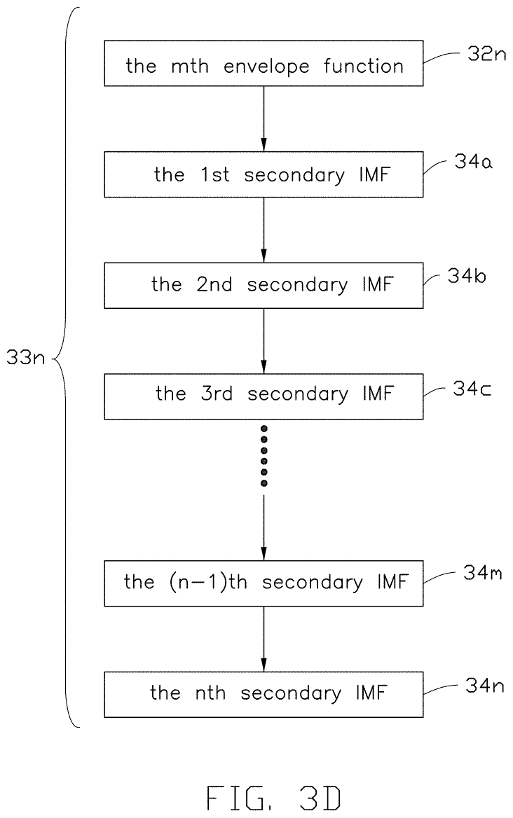

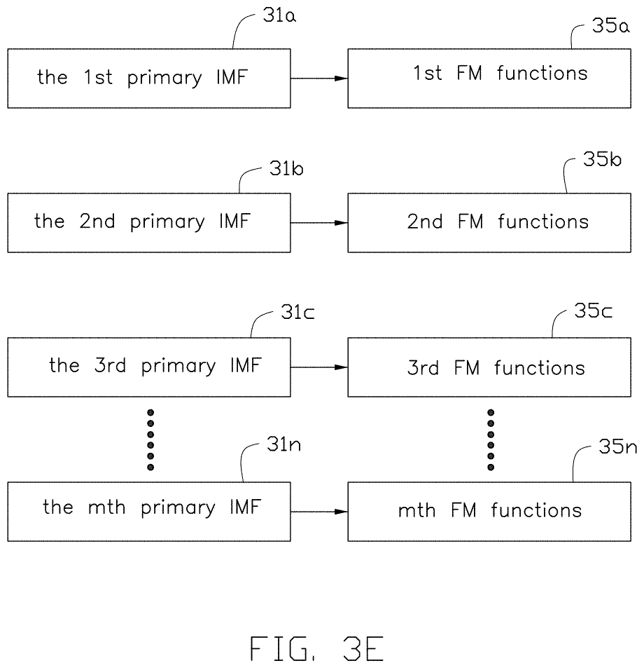

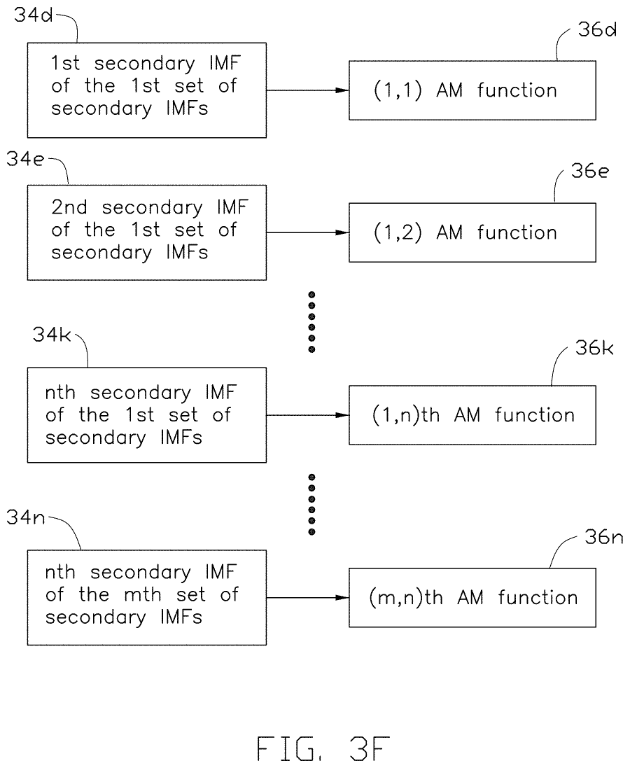

FIG. 3A is a flow diagram of transforming electrical activity signals into a set of primary intrinsic mode functions (IMFs); FIG. 3B is a flow diagram of an interpolation process; FIG. 3C is a flow diagram of empirical mode decomposition (EMD); FIG. 3D is a flow diagram of secondary IMFs generated from envelope functions; FIG. 3E is a flow diagram of transforming primary IMFs into frequency modulation (FM) functions; and FIG. 3F is a flow diagram of transforming secondary IMFs into amplitude modulation (AM) functions, in accordance with embodiments of the present disclosure.

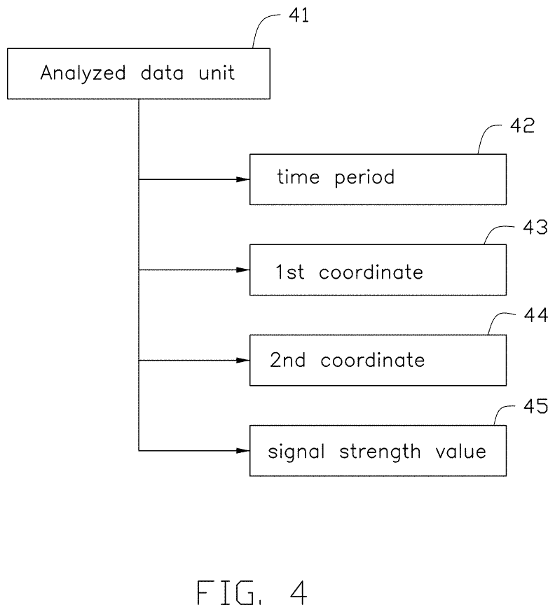

FIG. 4 is a schematic diagram of an analyzed data unit in accordance with an embodiment of the present disclosure.

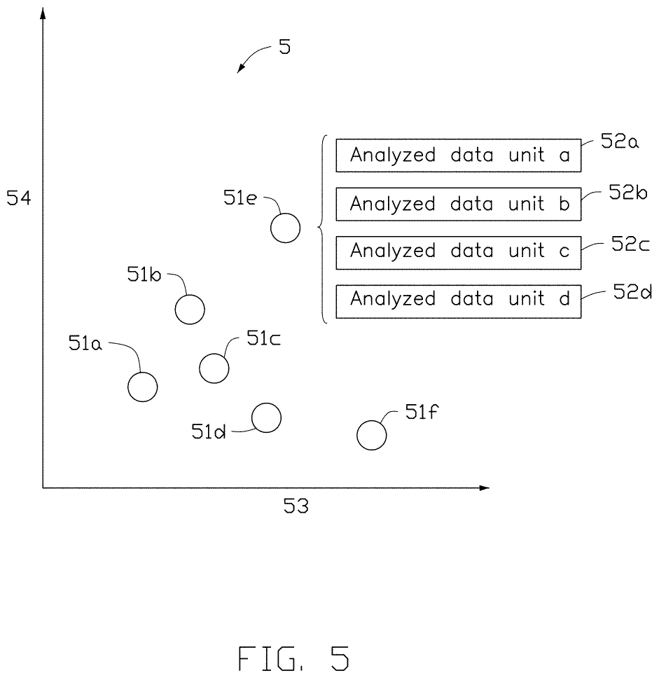

FIG. 5 is a schematic illustration of a visual output of a plurality of analyzed data sets in accordance with embodiments of the present disclosure.

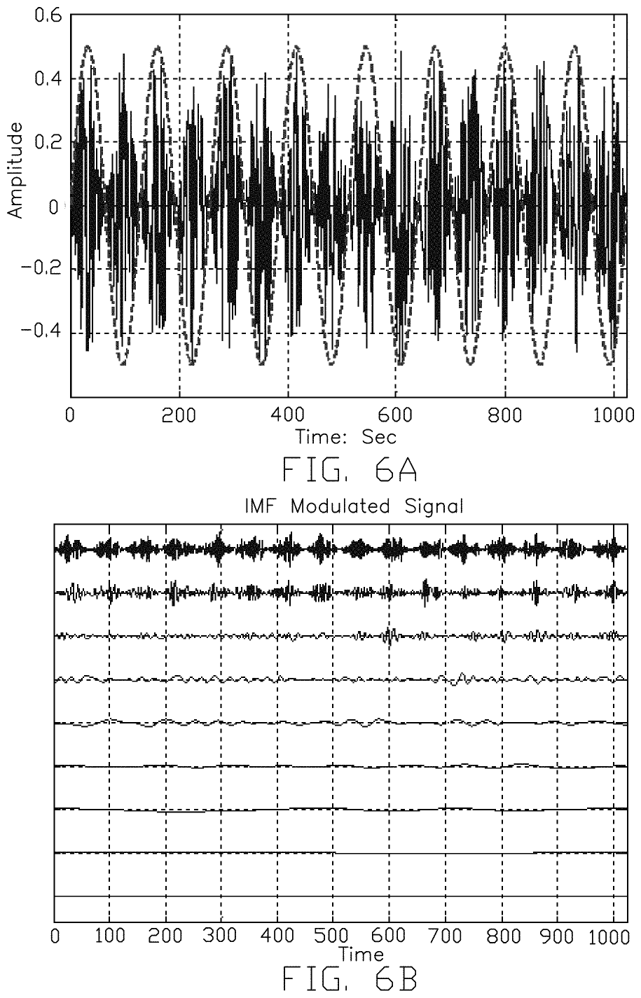

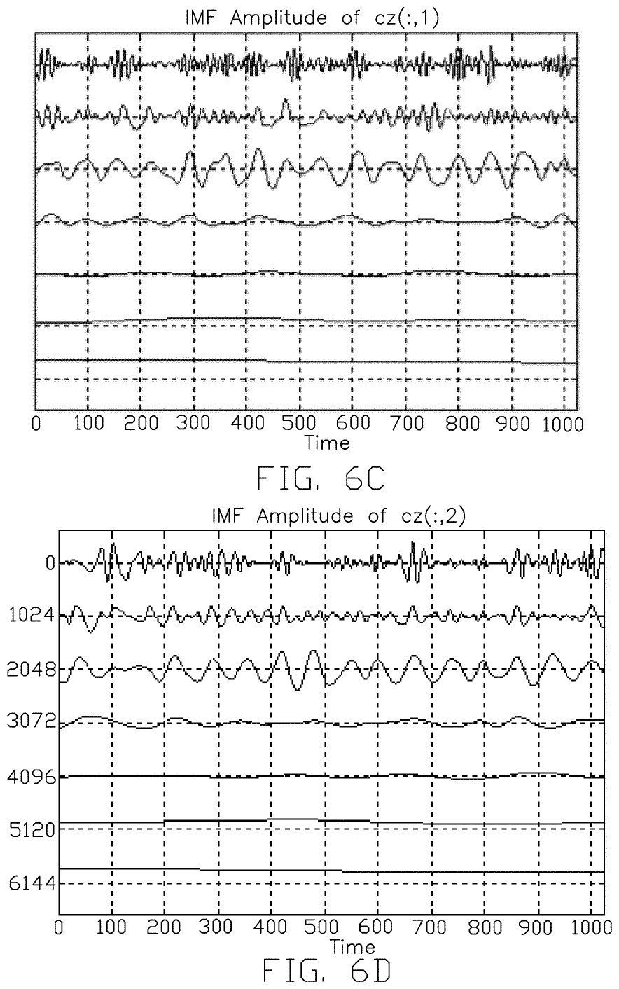

FIG. 6A is a marked-up amplitude-versus-time graph for the electrical activity signals; FIG. 6B, FIG. 6C, and FIG. 6D are IMF modulated signal graphs in accordance with embodiments of the present disclosure.

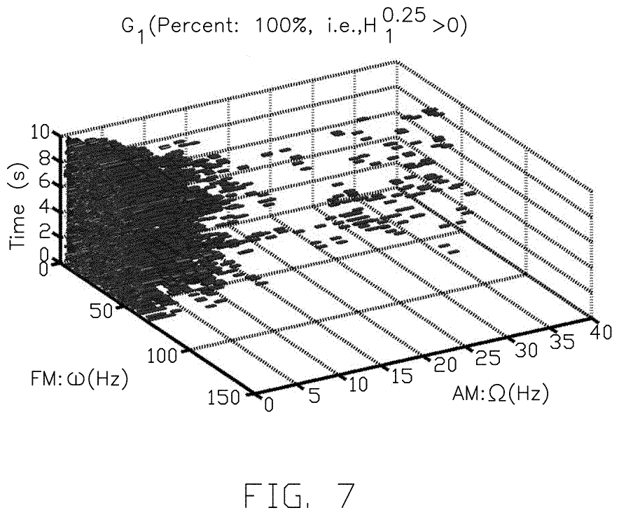

FIG. 7 is a plot graph for a plurality of analyzed data sets in accordance with an embodiment of the present disclosure.

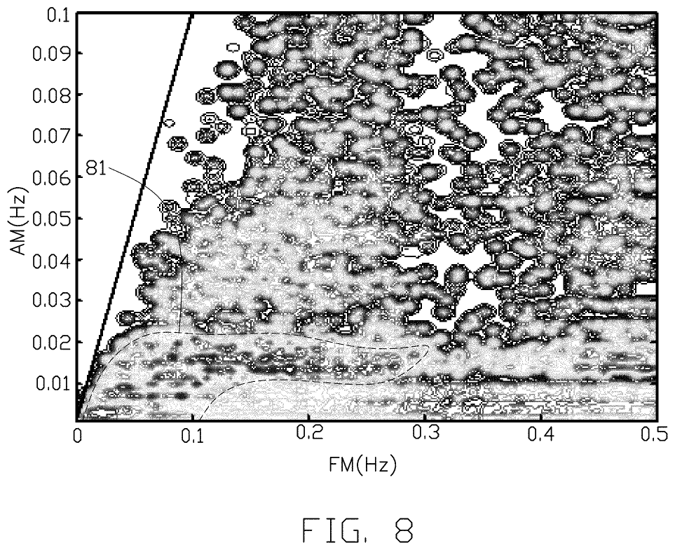

FIG. 8 is a marked-up heat map transformed from the plot graph of FIG. 7 in accordance with an embodiment of the present disclosure.

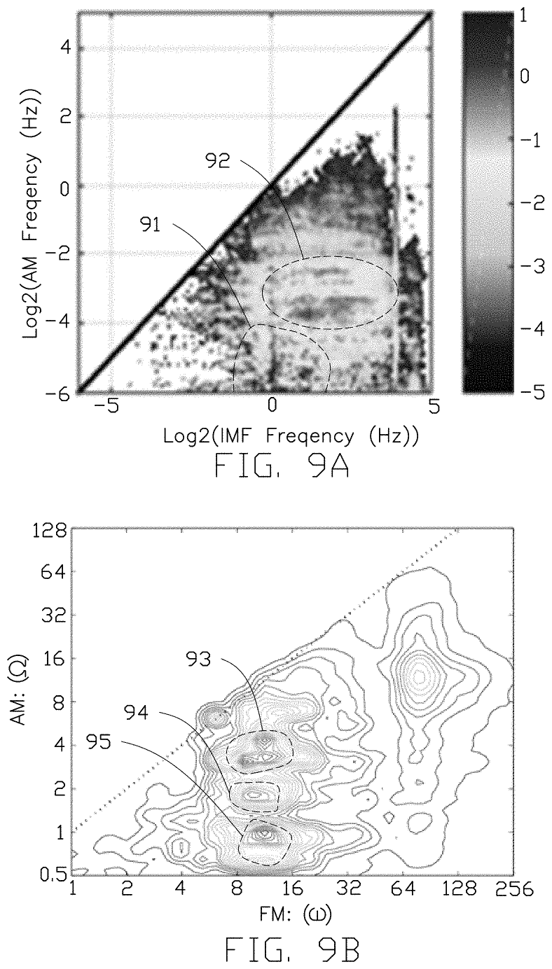

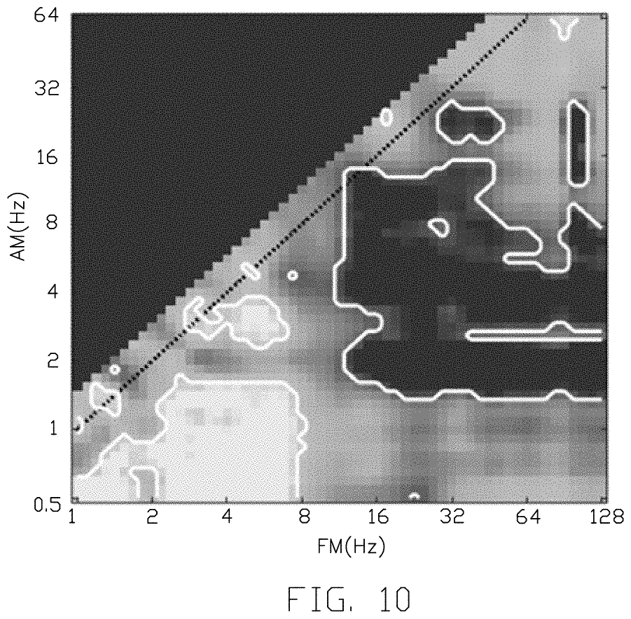

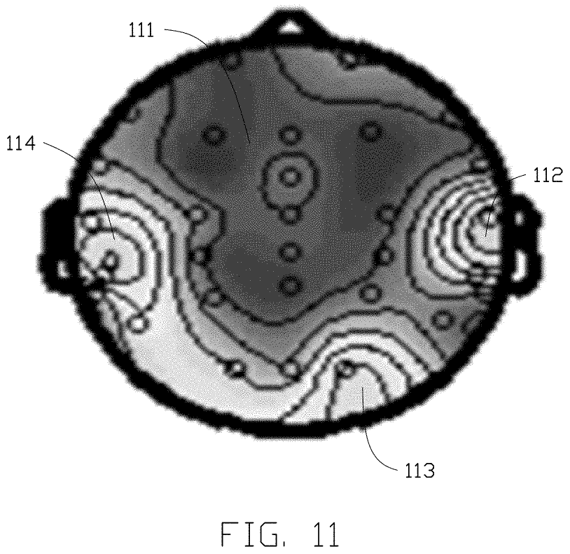

FIG. 9A and FIG. 9B are marked-up visual outputs of the analyzed data sets in accordance with embodiments of the present disclosure.

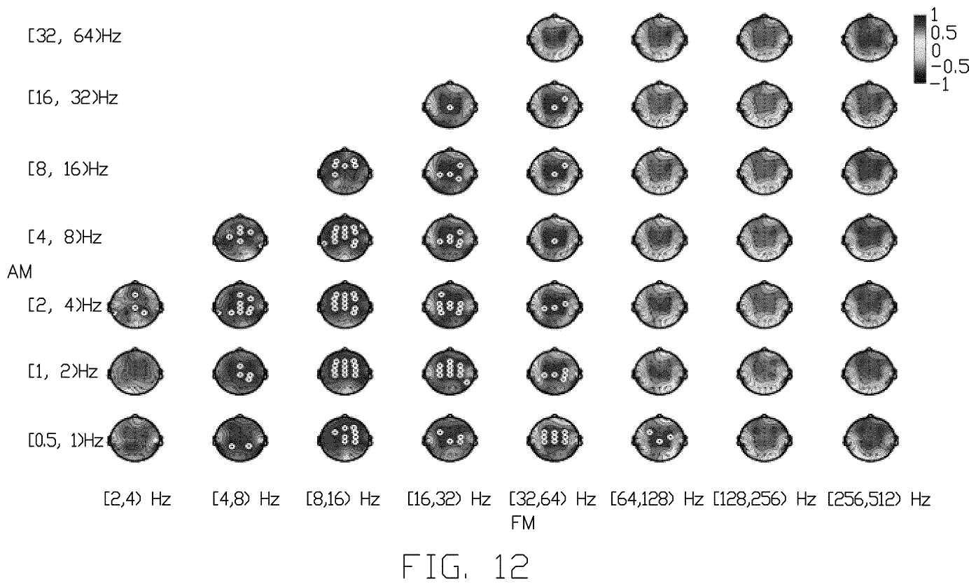

FIG. 10 is a marked-up visual output with enhanced contrast of the analyzed data sets in accordance with embodiments of the present disclosure.

FIG. 11 is a visual output of functional electroencephalotopography (the fEEToPG) comprising a brain topography in accordance with an embodiment of the present disclosure.

FIG. 12 is a visual output of the fEEToPG comprising a plurality of brain topographies in accordance with embodiments of the present disclosure.

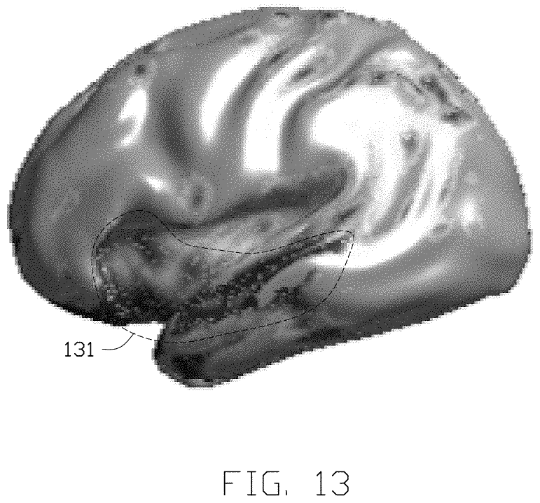

FIG. 13 is a marked-up visual output of electroencephalotomography (the fEEToMG) comprising a brain tomography in accordance with an embodiment of the present disclosure.

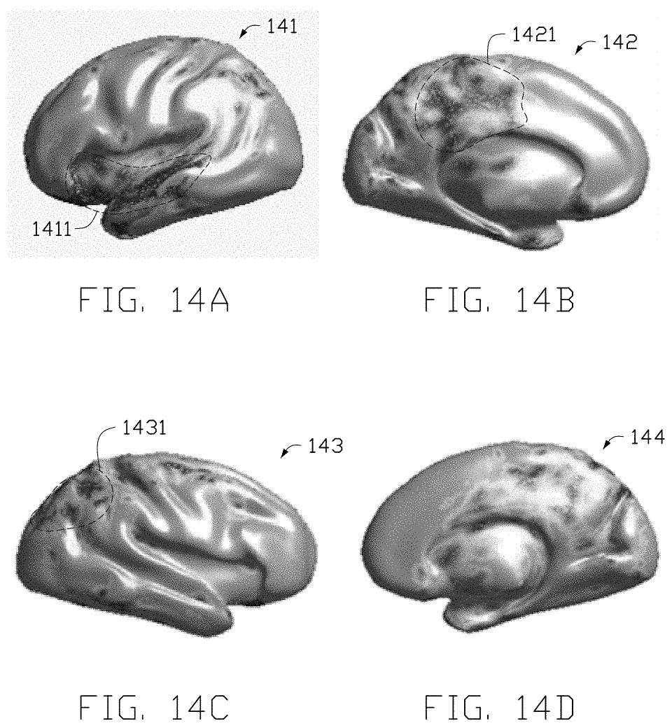

FIG. 14A, FIG. 14B, and FIG. 14C are marked-up visual outputs of the fEEToMG, and FIG. 14D is a marked-up visual output of the fEEToMG with embodiments of the present disclosure.

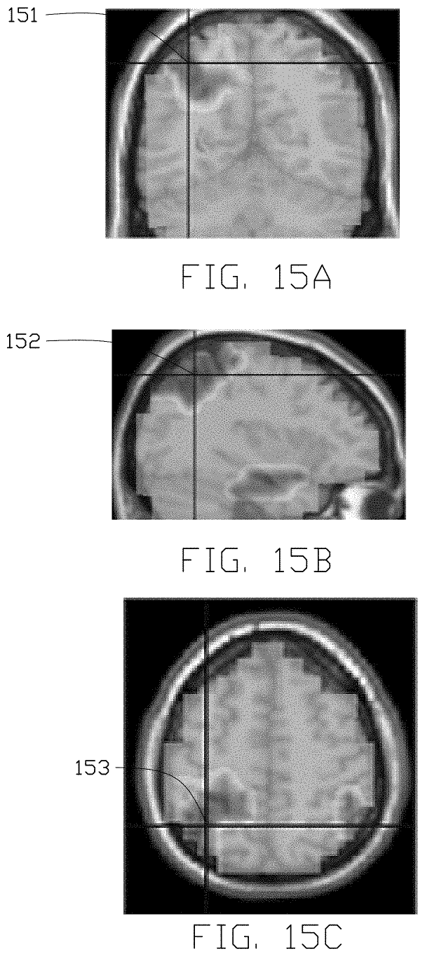

FIG. 15A, FIG. 15B, and FIG. 15C are marked-up MRI images modeled with tomographies generated by the HOSA method in accordance with embodiments of the present disclosure.

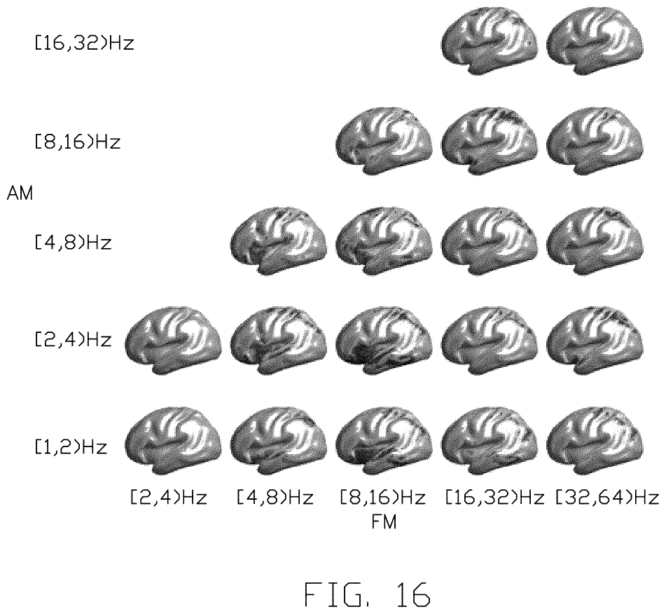

FIG. 16 is a visual output of the fEEToMG comprising a plurality of brain tomographies in accordance with embodiments of the present disclosure.

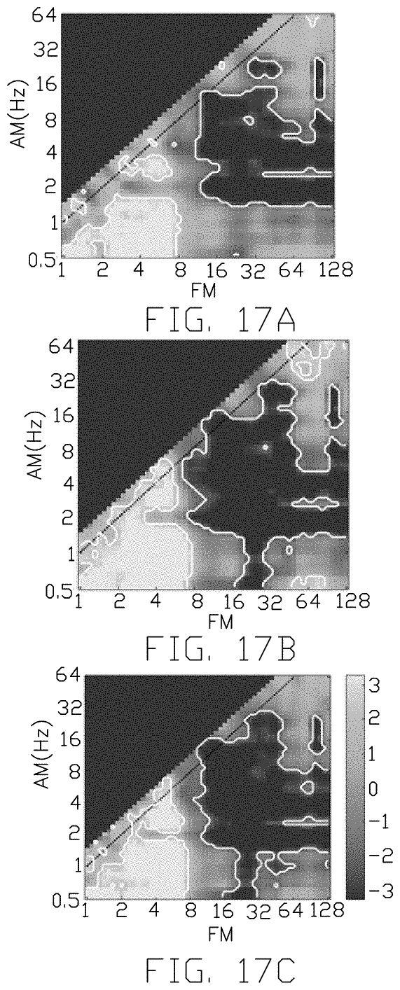

FIG. 17A, FIG. 17B, and FIG. 17C are marked-up visual outputs of the fEEGs for detection or diagnosis of dementia, in accordance with embodiments of the present disclosure.

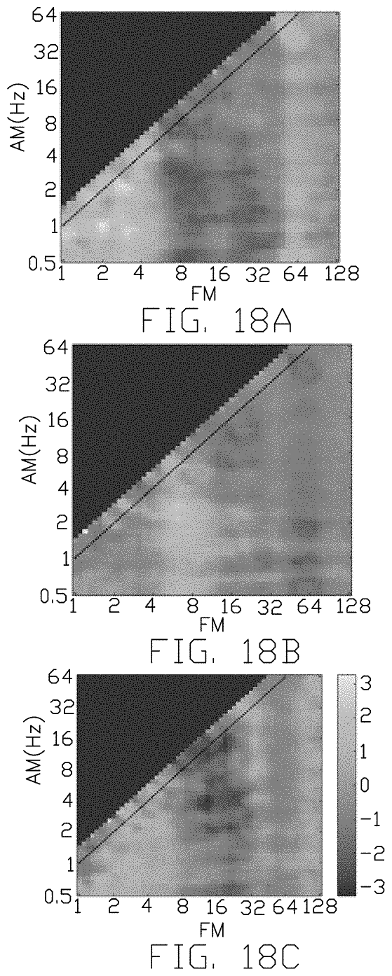

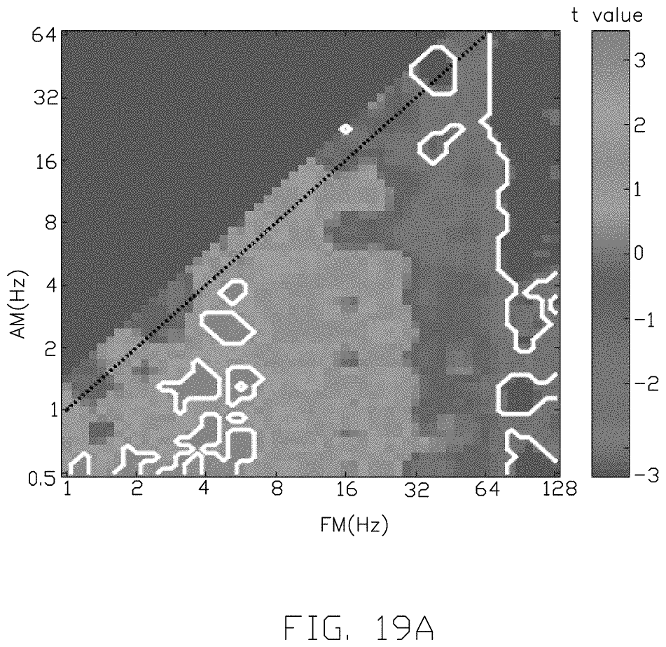

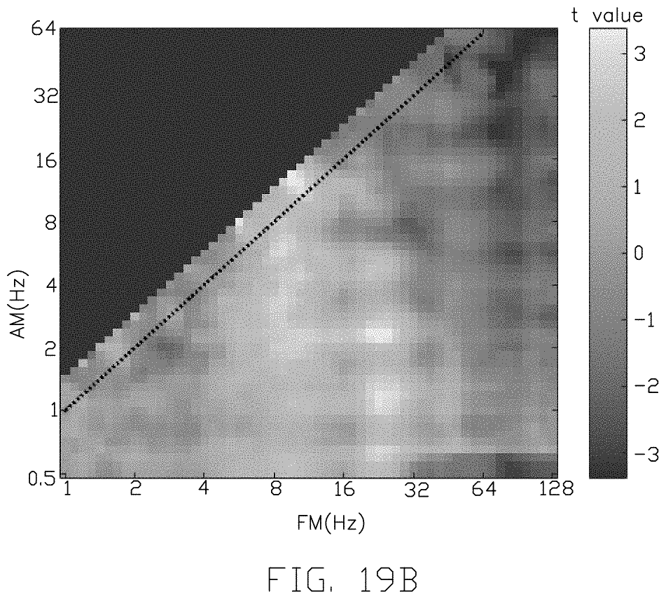

FIG. 18A, FIG. 18B, and FIG. 18C are marked-up visual outputs of the fEEGs for detection or diagnosis of dementia, in accordance with embodiments of the present disclosure.

FIG. 19A and FIG. 19B are marked-up visual outputs of the fEEGs for comparing different subjects with different clinical dementia rating (CDR); in accordance with embodiments of the present disclosure.

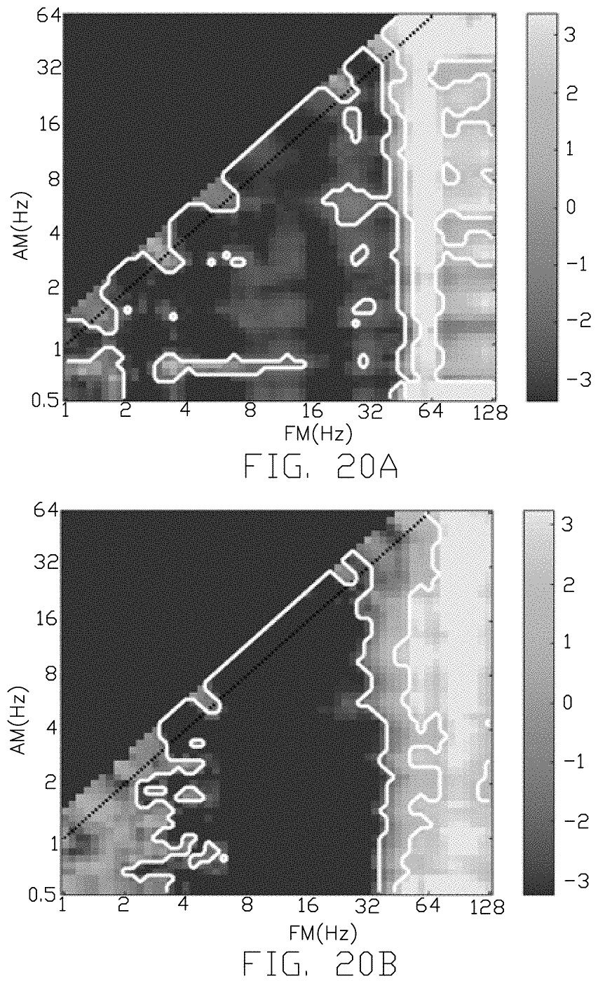

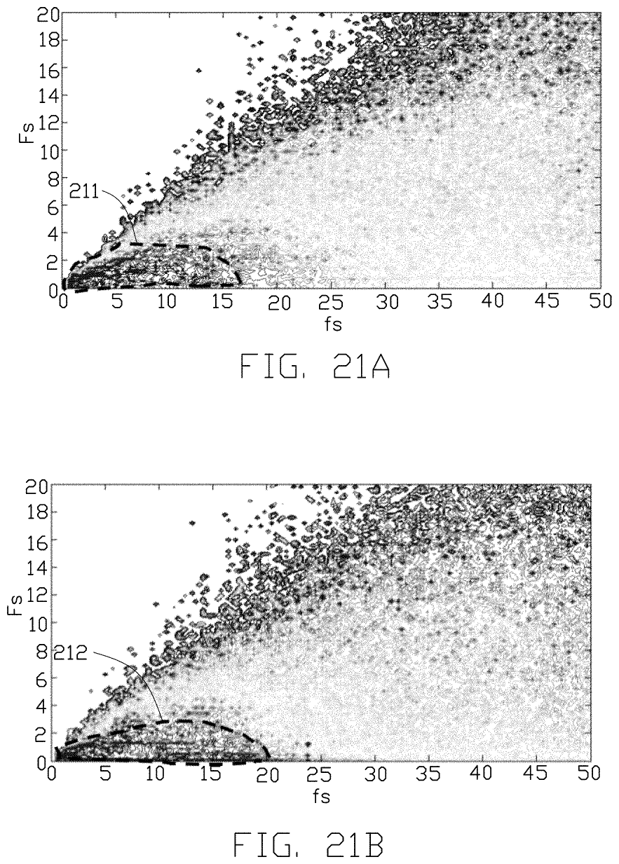

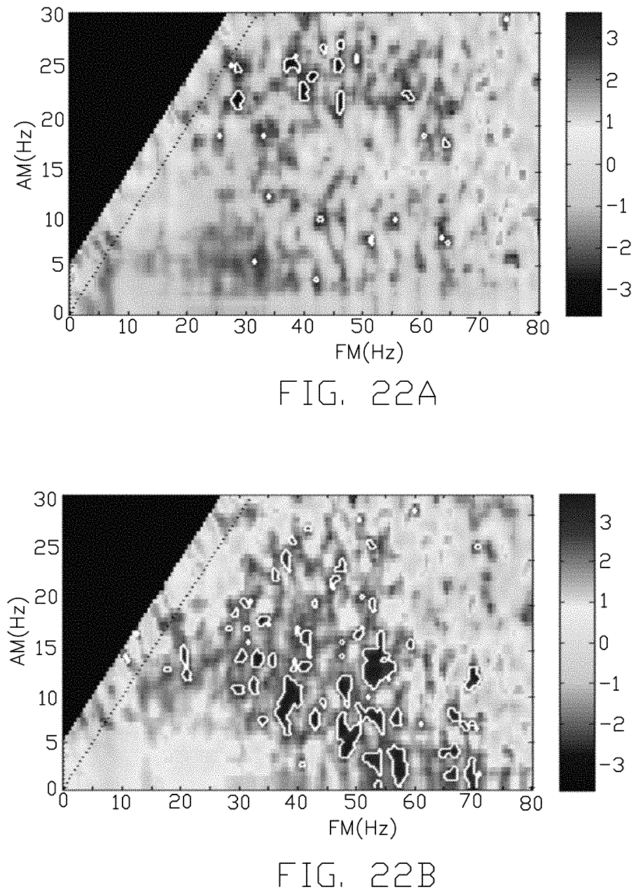

FIG. 20A and FIG. 20B are marked-up visual outputs of the fEEGs for detection or diagnosis of Huntington's Disease (HD), in accordance with embodiments of the present disclosure.

FIG. 21A and FIG. 21B are marked-up visual outputs of the fEEGs for detection or diagnosis of depression or mood disorder, in accordance with embodiments of the present disclosure.

FIG. 22A and FIG. 22B are marked-up visual outputs of the fEEGs for detection or diagnosis of migraine headache, in accordance with embodiments of the present disclosure.

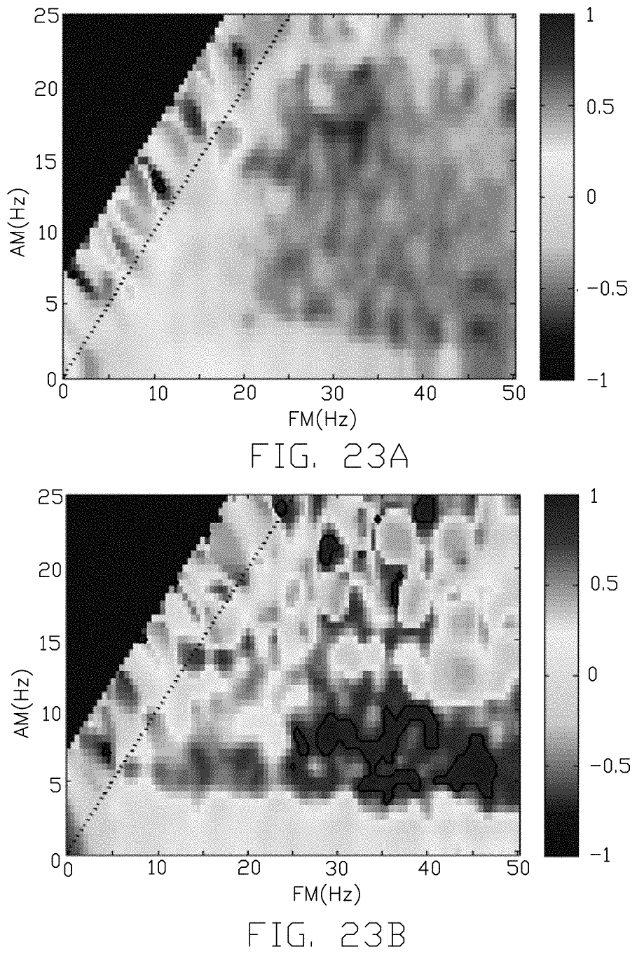

FIG. 23A and FIG. 23B are marked-up visual outputs of the fEEGs for correlating headache frequency and the fEEG, in accordance with embodiments of the present disclosure.

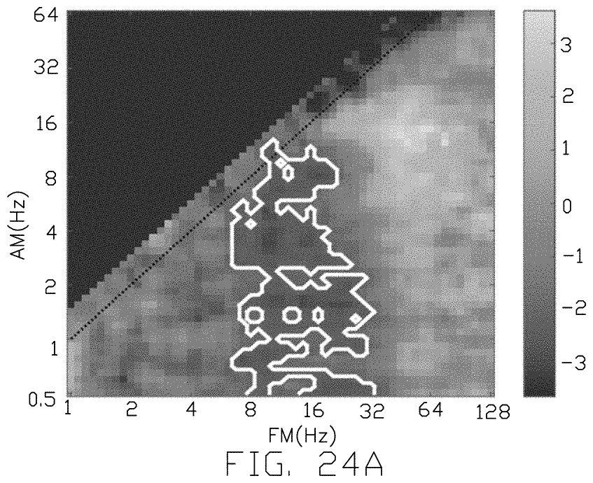

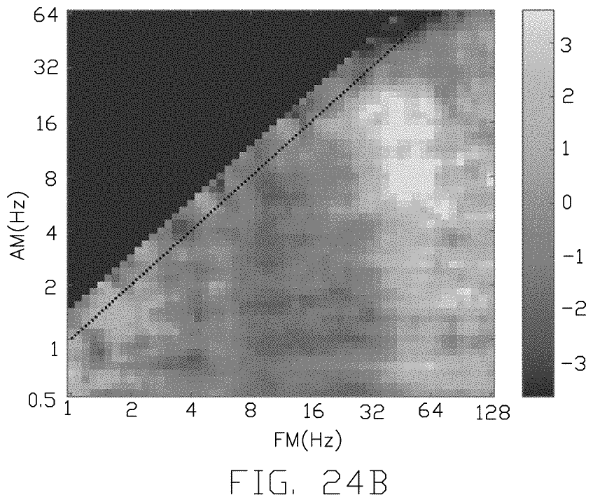

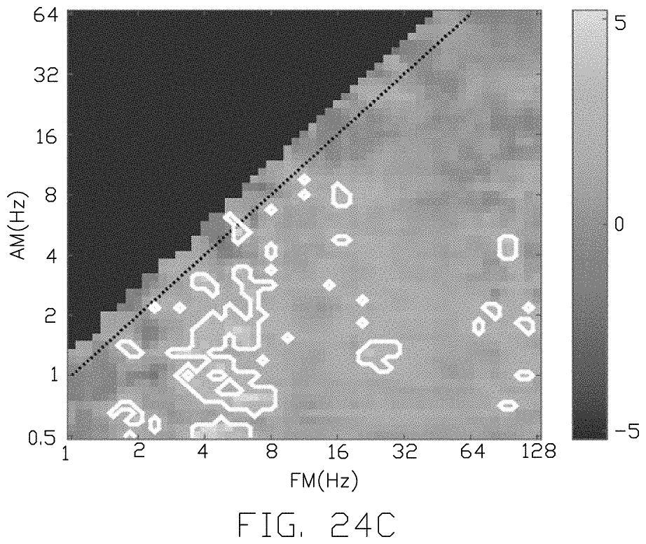

FIG. 24A, FIG. 24B, and FIG. 24C are marked-up visual outputs of the fEEGs on treatment response for migraine headache, in accordance with embodiments of the present disclosure.

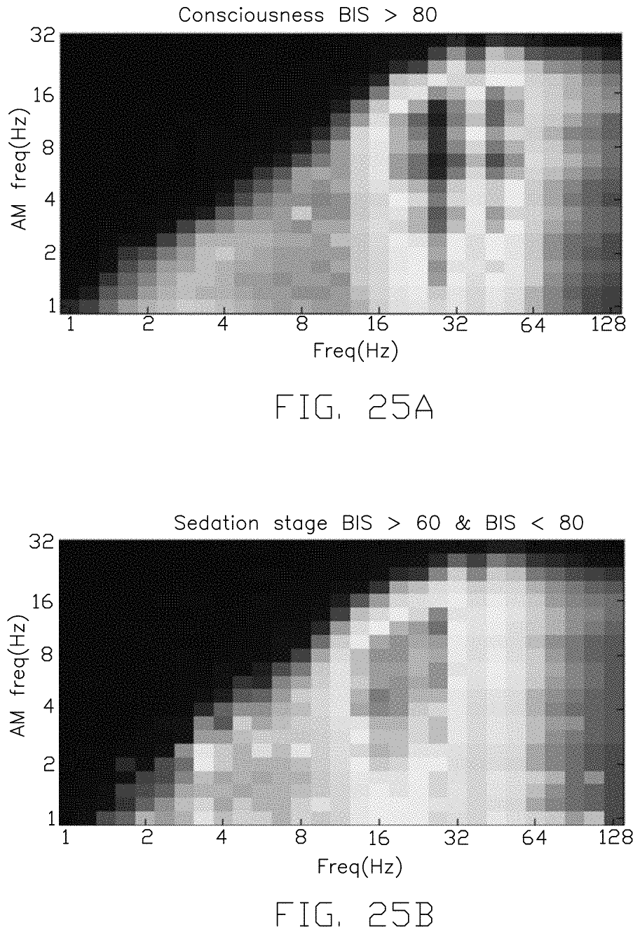

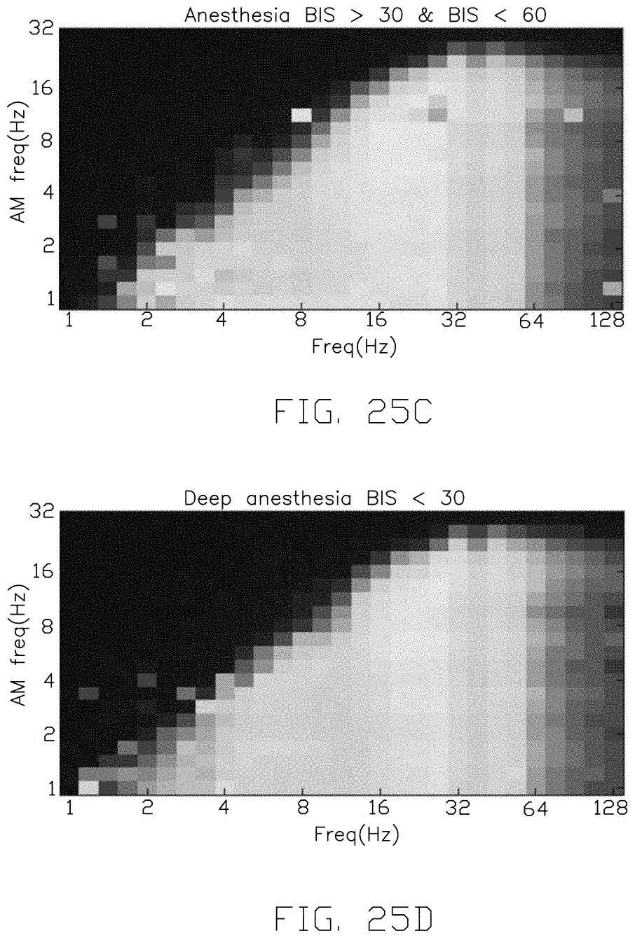

FIG. 25A, FIG. 25B, FIG. 25C, and FIG. 25D are visual outputs of the fEEGs on the evaluation or monitoring of anesthesia depth, in accordance with embodiments of the present disclosure.

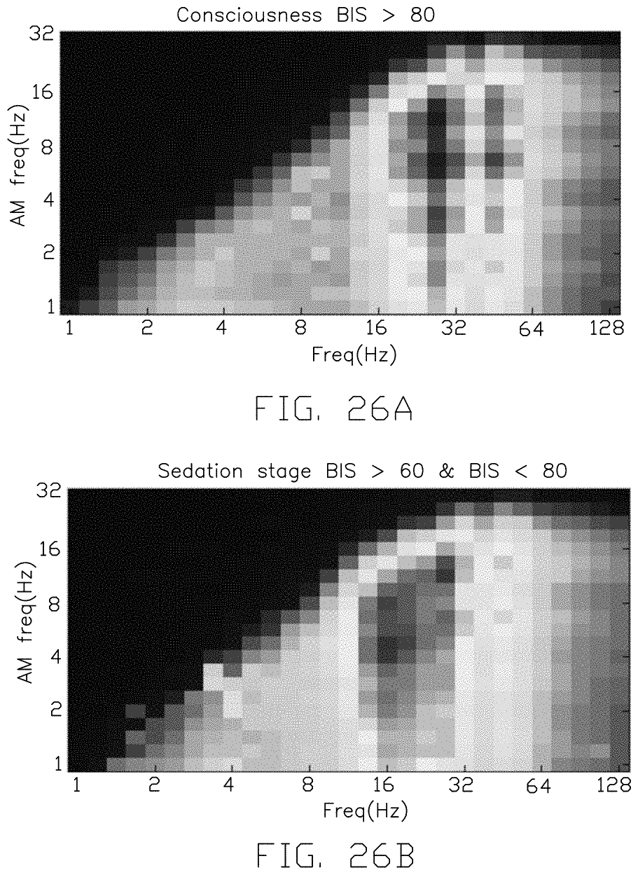

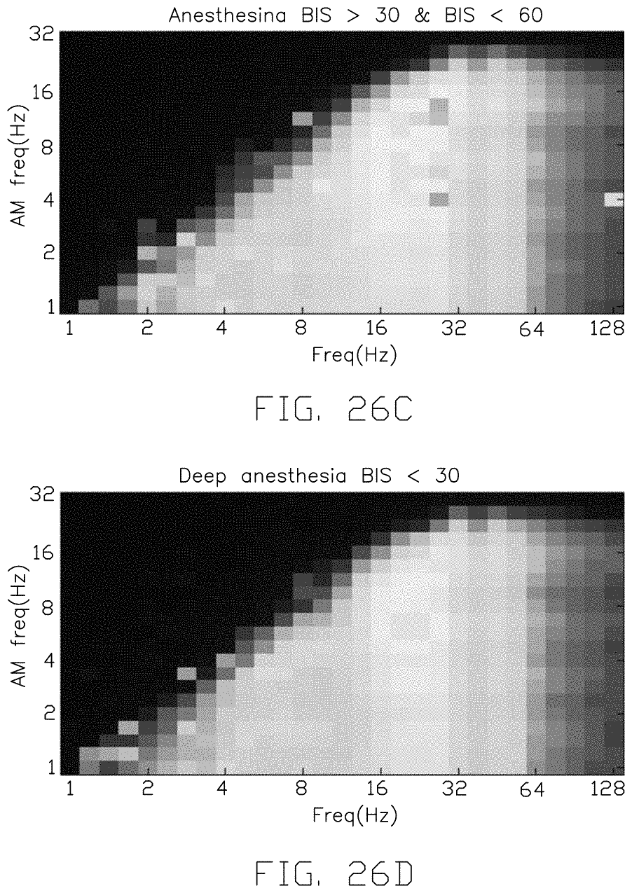

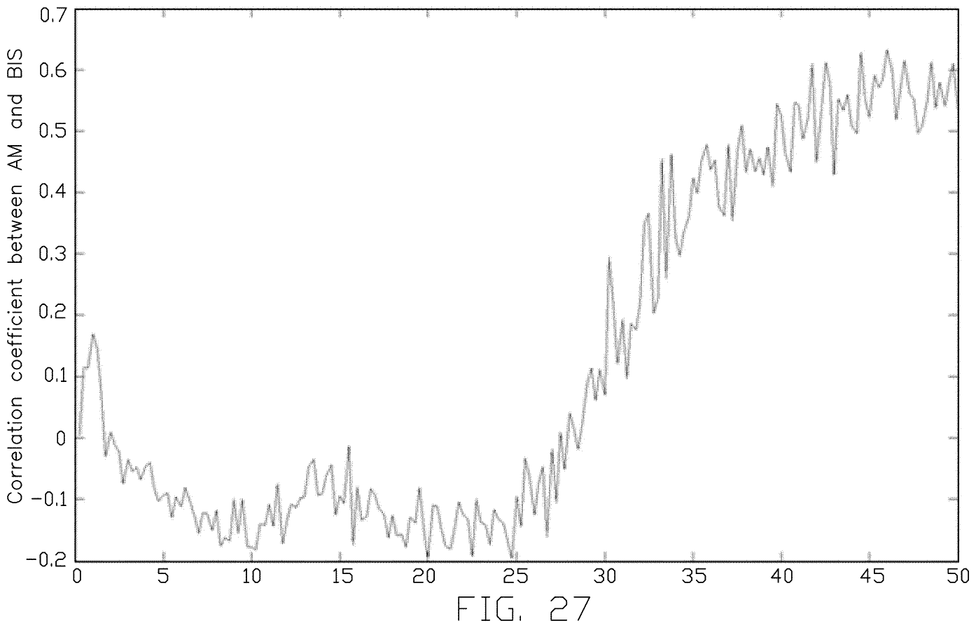

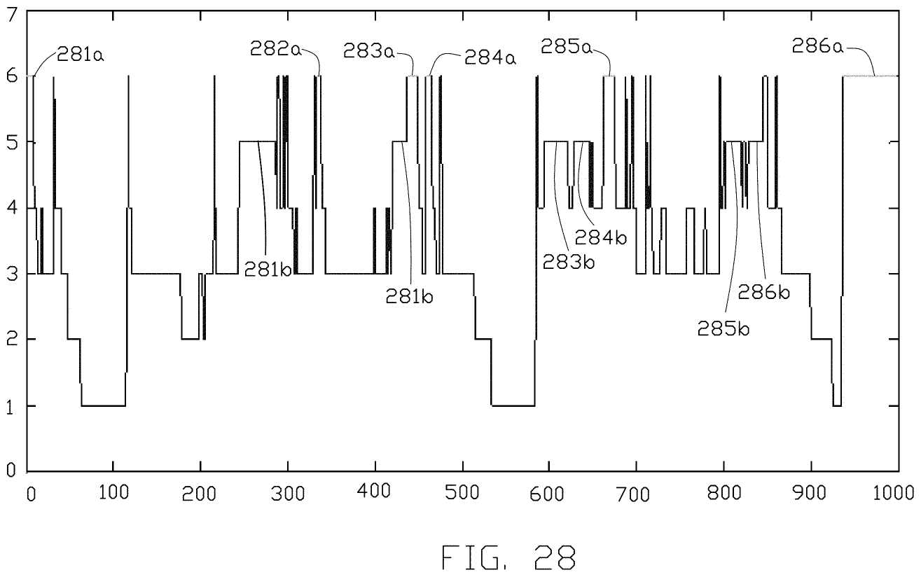

FIG. 26A, FIG. 26B, FIG. 26C, and FIG. 26D are visual outputs of the fEEGs on the evaluation or monitoring of anesthesia depth, in accordance with embodiments of the present disclosure.

FIG. 27 is a correlation graph between Bispectral Index (BIS) and AM on different FM frequencies in the fEEGs, in accordance with embodiments of the present disclosure.

FIG. 28 is a hynograph of a subject in accordance with embodiment of the present disclosure.

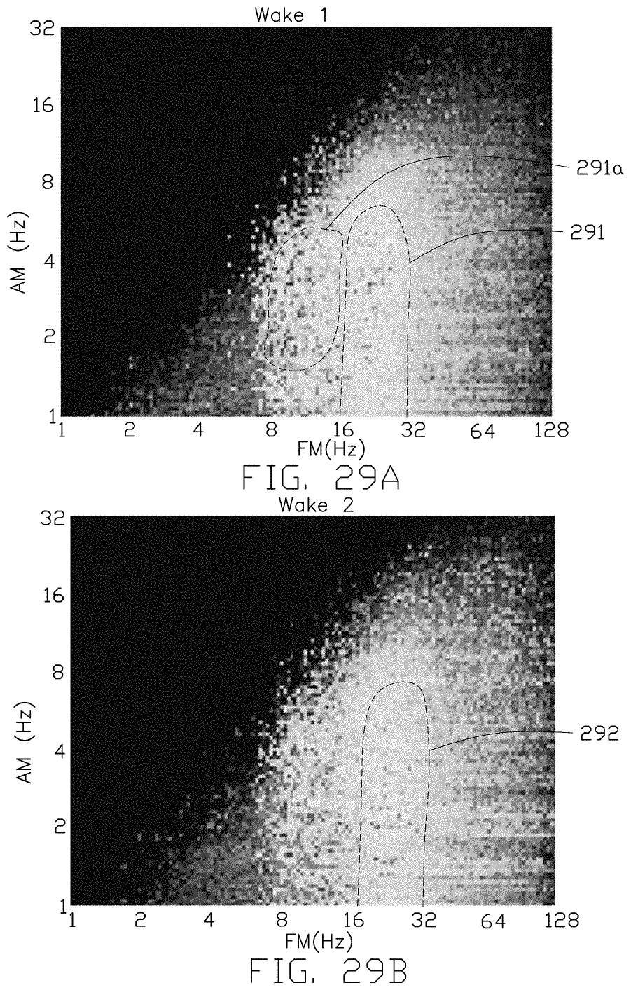

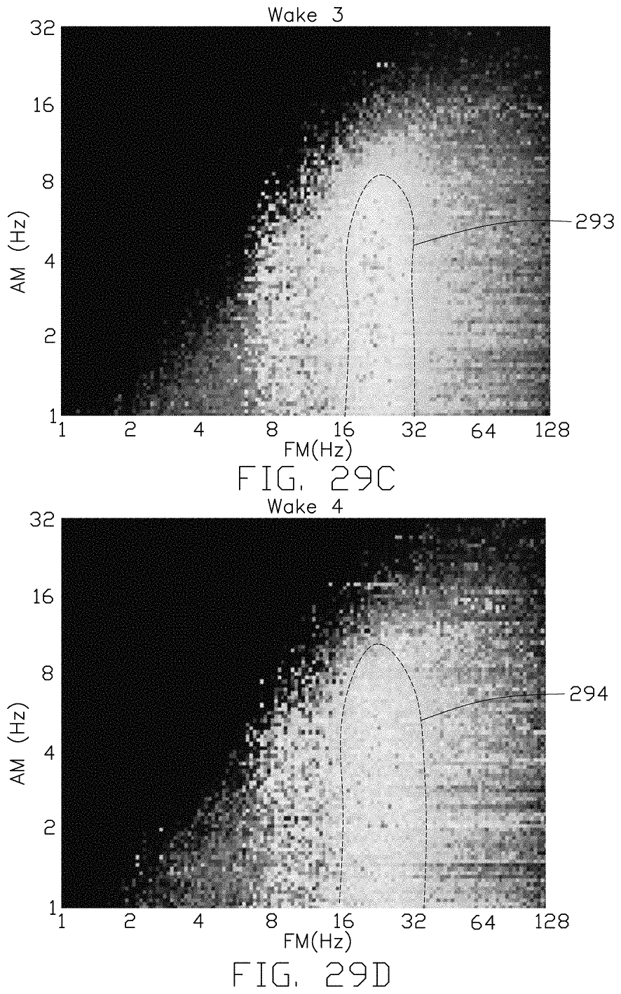

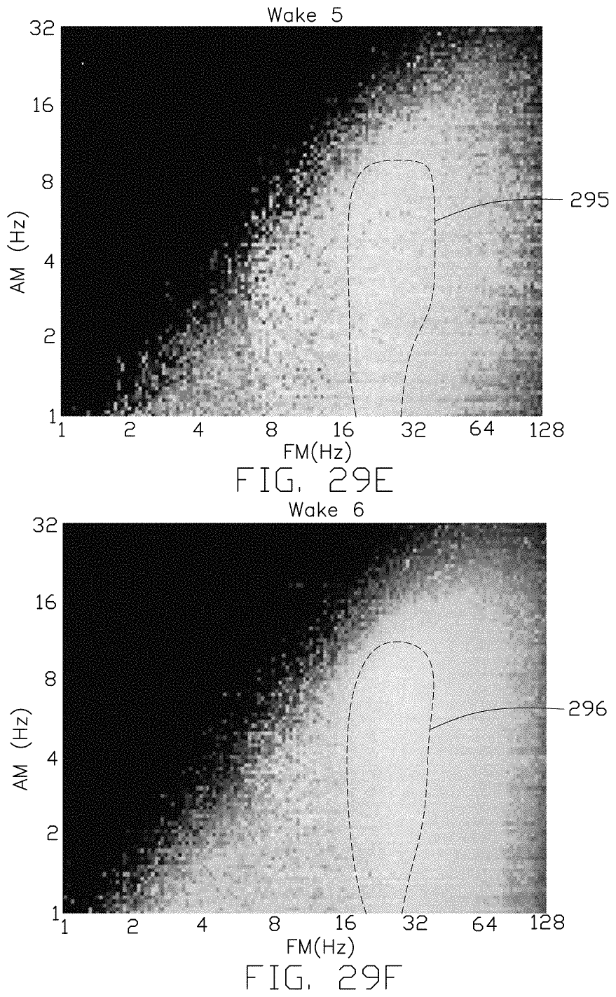

FIG. 29A, FIG. 29B, FIG. 29C, FIG. 29D, FIG. 29E, and FIG. 29F are marked-up visual outputs of the fEEGs corresponding to different awake stages, in accordance with embodiments of the present disclosure.

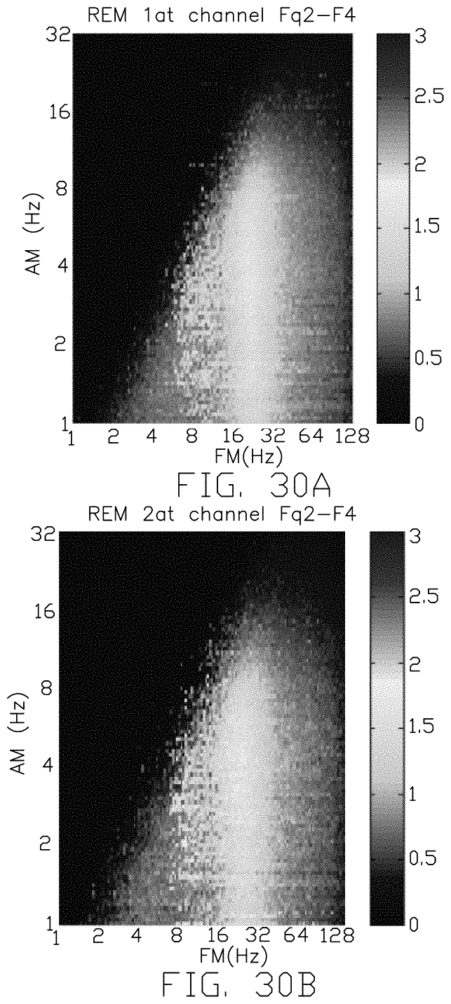

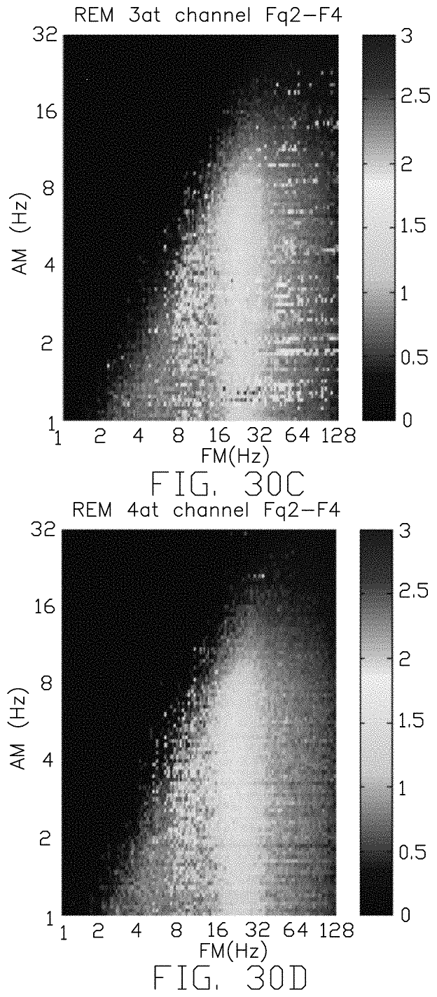

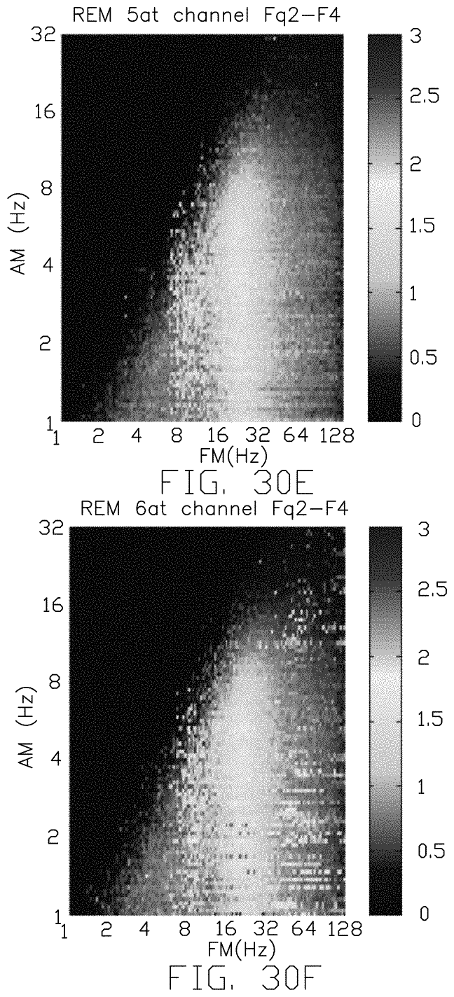

FIG. 30A, FIG. 30B, FIG. 30C, FIG. 30D, FIG. 30E, and FIG. 30F are visual outputs of the fEEGs corresponding to different REM stages, in accordance with embodiments of the present disclosure.

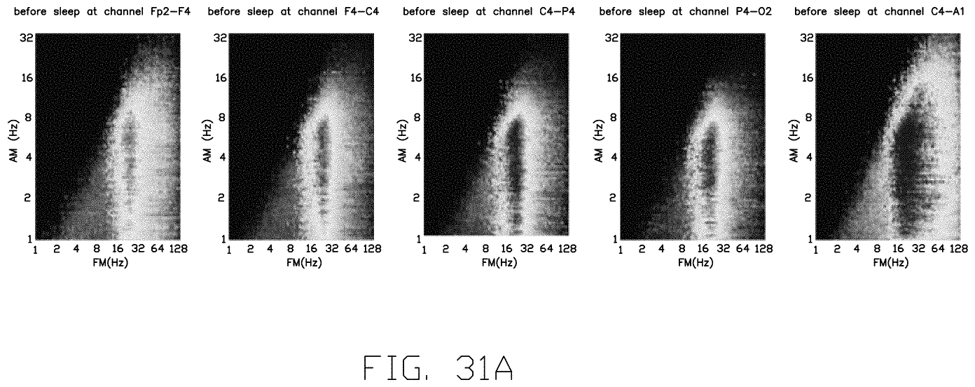

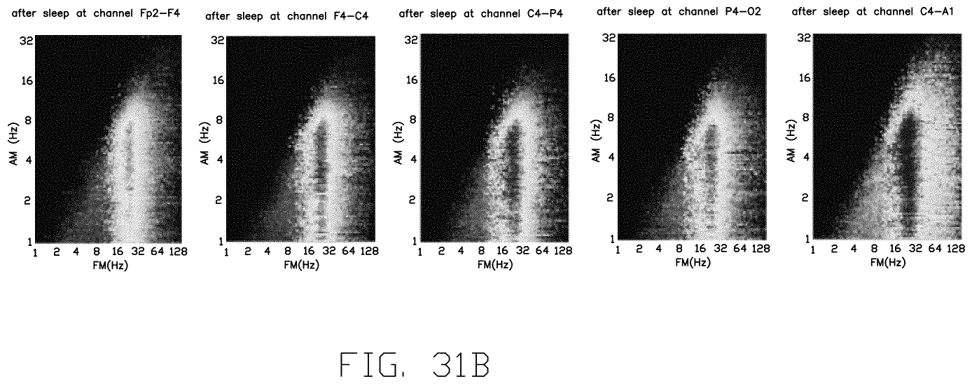

FIG. 31A and FIG. 31B are visual outputs of the fEEGs on evaluation or monitoring insomnia, in accordance with embodiments of the present disclosure.

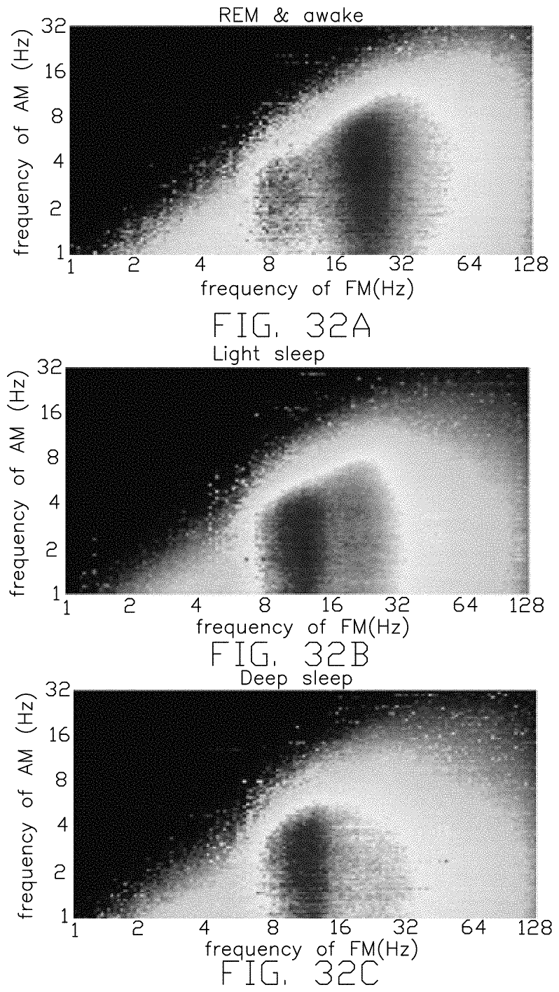

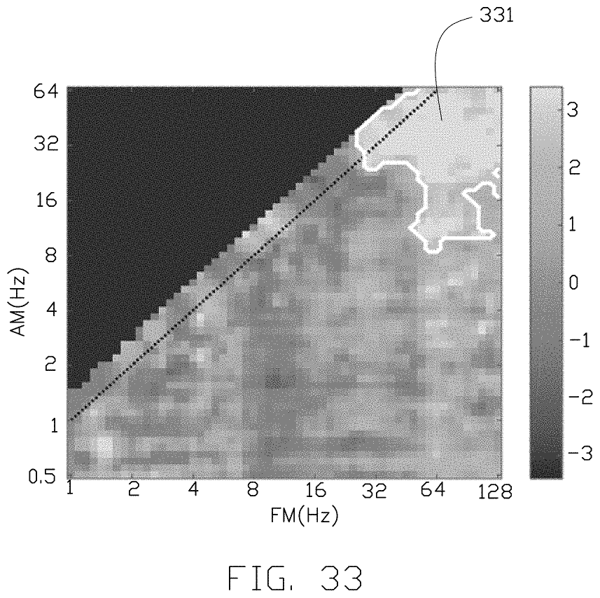

FIG. 32A, FIG. 32B, and FIG. 32C are visual outputs of the fEEGs on evaluation or monitoring insomnia, in accordance with embodiments of the present disclosure.

FIG. 33 is a marked-up visual output of the fEEG on detection or diagnosis of Parkinson's Disease (PD) in accordance with an embodiment of the present disclosure.

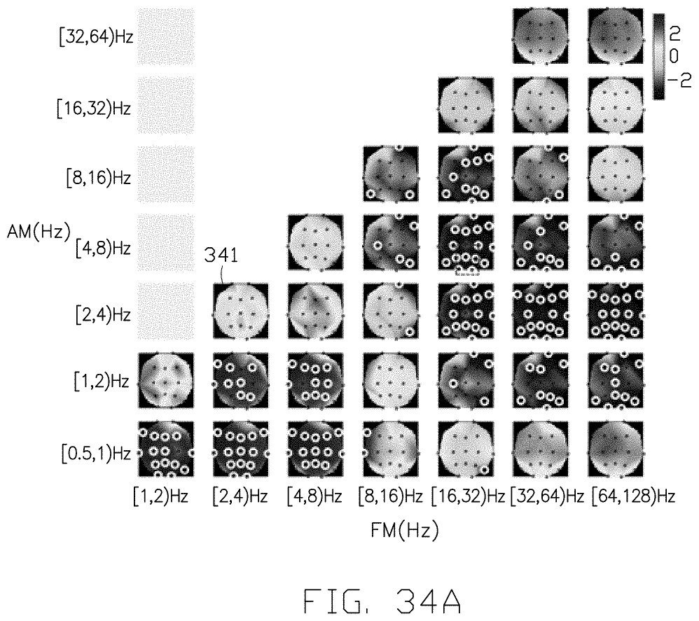

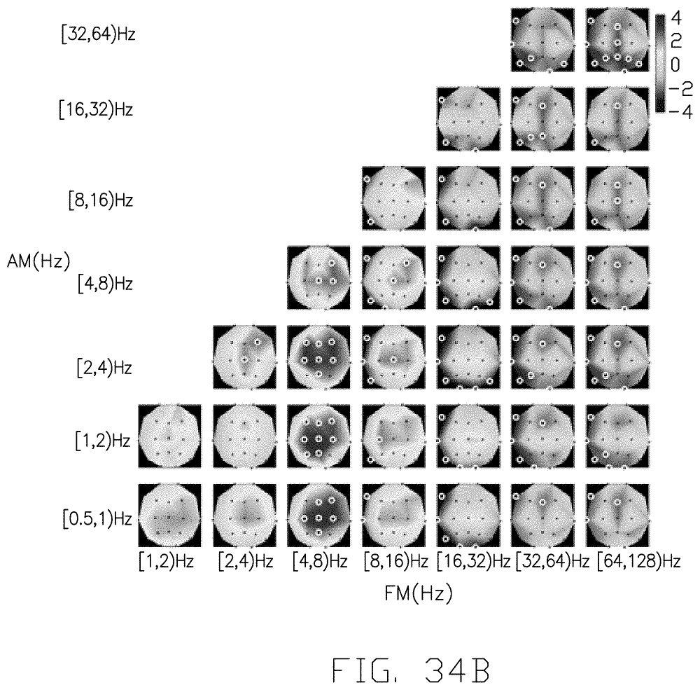

FIG. 34A and FIG. 34B are visual outputs of the fEEToPGs comprising a plurality of topographies of the brain, in accordance with embodiments of the present disclosure.

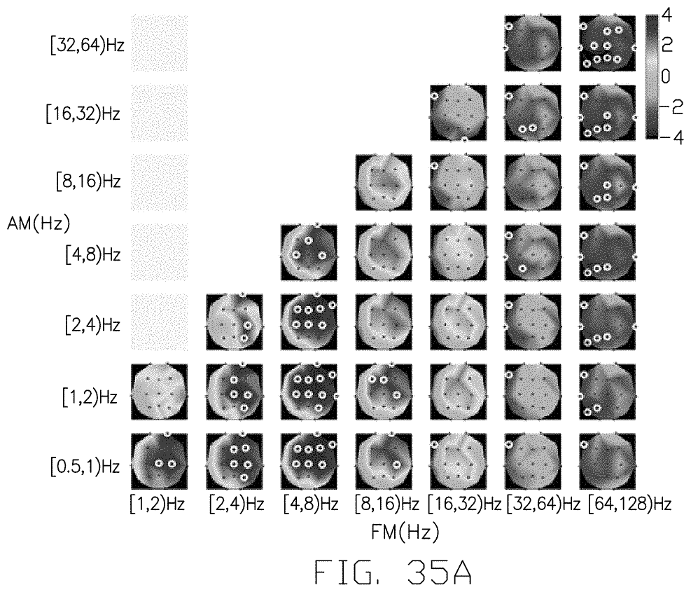

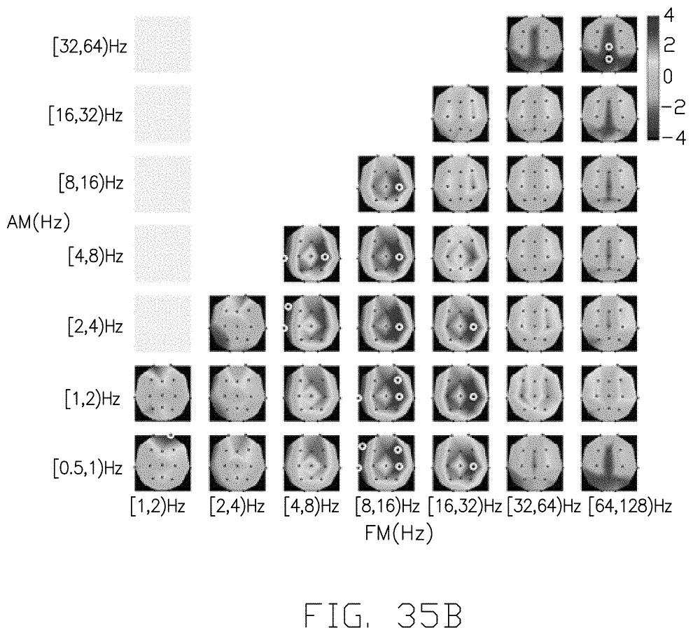

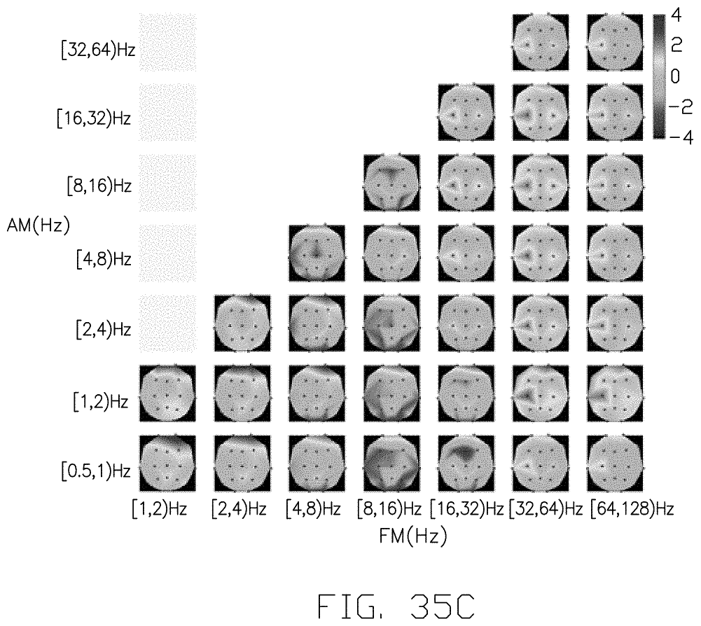

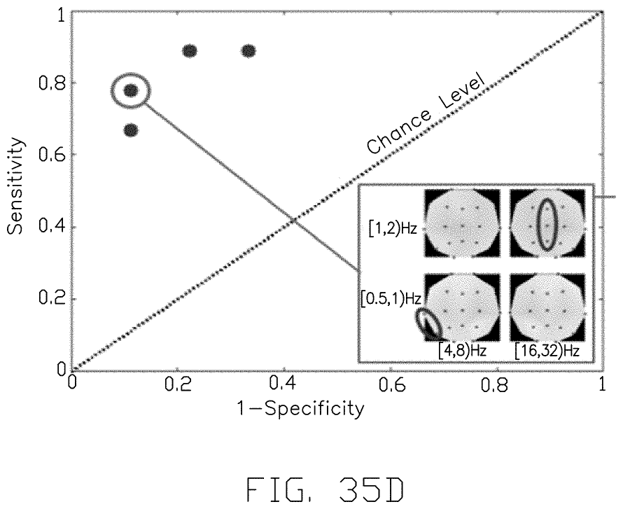

FIG. 35A, FIG. 35B, and FIG. 35C are visual outputs of the fEEToPGs comprising a plurality of topographies of the brain, in accordance with embodiments of the present disclosure. FIG. 35D is a marked-up receiver operating curve (ROC) illustration for the utility of the fEEToPG, in accordance with an embodiment of the present disclosure.

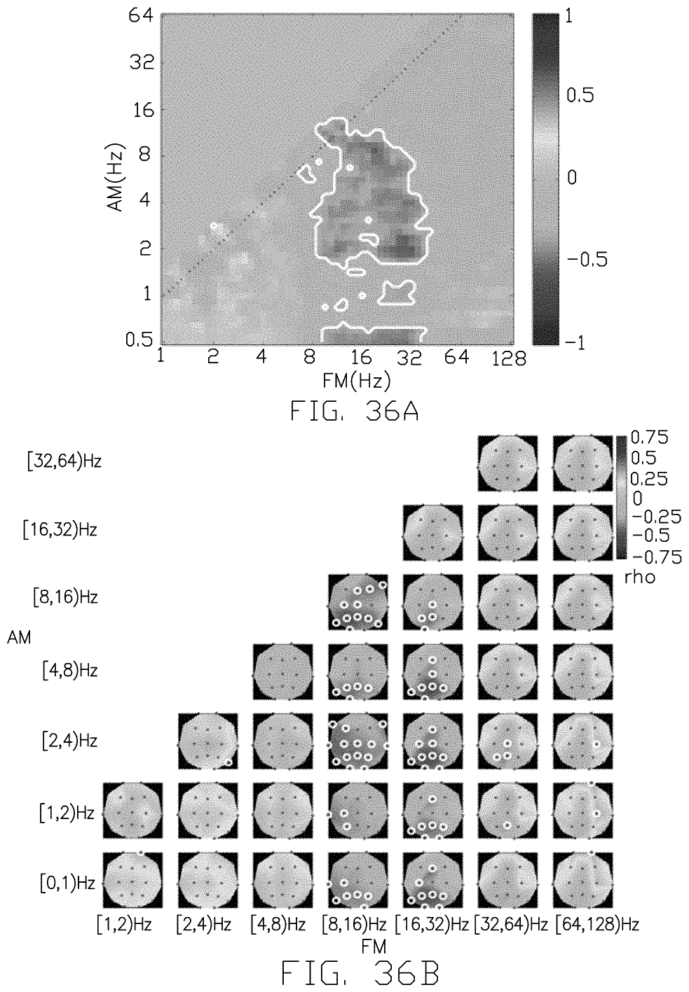

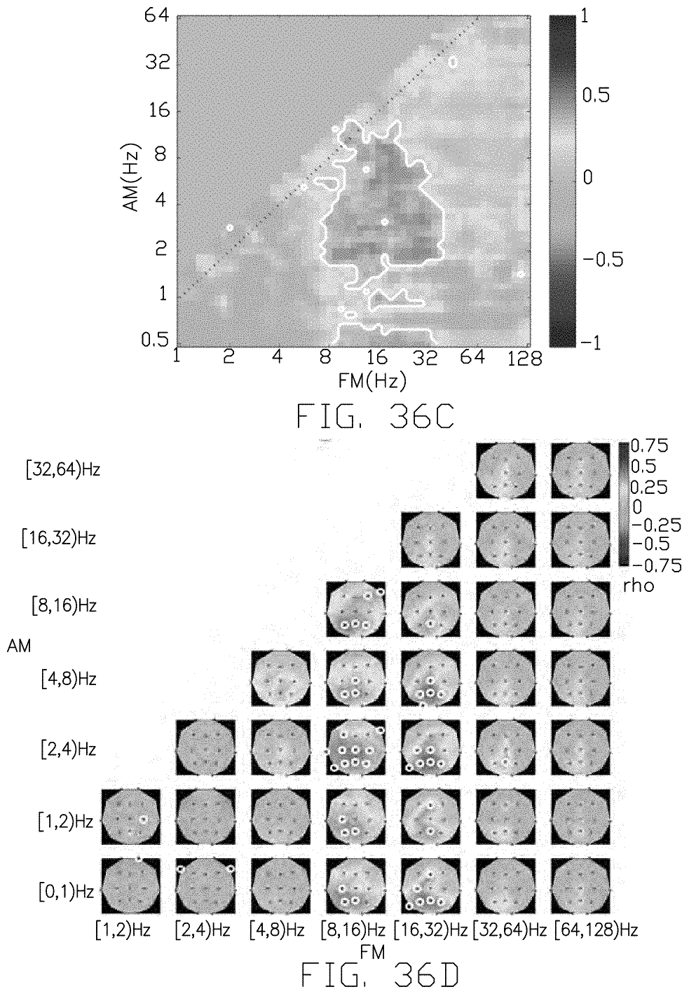

FIG. 36A and FIG. 36C are marked-up visual outputs of the fEEGs, and FIG. 36B and FIG. 36D are visual outputs of the fEEToPGs comprising a plurality of topographies of the brain on evaluation the progression of the diseases status of Alzheimer's Disease (AD), in accordance with embodiments of the present disclosure.

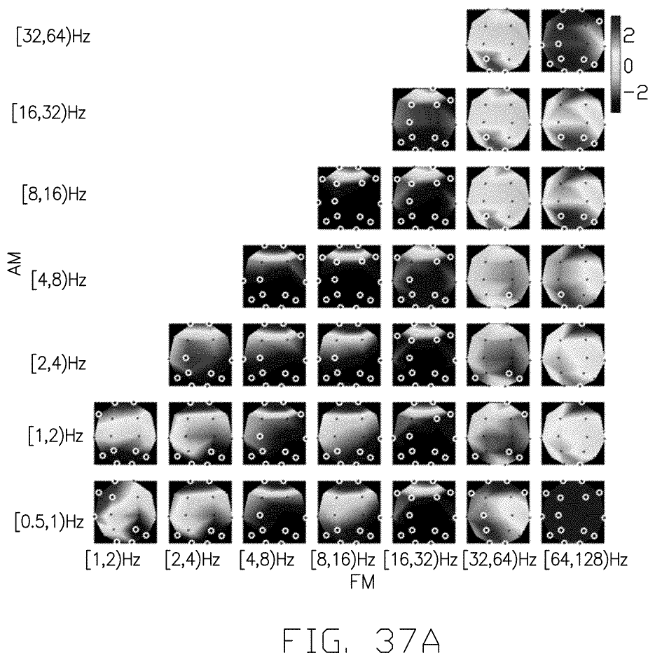

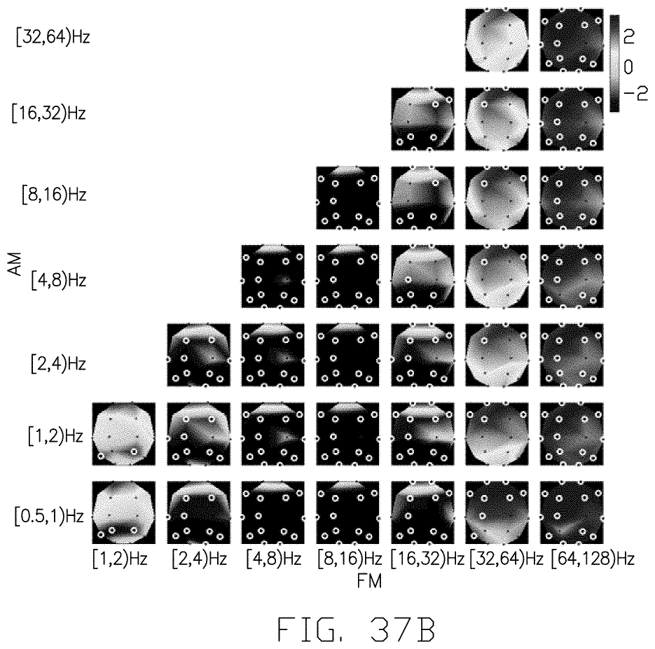

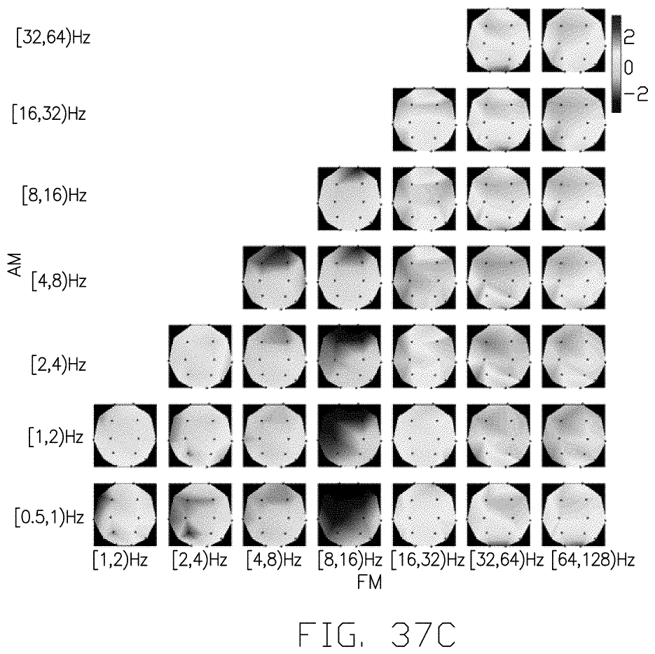

FIG. 37A, FIG. 37B, and FIG. 37C are visual outputs of the fEEToPGs comprising a plurality of topographies of the brain on detection or diagnosis of HD, in accordance with embodiments of the present disclosure.

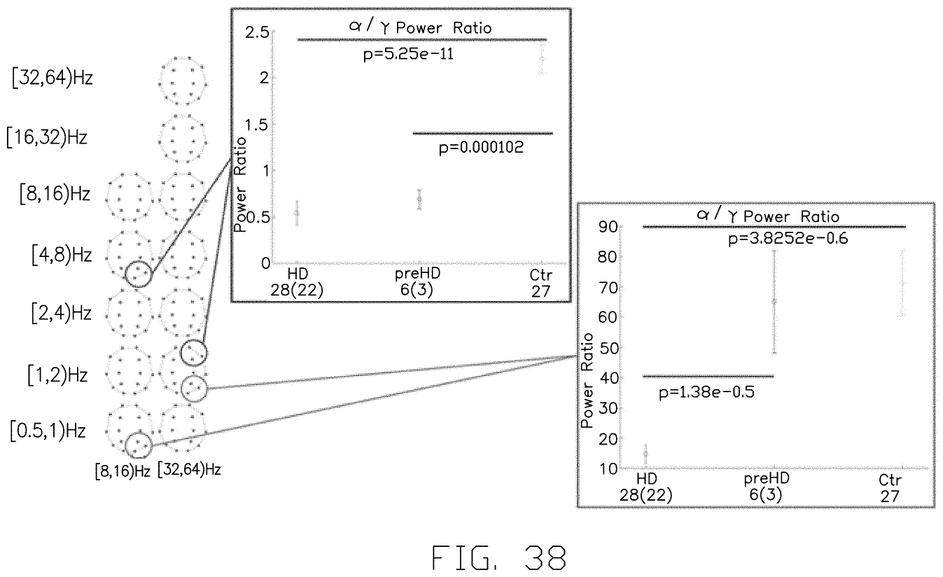

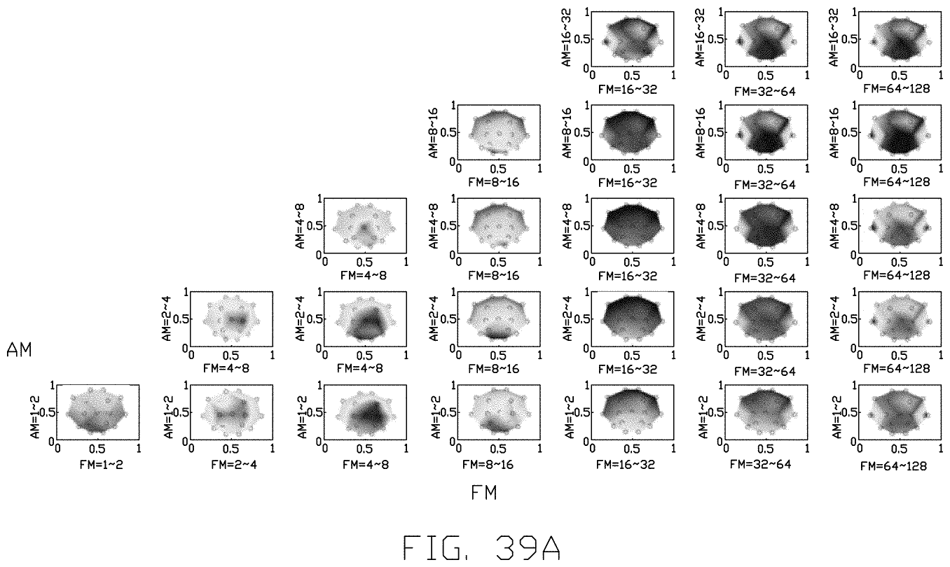

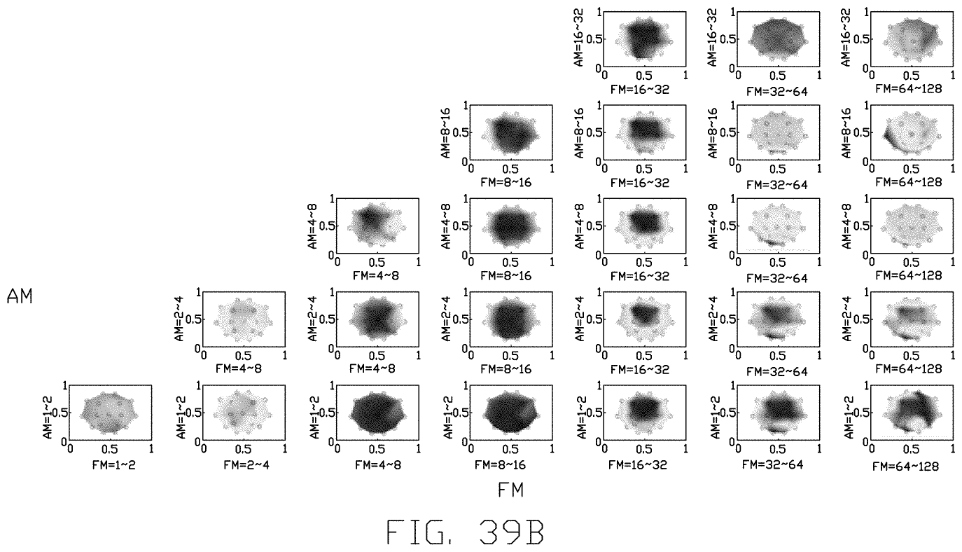

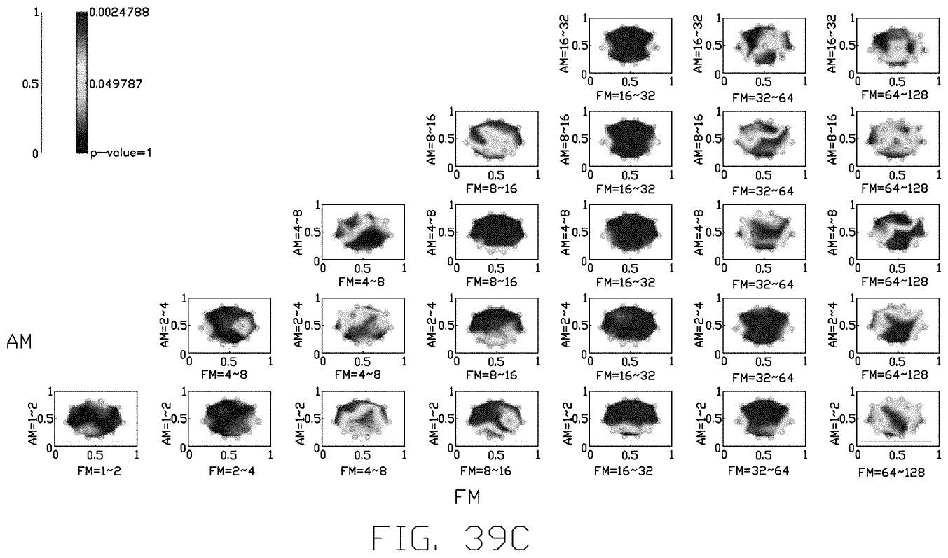

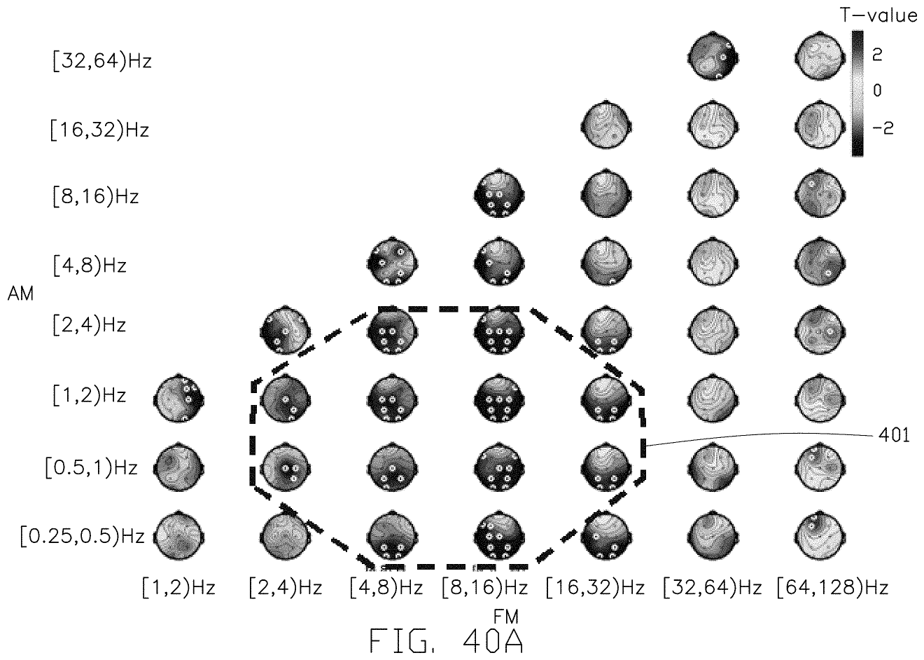

FIG. 38 is a marked-up power ratio index graph on detection or diagnosis of HD in accordance with embodiments of the present disclosure.

FIG. 39A, FIG. 39B, and FIG. 39C are visual outputs of the fEEToPGs comprising a plurality of topographies of the brain on detection or diagnosis of depression or mood disorder, in accordance with embodiments of the present disclosure.

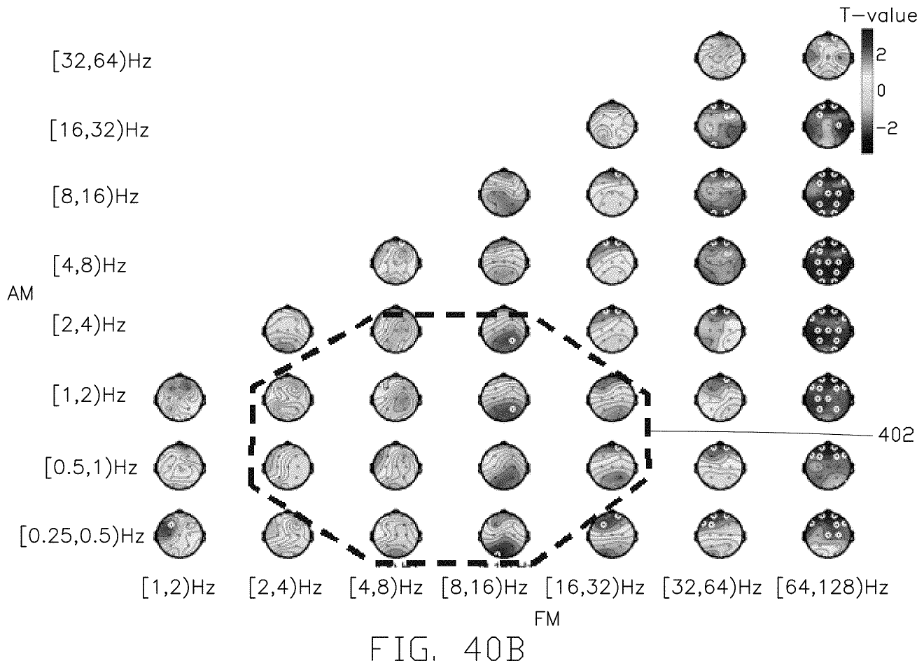

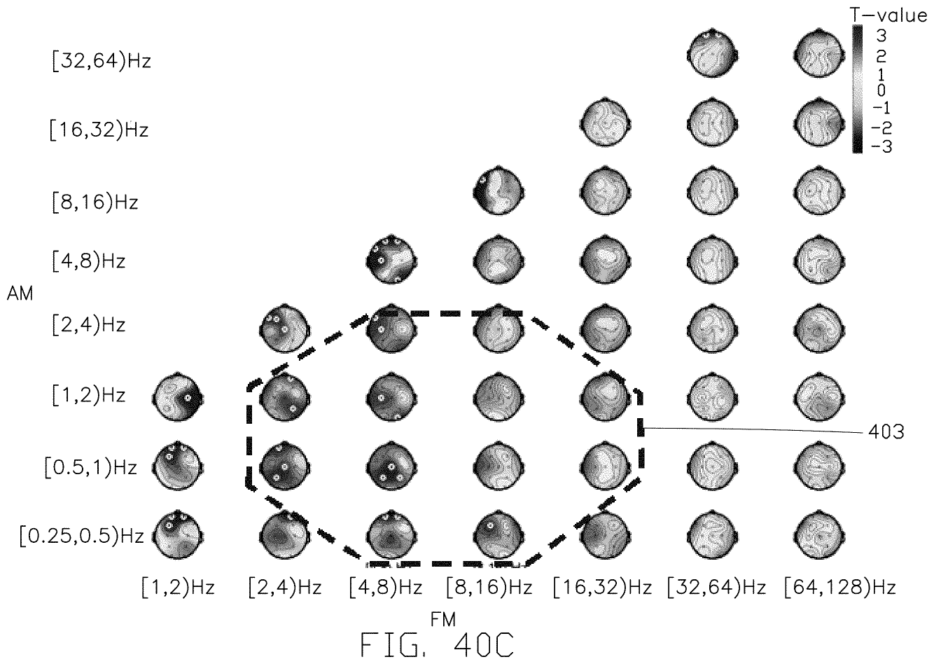

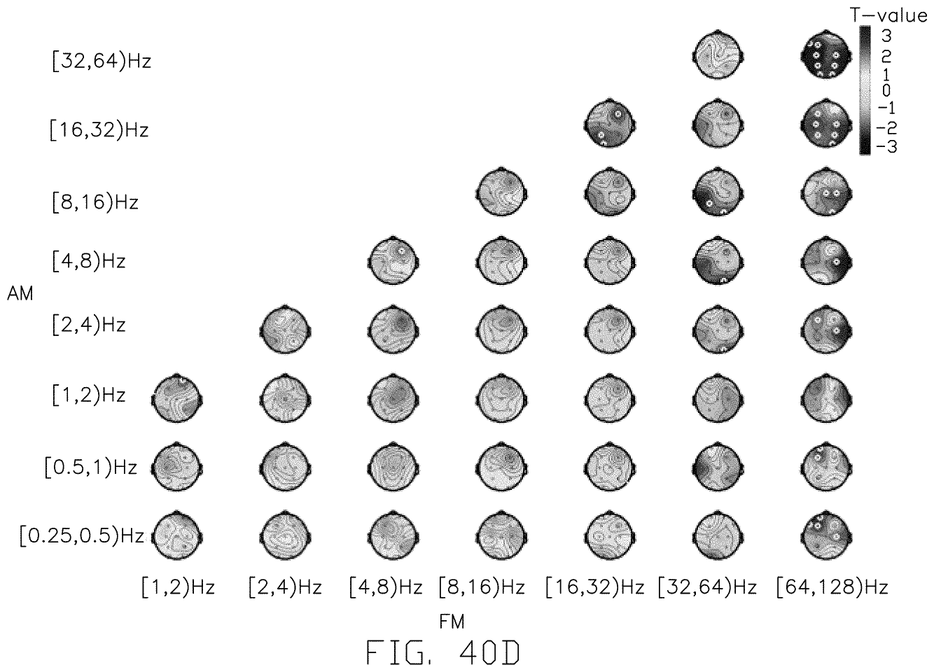

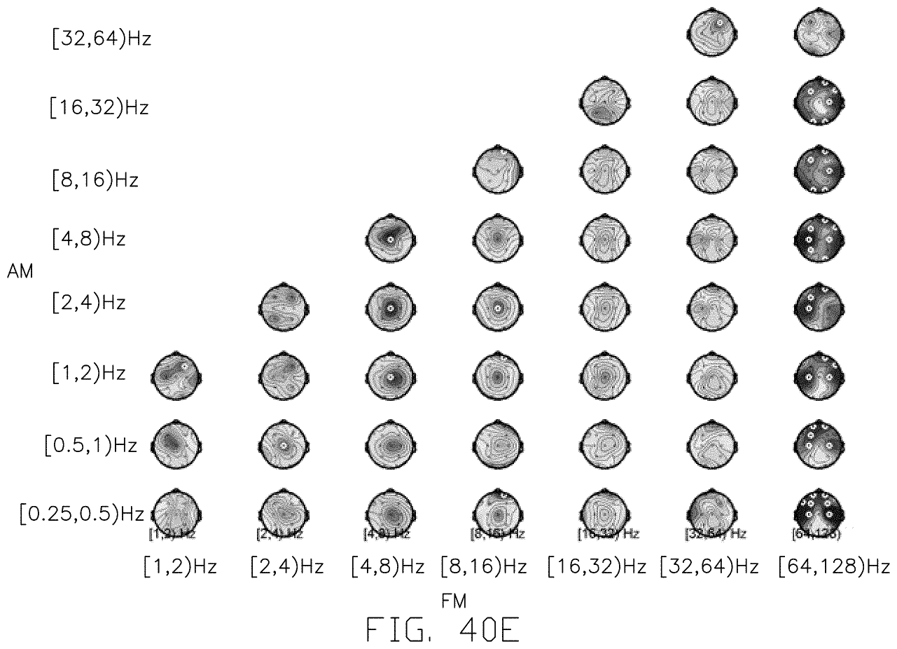

FIG. 40A, FIG. 40B, and FIG. 40C are marked-up visual outputs of the fEEToPGs comprising a plurality of topographies on detection or diagnosis of migraine and treatment response thereof, and FIG. 40D, and FIG. 40E are visual outputs of the fEEToPGs comprising a plurality of topographies on detection or diagnosis of migraine and treatment responses thereof, in accordance with embodiments of the present disclosure.

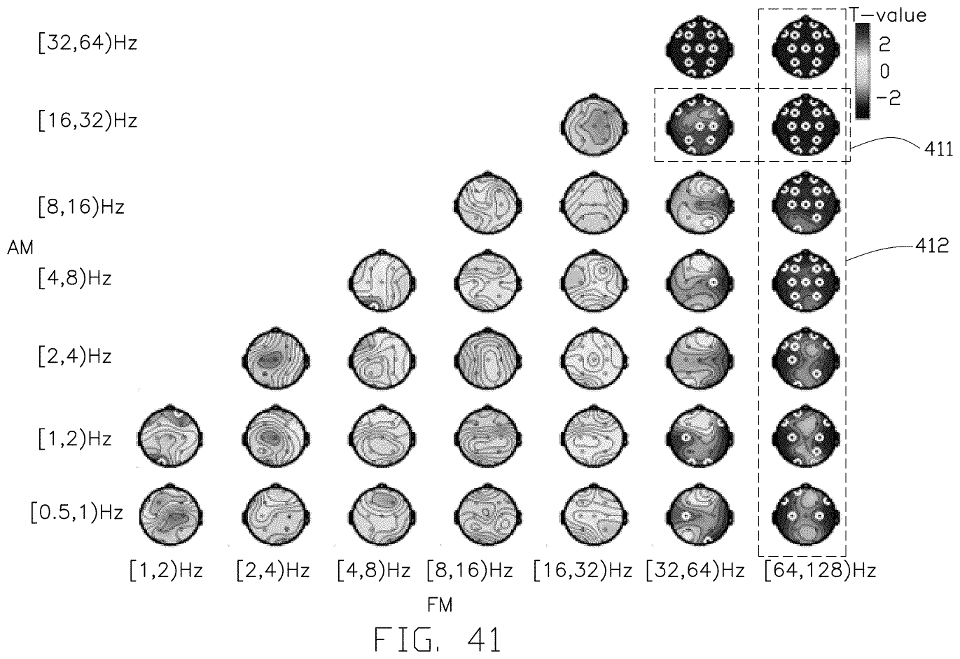

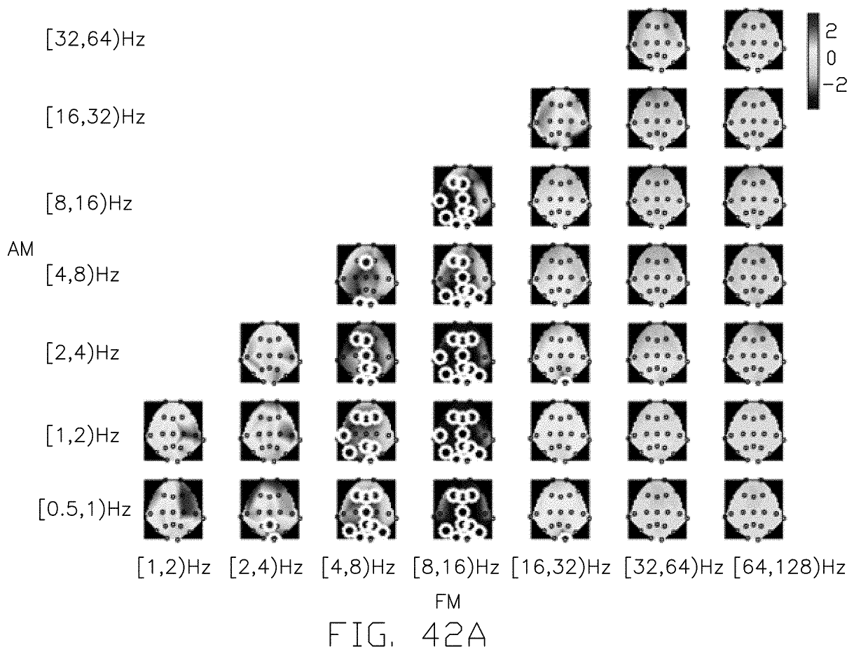

FIG. 41 is a marked-up visual output of the fEEToPGs comprising a plurality of topographies of the brain on detection or diagnosis of Parkinson's Disease (PD) in accordance with an embodiment of the present disclosure.

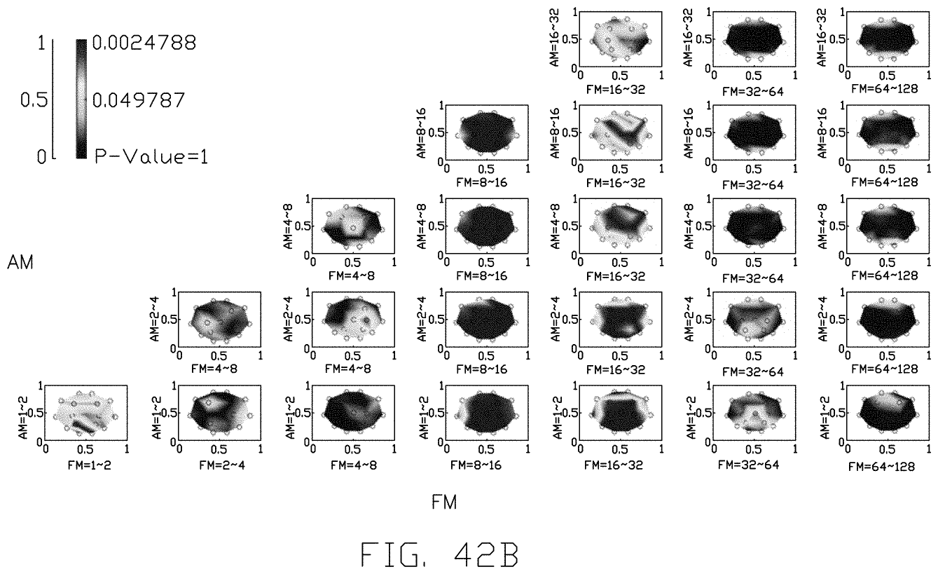

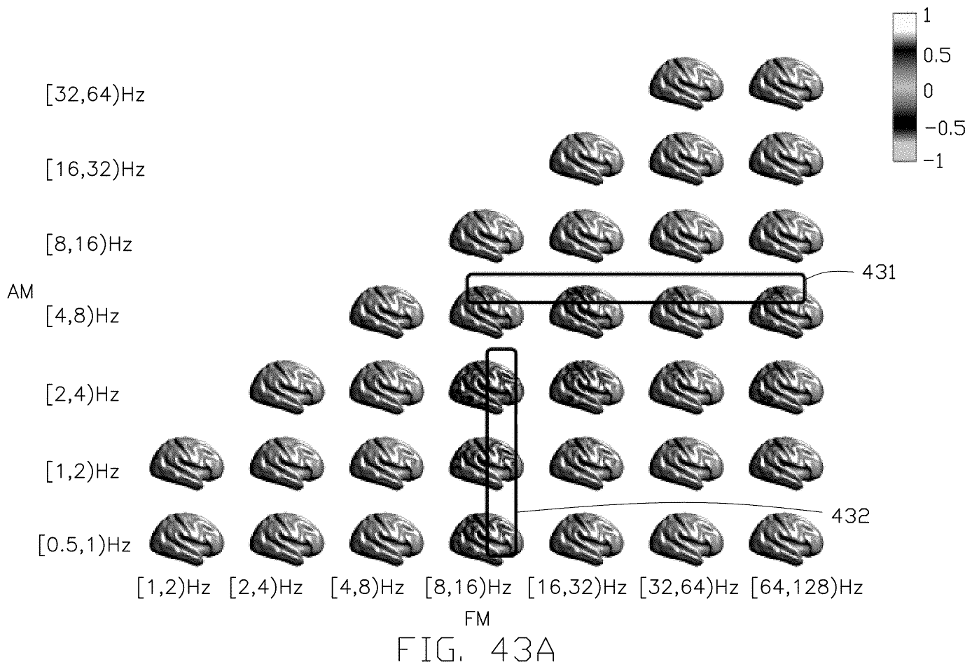

FIG. 42A and FIG. 42B are visual outputs of the fEEToPGs comprising a plurality of topographies of the brain on detection or diagnosis of Attention Deficit Hyperactivity Disorder (ADHD), in accordance with embodiments of the present disclosure.

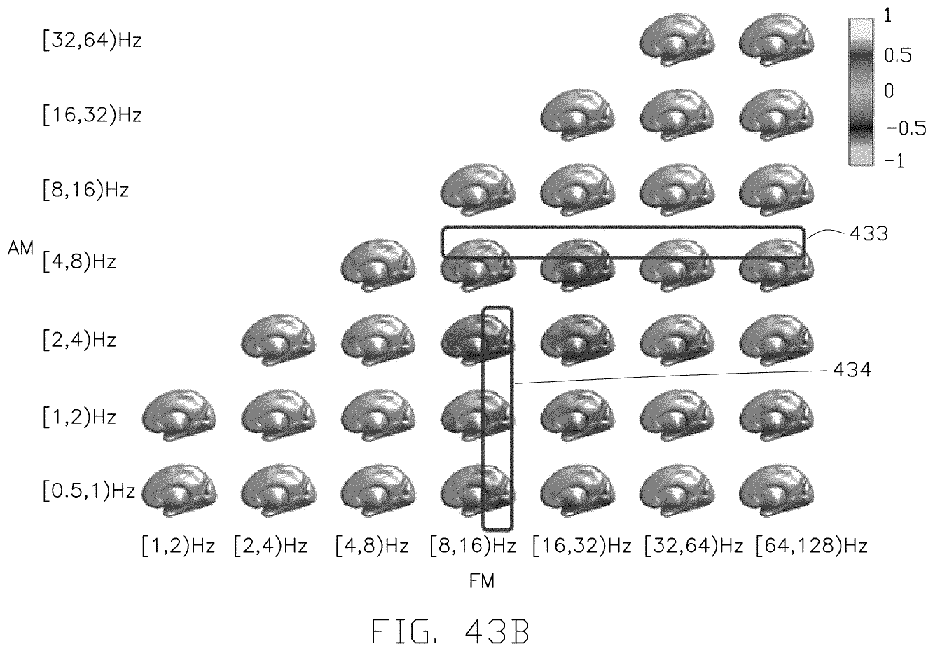

FIG. 43A and FIG. 43B are marked-up visual outputs of the fEEToMGs comprising a plurality of tomographies of the brain for evaluating the progression of AD, in accordance with embodiments of the present disclosure.

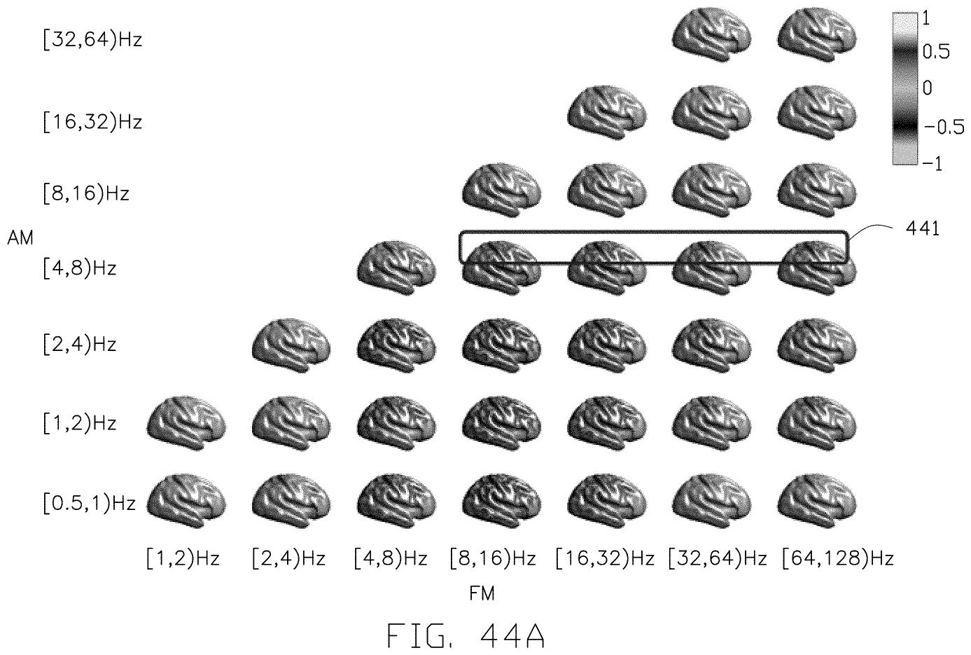

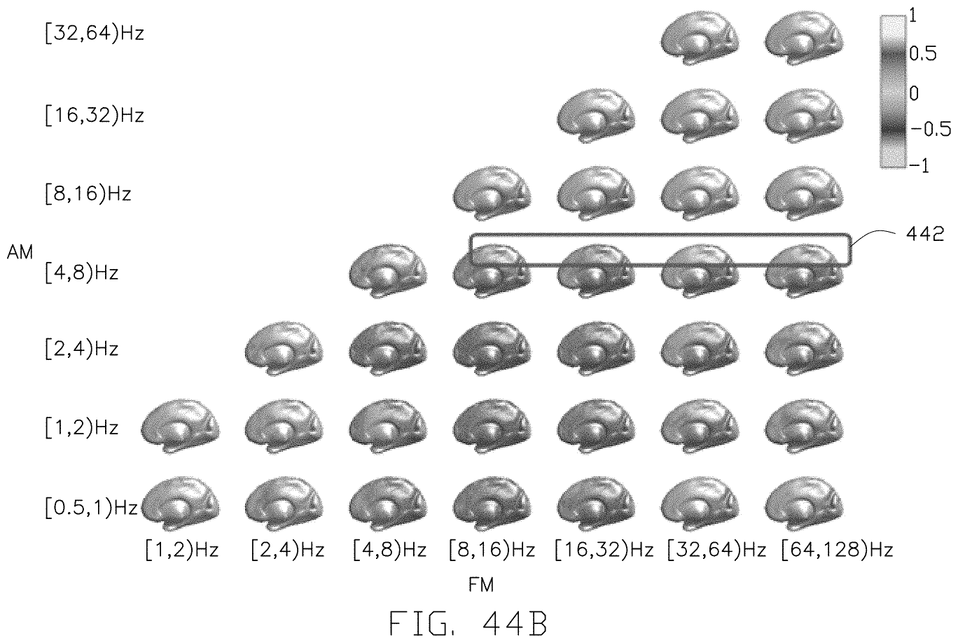

FIG. 44A and FIG. 44B are marked-up visual outputs of the fEEToMGs comprising a plurality of tomographies of the brain for evaluating the progression of AD, in accordance with embodiments of the present disclosure.

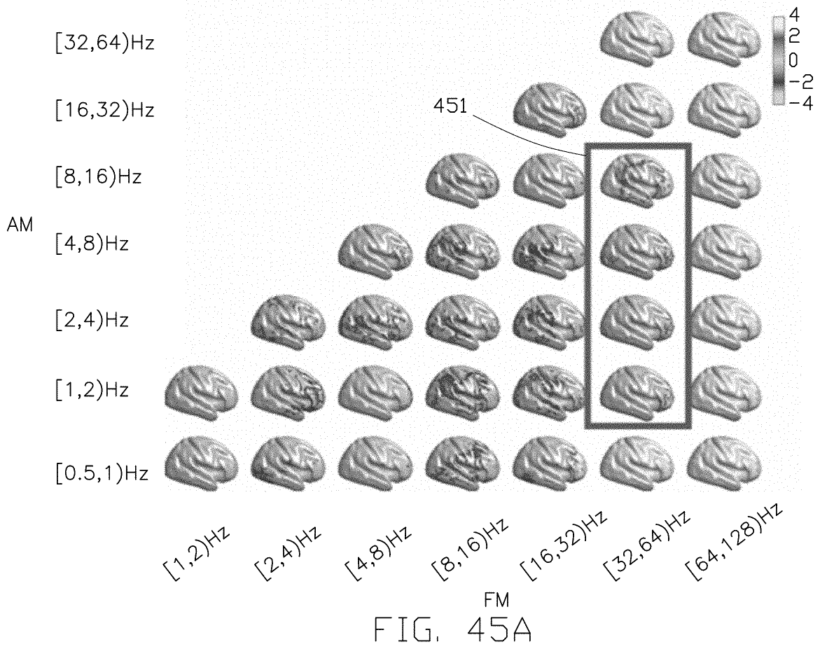

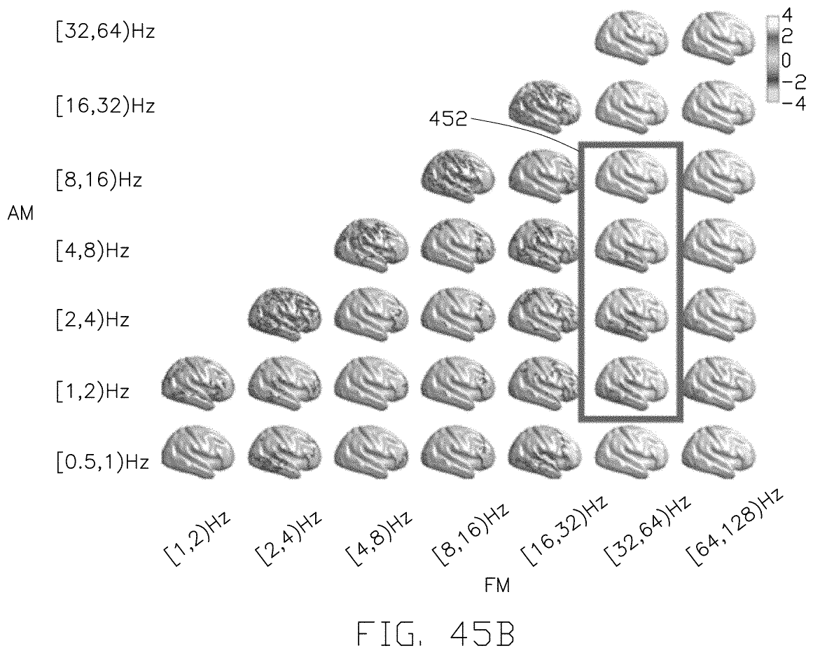

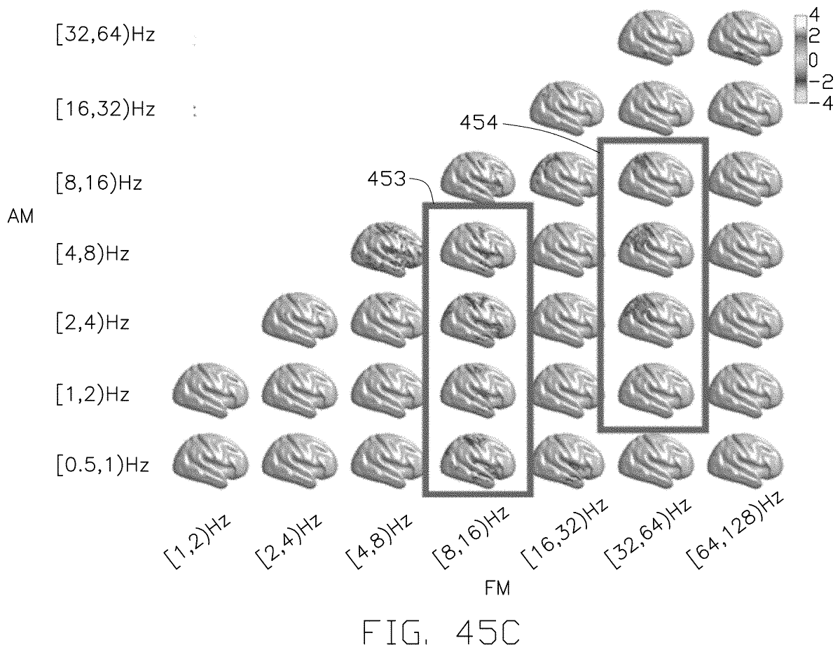

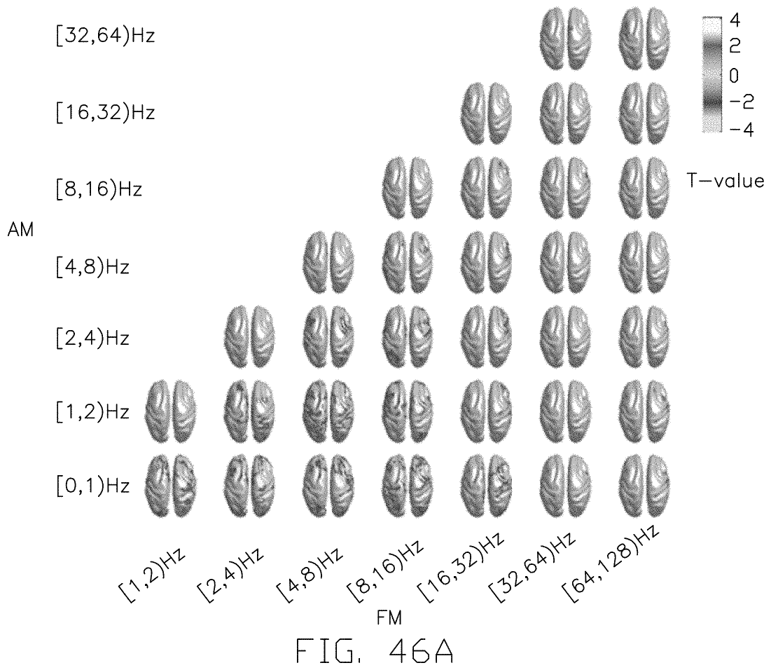

FIG. 45A, FIG. 45B, and FIG. 45C are marked-up visual outputs of the fEEToMGs comprising a plurality of tomographies of the brain on detection or diagnosis of HD, in accordance with embodiments of the present disclosure.

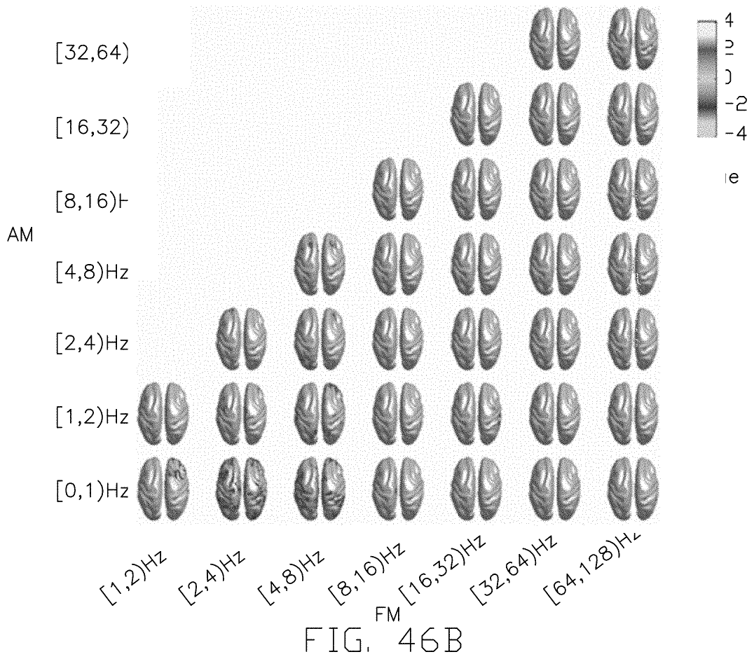

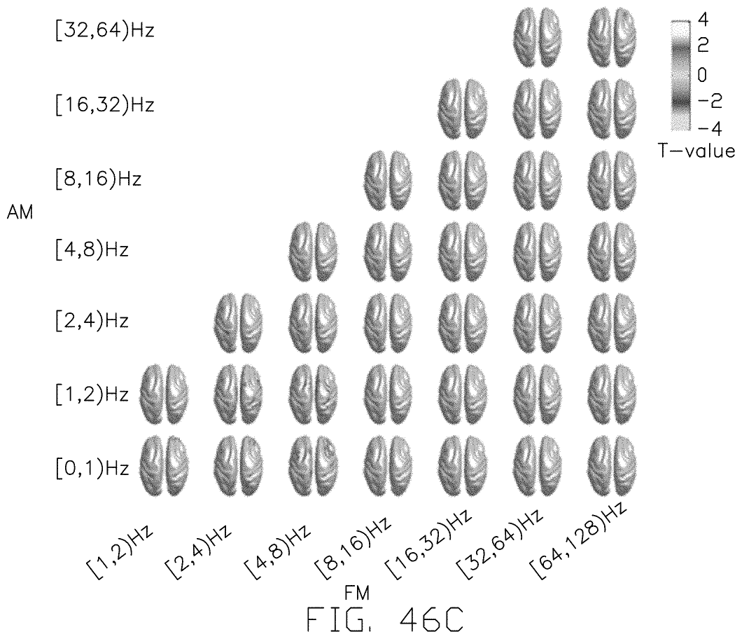

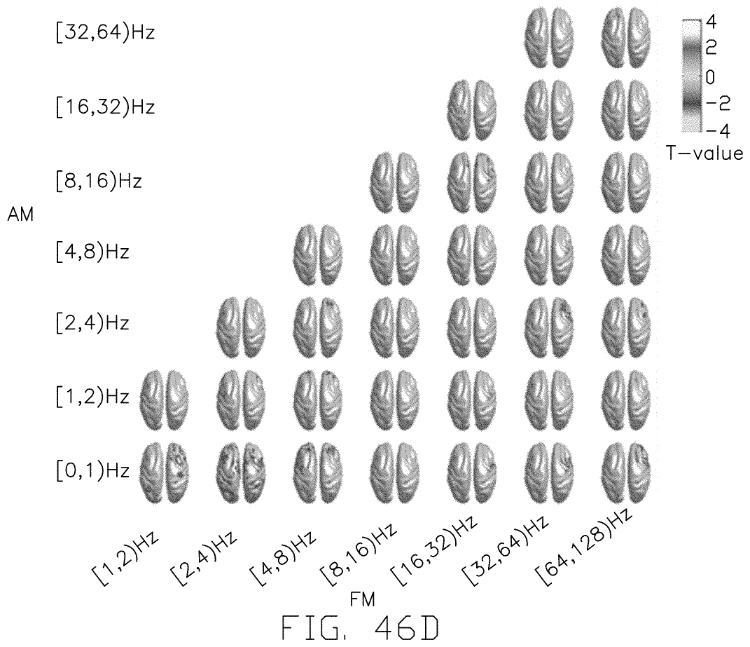

FIG. 46A, FIG. 46B, FIG. 46C, FIG. 46D, and FIG. 46E are visual outputs of the fEEToMGs comprising a plurality of tomographies of the brain on detection or diagnosis of migraine, in accordance with embodiments of the present disclosure.

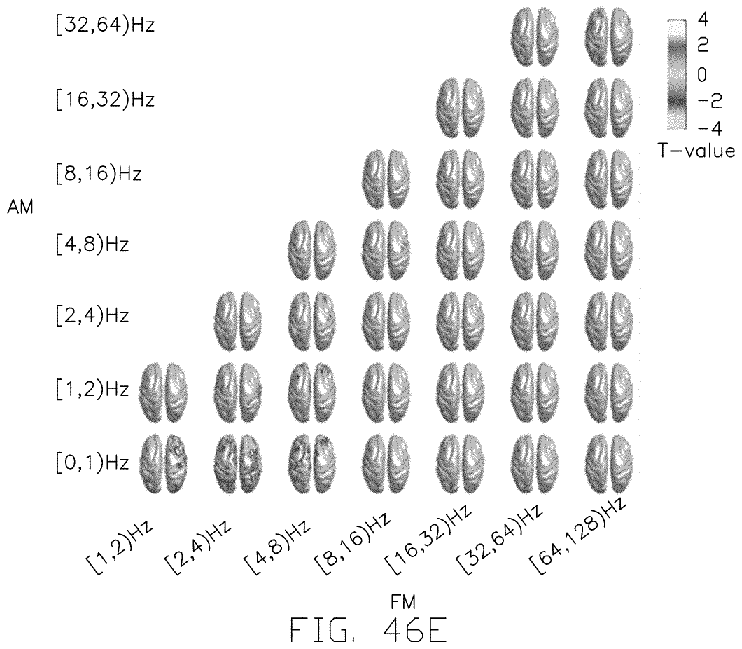

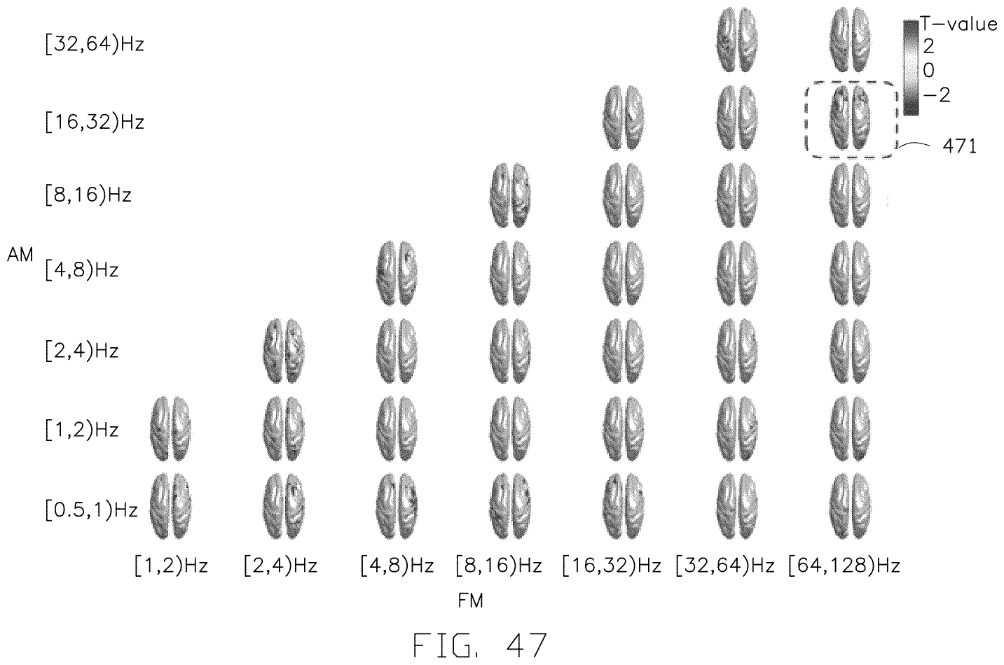

FIG. 47 is a marked-up visual output of the fEEToMG comprising a plurality of tomographies of the brain on detection or diagnosis of PD, in accordance with embodiments of the present disclosure.

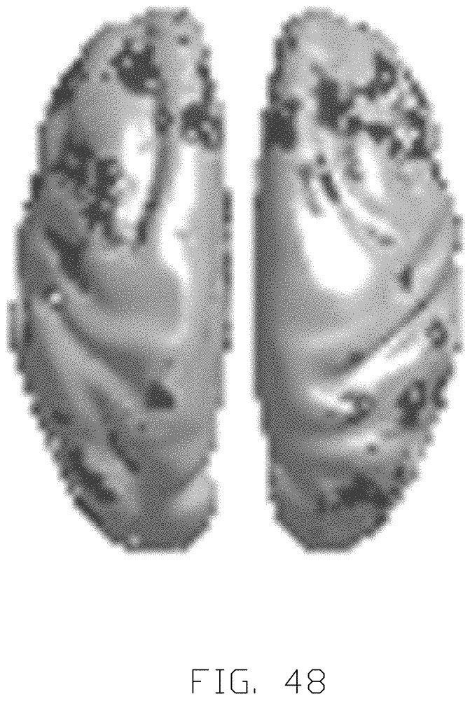

FIG. 48 is a visual output of the fEEToMG on detection or diagnosis of PD in accordance with an embodiment of the present disclosure.

### DETAILED DESCRIPTION

It will be noted at the beginning that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising,” when utilized, means “including, but not necessarily limited to”; it specifically indicates open-ended inclusion or membership in the so-described combination, group, series and the like.

Referring to FIG. 1, a system for analyzing electrical activities of the brain in accordance with an embodiment of the present disclosure is provided. The system 1 comprises a detection module 10, a transmission module 20, an analysis module 30 and a visual output module 40. The system 1 is configured to detect electrical activities of the brain, to analyze signals and to display graphical information of the analyzed results. The electrical activities of the brain may be represented by electroencephalography (EEG), magnetoencephalography (MEG), or electrocorticography (ECoG) signals. The system 1 may further comprise other electrical components or modules for better performance or user experience. For example, the system 1 may comprise an amplifier module or filter module to enhance signal to noise ratio by gaining signal strength within certain bandwidth and minimizing noise from environmental interference or baseline wandering. For example, the system 1 may comprise an analog-to-digital converter (ADC) for signal digitization. For example, the system 1 may further comprise a storage module for storing the digital signals or storing the analyzed data. In one example, the detection module 10 may further comprise a data acquisition module. The data acquisition module is capable of executing the functions of the amplifier module, ADC and the storage module. Furthermore, the system 1 may comprise a user input module for use to control the system 1, such as a keyboard, a mouse, a touch screen, or a voice control device.

The detection module 10 is configured to receive electrical activities of the brain and to convert the electrical activities into electrical signals. The detection module may be a transducer or a plurality of transducers converting the electrical activities of the brain into electrical signals. The brain electrical activities are the dynamic changes during the polarization and repolarization processes in neurotransmissions. When a transducer is applied on a region of the head, the transducer is able to detect the summation of the far field effects. The transducer may be a biopotential electrode to detect the electrical potentials or a magnetoelectric transducer to detect the magnetic fields. A ground electrode may be paired with the biopotential electrodes for measuring electrical potential differences and additionally a reference electrode may be presented for noise reduction. The detection module 10 may be applied on the surface of scalp to detect EEG or on the surface of the cortical brain to detect ECoG. In one example, the detection module 10 comprising an array of transducers may be arranged as a 10-20 system or other higher resolution systems. The biopotential electrodes could be wet (with saline water or conducting gels) or dry electrodes.

The detection module 10 may further comprise a data acquisition module. The data acquisition module may instruct a sampling rate to determine the time interval of the adjacent data points. The detected signal may be acquired and stored by a data acquisition module in the form of electrical potential (preferably measured by voltage) with corresponding temporal sequences.

The transmission module 20 is configured to receive the electrical signals from the detection module 10 and deliver the signals to the analysis module 30. The transmission module 20 may be wired or wireless. The wired transmission module 20 may include an electrical conductive material delivering the detected signal directly to the analysis module 30 or to the storage module for processing by the analysis module 30 thereafter. The detected signal may be stored in a mobile device, a wearable device or transmitted wirelessly to a data processing station through RF transmitters, Bluetooth, Wi-Fi or the internet. The mobile device can be a smartphone, a tablet computer, or a laptop. The wearable device can be a processor-embedded wristband, a processor-embedded headband, a processor-embedded cloth, or a smartwatch. The modules of the system 1 may be electrically coupled within a compact device or may be located discretely and coupled together by wired or wireless communication network.

The analysis module 30 is configured to process the signal by a series of action. The analysis module 30 may be a single microprocessor, such as a general purpose central processing unit, an application specific instruction set processor, a graphic processing unit, a field-programmable gate array, a complex programmable logic device or a digital signal processor. The analysis module 30 may execute a non-transitory computer program product embodied in the computer-readable medium. The analysis module 30 may comprise multiple microprocessors or processing units to execute the computer program product embodied in the computer-readable medium, in order to perform different functional blocks of the entire analysis process.

The visual output module 40 is configured to display the graphical results of the information generated by the analysis module 30. The visual output module 40 may be a projector, a monitor, or a printer for projecting the analysis results. In the embodiments, the analysis result is a visual output with graphic representations, and can be displayed by the visual output module 40 on a color monitor, be printed out on a paper or an electronic file, or be displayed on a grayscale monitor.

Referring to FIG. 2, a method for analyzing electrical activities of the brain in accordance with an embodiment of the present disclosure is provided. The method for analyzing the electrical activities of the brain may include the steps as mentioned below. The method comprises: detecting the electrical activities of the brain as a detected signal S21, performing empirical mode decomposition (EMD) on the detected signal to obtain a set of primary intrinsic mode functions (IMFs) S22, creating envelope functions of the corresponding of IMF S23a, performing EMD on the envelope functions to obtain sets of secondary IMF S24, performing a transformation on the plurality of primary IMFs to obtain the frequency modulation (FM) functions S23b, performing a transformation on the plurality of secondary IMFs to generate the AM function S25, generating data set according to the FM function and the AM function S26, generating a visual output space S27. The EMD in S22 can be complete ensemble empirical mode decomposition (CEEMD), ensemble empirical mode decomposition (EEMD), masking EMD, enhanced EMD, multivariate empirical mode decomposition (MEMD), noise-assisted multivariate empirical mode decomposition (NA-MEMD). The transformation in S23b and S25 can be Hilbert transform, Direct quadrature, inverse trigonometric function, or generalized zero-crossing. Detecting the electrical activities of the brain as a detected signal S21 is performed at the detection module 10. The analysis module 30 generates the analyzed data set from the detected signal and the analyzed data set may be stored in the computer-readable medium in the analysis module 30 for a scheduled display on the visual output module 40 thereafter. The analyzed data set comprises a plurality of analyzed data units.

The processes S22, S23a, S23b, and S25 are further elaborated in FIG. 3A to 3F, in accordance with an embodiment of the present disclosure. The detected signals are consequently transformed or decomposed into primary IMFs, secondary IMFs, envelope functions, AM functions, and FM functions.

Referring to FIG. 3A, a plurality of EMDs for detected signals are provided in accordance with an embodiment of the present disclosure. The detected signal is transformed into a set of primary IMFs by EMDs. The plurality of EMDs in FIG. 3A correspond to S22 of FIG. 2. The EMD is a process comprising a series of sifting process to decompose a signal into a set of IMFs. For example, a plurality of primary intrinsic functions is generated from the detected signal by EMD. A sifting process generates an intrinsic function from the detected signals. For example, a first sifting process generates a first primary IMF 31a from the detected signal 31; a second sifting process generates a second primary IMF 31b from the first primary IMF 31a; a third sifting process generates a third primary IMF 31c from the second primary IMF 31b; a mth sifting process generates a mth primary IMF 31n from the (m−1)th primary IMF 31m. The number of sifting processes is determined by stopping criteria. The stopping criteria may depend on the signal attenuation or the variation of the mth primary IMF 31n.

Furthermore, EMD may comprise masking procedure or noise (even pairs of positive and negative values of the same noise) addition procedure with variable magnitude adapted for each sifting step to solve mode mixing problems. EMD may be achieved by ensemble techniques.

Referring to FIG. 3B, a plurality of interpolation processes is provided in accordance with an embodiment of the present disclosure. The interpolation processes in FIG. 3B correspond to S23a in FIG. 2. An envelope function is the interpolation function generated by an interpolation process from detected signals. The envelope function connects local extrema of the detected signals. Preferably, the envelope connects local maxima of the absolute-valued function of the detected signals. The interpolation process may be achieved via linear interpolation, polynomial interpolation, trigonometric interpolation or spline interpolation, preferably cubic spline interpolation. The envelope functions in FIG. 3B are generated from IMFs in FIG. 3A by the interpolation processes. A first envelope function 32a may be generated from the first primary IMF 31a; a second envelope function 32b may be generated from the second primary IMF 31b; a third enveloped function 33b may be generated from the third primary IMF 31c; a (m−1)th envelope function 32m may be generated from the (m−1)th primary IMF 31m; a mth envelope function 32n may be generated from the nth primary IMF 31n.

Referring to FIG. 3C, a plurality of EMDs is provided in accordance with an embodiment of the present disclosure. The plurality of sets of secondary intrinsic functions are generated from the envelope functions by EMD. The EMDs in FIG. 3C correspond to S24 in FIG. 2. The first set of secondary IMFs 33a is generated from the first envelope function 32a; the second set of secondary IMFs 33b is generated from the second envelope function 32b; the (m−1)th set of the plurality of secondary IMFs 33m is generated from the (m−1)th envelope function 32m; the mth set of the plurality of secondary IMFs 33n is generated from the mth envelope function 32n.

Referring to FIG. 3D, a plurality of sets of secondary IMFs are provided in accordance with an embodiment of the present disclosure. The mth envelope function 32n, the mth set of secondary IMFs 33n, and the secondary IMFs included in the mth set of secondary IMFs 33n are illustrated in FIG. 3D. The mth envelope function 32n in FIG. 3B comprises a first secondary IMF 34a of the mth set of secondary IMFs 33n, a second secondary IMF 34b of the mth set of secondary IMFs 33n, a third secondary IMF 34c of the mth set of secondary IMFs 33n, a (n−1)th secondary IMF 34m of the mth set of secondary IMFs 33n, and a nth secondary IMF 54n of the mth set of secondary IMFs 33n. Therefore, there are IMFs in a number of m (number of the plurality of sets of secondary IMF) multiplying n (number of individual secondary IMFs in a set of secondary IMF in FIG. 3D.

Referring to FIG. 3E and FIG. 3F, a series of transformation processes is provided in accordance with an embodiment of the present disclosure. The transformation process is to convert a function from real domain to complex domain. The transformation process comprises at least a transformation and a complex pair function formation. The transformation process may be a Hilbert transform, a direct-quadrature-zero transform, an inverse trigonometric function transform, or a generalized zero-crossing transform. The complex pair function formation is to combine the function as the real part of the complex pair function and the transformed function as the imaginary part of the complex pair function.

In FIG. 3E, the FM functions are the complex pair functions generated from the plurality of primary IMFs by a proper transformation process. The transformation processes in FIG. 3E correspond to S23b in FIG. 2. The first primary IMF 31a is transformed into a first FM function 35a by the transformation process; the second primary IMF 31b is transformed into a second FM function 35b by the transformation process; the third primary IMF 31c is transformed into a third FM function 35c by the transformation process; and the mth primary IMF 31n is transformed into a mth FM function 35n by the transformation process.

In FIG. 3F, the AM functions are the complex pair functions generated from the secondary IMFs by a series of transformation processes. The transformation processes in FIG. 3F correspond to S25 in FIG. 2. The first secondary IMF 34d of the first set of secondary IMFs may be transformed into a (1,1) AM function 36d by the transformation process; the second secondary IMF 34e of the first set of secondary IMFs is transformed into a (1,2) AM function 36e by the transformation process . . . and the nth secondary IMF 34k of the first set of the secondary IMFs is transformed into a (1, n) AM function 36k by the transformation process. Furthermore, the nth secondary IMF 34n of the mth set of secondary IMFs may be transformed into a (m, n)th AM function 36n by the transformation process.

Referring to FIG. 4, elements of an analyzed data unit is provided in accordance with an embodiment of the present disclosure. In FIG. 4, the analyzed data unit 41 comprises a time period 42, a first coordinate 43, a second coordinate 44 and a signal strength value 45. In one embodiment, the time period 42 is a period of time when the detection module detects the physiological signals, the first coordinate 43 indicates instantaneous frequency of FM measured by frequency (Hertz), and the second coordinate 44 indicates instantaneous frequency of AM measured by frequency (Hertz). The signal strength value 45 may indicate signal amplitude measured by electrical potential (voltage) or electrical current (ampere) or may indicate signal energy measured by energy strength per unit time interval (watt). For each analyzed data unit within the time period, the first coordinate 43 can be the argument of the mth FM functions 35n in FIG. 3E at corresponding time period; the second coordinate 44 can be the argument of the (m, n)th AM function 36n in FIG. 3F at corresponding time period; the signal strength value 35 is the value of the envelope function at corresponding time period. Preferably, the second coordinate 44 is larger than the first coordinate 43.

Referring to FIG. 5, a schematic visual output from a plurality of analyzed data sets is provided in accordance with an embodiment of the present disclosure. In FIG. 5, the visual output 5 comprises a first axis 53, a second axis 54 and a plurality of other visual elements 51a-51f. The first axis 53 can be a frequency scale of FM, or a logarithmic scale of FM. The second axis 54 can be a frequency scale of AM, or a logarithmic scale of AM. Each of the visual elements 51a-51f comprises an analyzed data set and an accumulated signal strength. Each of the analyzed data set is an integral of a plurality of analyzed data units, therefore each of the visual element 51a-51f comprises multiple analyzed data units within a certain range of the FM frequency and the AM frequency in a time period. The accumulated signal strength of each of the visual elements 51a-51f is an integral of the signal strengths of each of the analyzed data units. For instance, the accumulated signal strength of the visual element 51e is an integral of the signal strengths of the analyzed data unit a 52a, the analyzed data unit b 62b, the analyzed data unit c 52c, and the analyzed data unit d 52d. The accumulated signal strength can be presented by color scale, dot density, grayscale, or screetones, wherein different colors, dot densities, grayscales, or screetones indicate different values of the accumulated signal strength (not shown). The visual output module 40 in FIG. 1 renders a visual output space according to the analyzed data sets, and displays the visual output 5.

A smoothing process may be applied to the visual output space for the visual elements with sparse data units. For example, the smoothing process may be Butterworth filter, exponential smoothing, Kalman filter, Kernal smoother, Laplacian smoothing, moving average, or other image smoothing techniques.

Following the methods, principles and transformation processes illustrated in FIG. 2, FIG. 3A-3F, FIG. 4 and FIG. 5, a plurality of embodiments from detected physiological signals are demonstrated in FIG. 6A-6D, FIG. 7, FIG. 8 FIG. 9A-9B, and FIG. 10.

The detected signal and the IMFs generated via EMD process are shown in FIG. 6A-6D in accordance with an embodiment of the present disclosure. In some embodiments, FIG. 6A-6D are intermediate outputs from the detected electrical activities of the brain. In FIG. 6A, the detected signal stored as the detected data set is plotted along time. FIG. 6B shows a plurality of primary IMFs generated from the detected signal by EMD. FIG. 6C shows the first set of secondary IMFs generated from the first envelope function. FIG. 6D shows the second set of secondary IMFs generated from the second envelope function.

Referring to FIG. 7, a visual output for the analyzed data units are provided in accordance with an embodiment of the present disclosure. The analyzed data units are plotted in a three-dimensional space comprising AM axis, FM axis, and time axis. The signal strength of each of the analyzed data units is also given but not shown in the visual output. Each plotted dot is an analyzed data unit. An integral of analyzed data units within a time period is an analyzed data set.

Referring to FIG. 8, a heat map transformed from the plot graph of FIG. 7 is provided in accordance with an embodiment of the present disclosure. The heat map is a form of the visual output. The heat map comprises an axis of FM and an axis of AM. In the embodiment, each visual element comprises an analyzed data sets and an accumulated signal strength, which is an integral of all the signal strength of the analyzed data units within a time period. In other words, the time axis of FIG. 7 is deducted in FIG. 8. As illustrated in FIG. 8, the grayscale represents the accumulated signal strength of each of the visual elements, or blocks, and may have different shades of gray proportional to the accumulated signal strength: a dark gray or black to represent the smallest accumulated signal strength, a lighter gray surrounded by a darker gray to represent an intermediate accumulated signal strength, and a dark gray surrounded by a lighter gray to represent the largest accumulated signal strength. For instance, an area 81 is a combination of an area with 0-0.3 Hz of FM and 0.01-0.02 Hz of AM and another area with 0-0.1 Hz of FM and 0-0.01 Hz of AM. The area 81 is an area of dark gray surrounded by lighter gray, so the area 81 has the largest accumulated signal strength in FIG. 8. Conversely, the grayscale may also use a dark gray surrounded by a lighter gray to represent the smallest accumulated signal strength, and a dark gray or black to represent the largest accumulated signal strength in some embodiments.

Additionally, the accumulated signal strength in the heat map may be represented by a color scale, dot density, or screentone. In one embodiment, the dot density may be higher for a larger accumulated signal strength, and lower density for a smaller accumulated signal strength. In another embodiment, the color scale may use blue to indicate the smallest accumulated signal strength, green to indicate an intermediate accumulated signal strength, and yellow, orange, or red to indicate the largest accumulated signal strength. The color scale may also include a color transition from one color to another color, such as the color transition from blue to green or from orange to red. In still another embodiment, the screentone with more grids may represent larger accumulated signal strength, and the screentone with more dots may represent lower accumulated signal strength. Conversely, the color scale, dot density, or screentone can have different meanings for different colors, dot densities, contour lines, or screentones for various levels of the accumulated signal strength.

The dot densities in dot density graph, different shades of gray in grayscale, various colors in the color scale, the densities of contour lines, and different screentones in the visual output indicate the accumulated signal strength by the analyzed data unit, and they may represent a relative or an absolute scale of the accumulated signal strength. The visual output space may be rendered dynamically along with sliding time periods so that the visual output module is capable of displaying the HOSA spectrum not only as a graph, but as a video.

Referring to FIG. 9A and FIG. 9B, visual outputs of a logarithmic scale of AM axis and FM axis of the analyzed data unit are provided in accordance with embodiments of the present disclosure. In FIG. 9A, the X-axis is a logarithmic scale of FM, and the Y-axis is a logarithmic scale of AM. The accumulated signal strengths in FIG. 9A is indicated by a grayscale, which has similar meanings as the grayscale in FIG. 8. An area 91 and another area 92 have the largest accumulated signal strengths in FIG. 9A. The area 91 is generally located in Log 2 FM=0 and Log 2 AM=−6 to −4, and the area 92 is generally located in Log 2 FM=0-5 and Log 2 AM=−4 to −2. Preferably, the base of the logarithmic scale is assigned as 2 because of the dyadic property of EMD process. In FIG. 9B, the HOSA spectrum may be plotted with contour lines, with higher density of the contour lines representing larger accumulated signal strength. For instance, an area 93, an area 94, and an area 95 have the largest accumulated signal strengths in FIG. 9B. The area 93 is generally located in 4 Hz of AM and 8-16 Hz of FM. The area 94 is generally located in 2 Hz of AM and 8-16 Hz of FM. The area 95 is generally located in 1 Hz of AM and 8-16 Hz of FM. The contour lines may also be combined with dot density, color scale, or screentone to indicate various levels of the accumulated signal strength.

Referring to FIG. 10, another visual output of an AM axis and FM axis with enhanced contrast lines is provided in accordance with an embodiment of the present disclosure. In FIG. 10, the visual elements, or the blocks, may represent the difference between an analyzed data unit and a reference data unit, therefore FIG. 10 can be used to compare two different visual outputs, and the difference between a reference data unit and an analyzed data unit can be indicated by different colors, grayscales, dot densities, or screentones. The contrast lines in FIG. 10 may be processed by a normalization process to align with a linear scale or a distribution model, such as normal distribution. The reference data units are used as control group data, and may be generated from a standard data unit or a longitudinal data unit. The standard data unit is generated from an average of the analyzed data units from a specific group of subjects. For example, the specific group of subjects may be healthy subjects or subjects diagnosed without a particular disease state. To eliminate the individual variations, normalization of the individual data can be used. The longitudinal data unit is an experiment group, and may be generated from the previous analyzed data units of the same subject. In some embodiments, z-score or t-score can be calculated according to the two data sets. The device may further generate a graph to demonstrate that the location of the analyzed data unit in a distribution model.

The visual elements in FIG. 10 may also represent an analyzed data set and an accumulated signal strength, therefore in this scenario the accumulated signal strength can be indicated by different colors, grayscales, dot densities, or screentones.

The visual output of the analyzed data set of electrical activities of the brain can be used to compare 2 or more states of different groups of people, different individuals, or the same individual. The visual output can be the heat map as in FIG. 8, the logarithmic graph of AM and FM as in FIG. 9A-9B and the non-logarithmized AM-FM graph of FIG. 10. Specific visual output patterns of one or more particular diseases can be identified. The specific visual output patterns may comprise a disease state, a healthy state, a good prognosis state, a poor prognosis state, or other patterns relevant to diagnosis, prognosis, clinical evaluation, or staging of the disease. The comparison between the specific visual output patterns may be used to identify the difference between two groups of people with different mental condition, two groups of people with different disease stage, two groups of people with different prognosis of the disease, two individuals with different mental condition, two individuals with different disease stage, two individuals with different prognosis of the disease, or two different time periods of the same individual. The comparison on specific patterns may lead to establish a model for the clinical evaluation, diagnosis, staging or prognosis of the disease.

A healthy state could be defined as a subject or a group of subjects without being diagnosed with particular disease(s) of interest. A disease state could be defined as a subject or a group of subject being diagnosed with particular disease(s) of interest. The healthy state and the disease state may be presented on the same subject on different time periods or be presented on different subjects.

The present disclosure will now be described more specifically with reference to the following exemplary embodiments, which are provided for the purpose of demonstration rather than limitation.

1. Functional Electroencephalotopography (fEEToPG)

Referring to FIGS. 11 and 12, visual outputs of functional electroencephalotopography (fEEToPG) are provided in accordance with embodiments of the present disclosure. In a system for generating the fEEToPG, the detection module comprises an array of detection units to synchronously detect brain electrical activities of different regions of a scalp. The array of detection units may be electrodes arranged in a specific spatial pattern, wherein each electrode obtains a detected signal of a corresponding region.

The method of visualization of the fEEToPG comprises: detecting brain electrical activity by the detection module; generating a plurality of analyzed data sets from a plurality of detection units, and each of the analyzed data sets comprising a plurality of analyzed data units, and each of the analyzed data unit further comprising a detection unit ID; modeling signal strength values for each of the visual elements between the detection units within a boundary of a two-dimensional anatomical graph, and rendering an anatomical graph from the detection unit IDs. Each of the detection unit ID comprises anatomical information of a corresponding region for a detection unit. The visual elements are areas within the boundary in the anatomical graph and comprises signal strength values of the analyzed data units. Some visual elements represent the location of the detection unit IDs, but some other visual elements are intermediate areas not covered by, and between the detection unit IDs. The modeling process determines modeled signal strength values for the intermediate areas not covered by the detection unit IDs, and the modeling process may be achieved by interpolation between the accumulated signal strength of the detection unit IDs. The intermediate areas often have modeled signal strengths that are transitional between the accumulated signal strengths of two adjacent detection unit IDs. The anatomical graph, or the topography, can be a two-dimensional anatomical graph depicting a two-dimensional brain structure marked with the locations of multiple detection units.

FIG. 11 shows an example of a topography 11 generated by the HOSA method in accordance with an embodiment of the present disclosure. In FIG. 11, the detection module is a 10-20 system comprising 21 detection units, and each of the detection units in FIG. 11 is indicated. Within a particular amplitude modulation (AM) frequency and frequency modulation (FM) frequency, each of the visual elements located on the detection units has an accumulated signal strength value, and each of the visual elements not covered by the detection unit also has a modeled signal strength value. The modeling process determines the signal strength values for the visual elements not covered by and between the detection units, and the modeling process may be achieved by various source localization methods. For example, independent component analysis or beam forming methods can be applied for modeling process. Each of the visual elements in the topography 11 comprise an accumulated signal strength of an analyzed data unit, or a modeled signal strength within a particular AM frequency and a FM frequency. The accumulated signal strengths and modeled signal strengths in FIG. 11 are indicated by different shades of grayscales: a dark gray or black to represent the smallest signal strength, a lighter gray surrounded by a darker gray to represent an intermediate signal strength, and a dark gray surrounded by a lighter gray to represent the largest signal strength. In FIG. 11, an area 111 has the largest signal strength, and an area 112, 113, and 114 have the smallest signal strengths.

FIG. 12 shows an example of the fEEToPG comprising a plurality of topographies in accordance with embodiments of the present disclosure. The visual output of the fEEToPG in FIG. 12 comprises a plurality of topographies in different AM frequencies and FM frequencies. In FIG. 12, the X-axis is a FM frequency scale and the Y-axis is an AM frequency scale. Each of the topographies in the visual output 12 has a AM frequency and a FM frequency, and is plotted according to the AM-axis and the FM-axis. The grayscales on each of the topographies in the visual output 12 represent different accumulated signal strengths or modeled signal strengths, and have similar meaning with the grayscales in FIG. 11. The visual output 12 represents various accumulated signal strengths in different AM-FM frequency on different locations of the scalp.

The visual output in FIG. 12 may also be used to demonstrate the differences between two sets the fEEToPGs from different groups of subjects, different subjects, or the same subjects on different time periods. The visual elements may comprise t-score, z-score, or power levels to indicate the differences between two sets of the fEEToPGs. The differences may be indicated by various colors, grayscales, dot densities, or screentones.

2. Functional Electroencephalotomography (fEEToMG)

Referring to FIG. 13-16, visual outputs of functional electroencephalotomography (fEEToMG) are provided in accordance with embodiments of the present disclosure. In a system for generating the fEEToMG, the detection module comprises an array of detection units to synchronously detect brain electrical activities of different regions of a scalp. The array of detection units may be electrodes arranged in a specific spatial pattern, wherein each electrode obtains a detected signal of a corresponding region.

The method of visualization of the fEEToMG comprises: detecting brain electrical activity by the detection module; generating a plurality of analyzed data sets from a plurality of detection units, and each of the analyzed data sets comprising a plurality of data units, and each of the analyzed data unit further comprising a detection unit ID; modeling signal strength values for each of the visual elements between the detection units within a boundary of three dimensional anatomical model, and rendering the anatomical model from the detection unit IDs. Each of the detection unit ID comprises anatomical information of a corresponding region for a detection unit. The visual elements are areas within the boundary in the anatomical model and comprises signal strength values of the analyzed data units. Some visual elements represent the location of the detection unit IDs, but some other visual elements are intermediate areas not covered by the detection unit IDs. The modeling process determines modeled signal strength values for the intermediate areas not covered by, and between the detection unit IDs, and the modeling process may be achieved by various source localization methods. For example, independent component analysis or beam forming methods can be applied for modeling process. The intermediate areas often have modeled signal strengths that are transitional between the accumulated signal strengths of two adjacent detection unit IDs. The anatomical model, or the tomography, can be a three-dimensional anatomical model depicting a three-dimensional brain structure.

FIG. 13 shows a tomography 13 generated by the HOSA method in accordance with an embodiment of the present disclosure. In FIG. 13, the tomography 13 is a three dimensional anatomical model. Within a particular amplitude modulation (AM) frequency and frequency modulation (FM) frequency, each of the visual elements located on the detection units has an accumulated signal strength value, and each of the visual elements not covered by the detection unit also has a modeled signal strength. Each of the visual elements covered by the detection unit IDs in the tomography 13 comprise an accumulated signal strength value of an analyzed data unit within a particular AM frequency and a FM frequency. The accumulated signal strengths and the modeled signal strengths in FIG. 13 are indicated by different grayscales. In FIG. 13, an area 131 has the largest accumulated signal strength and is generally located on the temporal lobe of the tomography 13.

FIG. 14A-14D show the tomographies generated by the HOSA method in accordance with embodiments of the present disclosure. FIG. 14A-14D are generated via similar modeling process as in FIG. 13. FIG. 14A shows a left lateral view of a tomography 141 generated by the HOSA method, and an area 1411 has the largest accumulated signal strength and is generally located on the temporal lobe of the tomography 141. FIG. 14B shows a left medial view of a tomography 142 generated by the HOSA method, and an area 1421 has the largest accumulated signal strength and is generally located on the parietal lobe of the tomography 142. FIG. 14C shows a right lateral view of a tomography 143 generated by the HOSA method, and an area 1431 has the largest accumulated signal strength and is generally located on the intersection of the occipital lobe and the parietal lobe. FIG. 14D shows a right medial view of a tomography 144 generated by the HOSA method.

FIG. 15A-FIG. 15C show MRI images modeled with tomographies generated by the HOSA method in accordance with embodiments of the present disclosure. FIG. 15A shows a coronal view of an MRI image modeled with a tomography generated by the HOSA method, and a location 151 having the largest accumulated signal strength is marked on the MRI image 151. FIG. 15B shows a sagittal view of an MRI image modeled with a tomography generated by the HOSA method, and a location 152 having the largest accumulated signal strength is marked on the MRI image. FIG. 15C shows a transverse view of an MRI image 153 modeled with a tomography generated by the HOSA method, and a location 1531 having the largest accumulated signal strength is marked on the MRI image 153. The three dimensional anatomical model may be generated from other medical imaging modalities, such as a computer tomography (CT) image or a sonographic image.

FIG. 16 shows an example of the fEEToMG comprising a plurality of tomographies in accordance with embodiments of the present disclosure. The visual output 16 of the fEEToMG in FIG. 16 comprises the tomographies in different AM frequencies and FM frequencies. In FIG. 16, the X-axis is a FM frequency scale and the Y-axis is an AM frequency scale. Each of the tomographies in the visual output 16 has a AM frequency and a FM frequency, and is plotted according to the AM-axis and the FM-axis. The grayscales on each of the tomographies in FIG. 16 represent different accumulated signal strength, and have similar meaning with the grayscales in FIG. 13. FIG. 16 represents various accumulated signal strength in different AM-FM frequency on different locations of the brain.

The visual output in FIG. 16 may also be used to demonstrate the differences between two sets the fEEToMGs from different groups of subjects, different subjects, or the same subjects on different time periods. The visual elements may comprise t-score, z-score, or power levels to indicate the differences between two sets of the fEEToMGs. The differences may be indicated by various colors, grayscales, dot densities, or screentones.

3. Clinical Applications of Functional Electroencephalography (fEEG)

Clinical applications of functional electroencephalography (fEEG) is illustrated in the following figures to identify specific visual output patterns among various types of neuropsychiatric disorders, for example, cognitive function impairment, Alzheimer's disease, Huntington disease, depression, migraine, anesthesia depth, insomnia, Parkinson's disease, attention deficit hyperactivity disorder (ADHD), depth of anesthesia monitoring, and drug addiction. The specific visual output patterns of the fEEG is relevant to diagnosis, prognosis, clinical evaluation, or staging of the above diseases. The comparison between specific visual output patterns of the fEEG may be used to identify the difference between two groups of people with different mental condition, two groups of people with different disease stage, two groups of people with different prognosis of the disease, two individuals with different metal condition, two individuals with different disease stage, two individuals with different prognosis of the disease, or two different time periods of the same individual.

3.1 The fEEG of Alzheimer's Disease (AD)

Alzheimer's disease (AD) is a neuro-degenerating diseases and is one of the most debilitating and devastating diseases for an individual and one's family. It is also the most financially costly disease for the ageing society in the world. According to the United Nations, there were 26.6 million diagnosed with AD in 2006, and it is every 1 in 85 million people will be affected by 2050. AD is the cause of 60% to 70% of all dementia patients and is definitely relevant to age. Therefore, AD along could potentially break the health insurance system of many countries. Although there is a genetic factor (Familia AD) accounting for about 5-10% of all AD cases, the majority (90-95%) of the cases are episodic ADs.

A definitive diagnosis of AD is difficult. Conventional diagnosis of AD is based on behavioral observations and medical history of an individual or the family. Most commonly used criteria for AD diagnosis are established by the National Institute of Neurological and Communicative Disorders and Stroke (NINCDS) and the Alzheimer's Disease and Related Disorders Association (ADRDA). The criteria listed 8 cognitive domains that are most commonly impaired in AD such as memory, language, perceptual skills, attention, constructive abilities, orientation, problem solving and functional abilities. Unfortunately, the above criteria or most of the conventional medical imaging techniques are not sensitive in early stage of AD. Definitive diagnosis has to be made by pathology from autopsy of the brain.

Referring to FIG. 17A-FIG. 17C, the fEEGs for detection or diagnosis of dementia are provided in accordance with embodiments of the present disclosure. FIG. 17A-17C are similar to the visual output of FIG. 10, whereby the X-axis is a scale of FM frequency and the Y-axis is a scale of AM frequency, and wherein each of the visual elements may represent the difference between an analyzed data unit and a reference data unit. The analyzed data units in FIG. 17A-17C are subjects diagnosed with clinical dementia rating (CDR). The reference data unit in FIG. 17A-17C are normal subjects without any CDR rating. the fEEGs of FIG. 17A-17C are the results of an EEG electrode “Pz” channel of the 10-20 system. FIG. 17A compares the EEGs of MCI (Mild Cognitive Impairment) subjects with normal subjects. FIG. 17B compares the EEGs of CDR1 subjects with the normal subjects. FIG. 17C compares the EEGs of CDR2 subjects with the normal subjects. The white contour lines in FIG. 17A-17C indicate one or more areas wherein P-value<0.005.

Referring to FIG. 18A-18C, the fEEGs for detection or diagnosis of dementia are provided in accordance with embodiments of the present disclosure. FIG. 18A-18C are similar to the visual outputs of FIG. 17A-17C, and are also the results of an EEG electrode “Pz” channel of the 10-20 system. FIG. 18A compares the EEGs of CDR1 subjects with MCI subjects. FIG. 18B compares the EEGs of CDR2 subjects with CDR1 subjects. FIG. 18C compares the EEGs of CDR3 subjects with CDR2 subjects. The reference data units are MCI subjects in FIG. 18A, CDR1 subjects in FIG. 18B, and CDR2 subjects in FIG. 18C, respectively.

FIG. 18A-18C do not use normal subjects as the reference data sets. Therefore, there are more differences between CDR subjects and normal subjects, than the differences within CDR subjects.

Referring to FIG. 19A, the fEEG for comparing CDR1 and CDR0.5 is provided in accordance with embodiments of the present disclosure. FIG. 19A is the results of paired t-test (within-subjects), and are similar to the visual output of FIG. 18A-18C, but both “Cz” and “Pz” channels of the 10-20 systems are used. There are 26 subjects in FIG. 19A and are classified by CDR, and the differences between the CDR1 subjects and CDR0.5 subjects are shown in FIG. 19A. The white contour lines in FIG. 19A indicates one or more areas wherein P-value<0.05.

Referring to FIG. 19B, the fEEG for comparing CDR2 and CDR1 is provided in accordance with embodiments of the present disclosure. FIG. 19B are similar to the visual output of FIG. 19A. There are 26 subjects in FIG. 19B and are classified by CDR, and the differences between CDR2 subjects CDR1 subjects are shown in FIG. 19B.

Referring to both FIGS. 19A and 19B, the differences between two groups of data in FIG. 19A are more than in FIG. 19B, this is indicated by the white contour lines in FIG. 19A. Therefore, there are more differences between CDR1 subjects and CDR0.5 subjects, than between CDR1 subjects and CDR2 subjects. The visual output provided in FIG. 17A-17C, FIG. 18A-18C, FIG. 19A-19B can be visualized references for the diagnosis or staging of AD.

3.2 The fEEG of Huntington's Disease (HD)

Huntington's disease (HD) is an inherited autosomal dominant disorder that is characterized by disturbances in movement, cognition, memory and personality. The result of this disease is selective neuronal cell death occurring primarily in the cortex and striatum. After over two decades of investigation, the pathogenesis and pathological hallmarks of HD are well characterized. However, there is no therapeutic intervention currently available to delay or cure HD. One major hurdle that slows the development of drugs or interventions in HD is due to the lack of biomarkers that can be used (1) to track the disease progression (especially in the pre-manifest HD stage), and (2) as the primary end-point to validate the efficacy of intervention within a short period of time (e.g. 6-12 months).

As a progressive disorder, the change or improvement of behavioral symptoms in HD patents may take years to observe. Therefore, it is important to develop a non-invasive method that detects the change of brain functions prior to the appearance of behavior/symptoms, and track the progress of the changes.

Currently, the diagnosis of HD is based on family history and genetic tests, which gives a probability prior to the appearance of symptoms. Unfortunately, this life changing pre-clinical genetic test carries significant impact on the patient's psychology, career, family, and relationships. As a result, only a small percentage of those at risk choose to undergo the procedure. Additional physical and psychological examinations couple with family history and genetic test can help to determine the onset of HD. Unfortunately, the symptoms are usually only recognized in hindsight when HD symptoms are further developed. Therefore, a non-invasive, quantitative tool to determine the onset of HD is needed.

Referring to FIG. 20A and FIG. 20B, the fEEGs for detection or diagnosis of HD are provided in accordance with embodiments of the present disclosure. FIGS. 20A and 20B are similar to the visual output of FIG. 10, whereby the X-axis is a scale of FM frequency and the Y-axis is a scale of AM frequency, and wherein each of the visual elements may represent the difference between an analyzed data unit and a reference data unit. 15 HD patients in FIG. 20A and 5 pre-HD carriers in FIG. 20B are the analyzed data units, and 27 non-HD subjects are the reference data units in FIGS. 20A and 20B. FIGS. 20A and 20B are the results of two EEG electrode “O1” and “O2” channels of the 10-20 system. Abnormalities in the fEEG were observed when comparing pre-HD carriers with normal subjects in FIG. 20A, and HD subjects with normal subjects in FIG. 20B. The differences are statistically significant, in which the white line contour indicates statistical significance with P-value<0.05.

3.3 The fEEG of Depression

Major depressive disorder is one of the most common mental disorders in the United States: 16% of the U.S. population experienced depression during some period in their lives. Depression could also cause numerous comorbidities and contribute to higher mortality rates in cardiovascular diseases and many other conditions. It is also the leading cause for suicides, and the main component for Post-traumatic stress symptom. Conventional diagnosis of depression is based behavior of the subject. Clinically, only questionnaires and checklists (such as the Beck Depression Inventory or the Children's Depression Inventory) can be used by mental health providers to help detect and assess the severity of depression. Semi structured interviews such as the Kiddie Schedule for Affective Disorders and Schizophrenia (KSADS) and the Structured Clinical Interview for DSM-IV (SCID) are also used for diagnostic confirmation of depression. There is no definitive evidence based diagnosis available. A non-invasive, definitive diagnosis of the depression is needed.

Referring to FIG. 21A and FIG. 21B, the fEEG for detection or diagnosis of depression or mood disorder are provided in accordance with embodiments of the present disclosure. FIGS. 21A and 21B are similar to the visual output of FIG. 8, whereby the X-axis is a scale of FM frequency and the Y-axis is a scale of AM frequency. FIGS. 21A and 21B are the results of an EEG electrode “Pz” channel of the 10-20 system. the fEEG of a heathy subject is shown in FIG. 21A, and an area 211 has the largest accumulated signal strength. The fEEG of a major depression patient is shown in FIG. 21B, and an area 212 has the largest accumulated signal strength.

3.4 The fEEG of Migraine Headache

The typical symptoms for migraine headache is severe pulsating pain affecting one side of the head. Each incidence could last from 2 to 72 hours. Occasionally, the headache is also accompanied by nausea, vomiting. The pain could be aggravated by light, sound and smell. Some patient had experienced aura consisting of sight, sound or even strange smell, before the onset of the head attack, but not all patients experience aura.

Currently, diagnosis of migraine headache is based on signs and symptoms reported by the patients. Medical imaging techniques are not indicated for migraine, but may be used to rule out of other causes of headaches. Therefore, there might be a substantial amount of patients with the condition remain undiagnosed. The situation is made more complicate by other causes having similar symptoms: temporal arteritis, cluster headaches, acute glaucoma, meningitis, and subarachnoid hemorrhage could all have similar symptoms to migraine headache. Therefore, a non-invasive, definitive diagnosis is important for managing migraine headache.

Referring to FIG. 22A-22B, FIG. 23A-23B, and FIG. 24A-24C, the fEEGs for detection or diagnosis of migraine headache are provided in accordance with embodiments of the present disclosure. FIG. 22A-22B, FIG. 23A-23B, and FIG. 24A-24C are similar to the visual output of FIG. 10, whereby the X-axis is a scale of FM frequency and the Y-axis is a scale of AM frequency, and wherein each of the visual elements may represent the difference between an analyzed data unit and a reference data unit. In FIG. 22A-22B, the fEEGs are acquired from the mean of two EEG electrode “O1” and “O2” channels with the mean of other two EEG electrode “A1” and “A2” channels. 10 normal subjects, 10 migraine patients with aura, and 10 migraine patients without aura are analyzed in FIG. 22A-22B. The normal subjects are subjects with no migraine and are reference data units in FIG. 22A-22B; The migraine patients with or without aura are analyzed data units in FIG. 22A-22B, in accordance with the meaning of the visual elements in FIG. 10. The EEG data are collected at 256 Hz with a total length of 21 seconds. FIG. 22A shows the difference between the migraine patients with aura and the normal subjects. FIG. 22B shows the difference between the migraine patients without aura and the normal subjects. The white contour lines indicate areas wherein P-value<0.05. The results indicate that there are drastic differences between the migraine patients with aura and the migraine patients without aura. Also, there are obvious contrast between the migraine patients without aura and the normal subjects.

FIG. 23A shows the fEEG in the form of the correlation coefficient of the frequency of attack and the power level of the fEEG for the migraine with aura group. FIG. 23B shows the fEEG in the form of the correlation coefficient of the frequency of attack and the power level of the fEEG for the migraine without aura group. For the migraine without aura group, there is a clear correlation, with correlation coefficient larger than 0.9, between the frequency of head attack and the power level of the fEEG.

Referring to FIG. 24A-24C, the fEEGs on treatment response evaluation for migraine are provided in accordance with embodiments of the present disclosure. The efficacy of the two drugs for migraine treatment is tested: OnabotulinumtoxinA and topiramate. The test is conducted within 14 patients: seven patients that responds to the treatment are indicated as Responder group, and seven patients that do not respond to the treatment are indicated as Non-responder group. The EEG data is collected before, during, and post treatment, for both Responders and Non-responders. FIG. 24A shows the fEEG at “P3” channel of electrodes for the pre-med responders with contrast to the eye-closed normal subjects. There are clear differences between the normal subjects and the Responder group before treatment, with the white contour lines indicating P-value<0.05. The white contour lines in FIG. 24A have indicated the differences. An area 241 is the area of frequency modulation (FM) between 8 to 32 Hz, and amplitude modulation (AM) between 0.5 to 8 Hz. FIG. 24B shows the fEEG at station “P3” for the responders after treatment with contrast to the eye-closed normal subjects and there are no statistically significant differences. FIG. 24C shows the fEEG at station “P3” for the responders after treatment with contrast to the Responders before treatment with eyes closed, and there are no statistically significant differences.

3.5 The fEEG of Anesthesia Depth

Anesthesia is a critical and indispensable procedure to enable the practice of crucial surgery interventions. The purposes of anesthesia include hypnosis (temporary loss of conscience similar to deep sleep), amnesia (no memory), analgesia (suppression of sensation of pain) and muscle relaxation. There are several types of anesthesia: general and regional anesthesia, sedation, Spinal, epidural and caudal anesthesia and acute pain management. In surgical anesthesia, when the patient totally loss conscience, the depth of anesthesia depth becomes critical: Too light the depth could traumatize the patient undergone surgery; too deep the depth, or over dose, could result in a cessation of respiration and cardiovascular collapse that might be lethal. Consequently, there are measures to ensure the correct level of anesthesia is kept throughout the surgical procedure, including continuous Electrocardiography (ECG), continuous pulse oximetry (SpO2), blood pressure, anesthesia agent concentration, low oxygen alarm, carbon dioxide measurement, temperature, and EEG.

EEG has a critical role for assessing the depth of anesthesia. Many methods of EEG measurements for the depth of anesthesia have been proposed, including: Spectral edge frequency, Median frequency, Bispectral index (BIS), Entropy, Narcotrend index, Patient state index, Snap index, and Cerebral state index. Currently, there is no gold standard for determining anesthetic depth. Furthermore, most of the above methods of EEG measurements are based on Fourier transformation, therefore they are limited by the linear and stationary feature of Fourier transformation. A new approach on the evaluation or monitoring of anesthetic depth is needed.

FIG. 25A to FIG. 27 are the fEEGs on the evaluation or monitoring of anesthesia depth provided in accordance with embodiments of the present disclosure. FIG. 25A-25D and FIG. 26A-26D are similar to the visual outputs of FIG. 10, whereby the X-axis is a scale of FM frequency and the Y-axis is a scale of AM frequency. In FIG. 25A-FIG. 25D, the fEEGs for four different depths of anesthesia are given based on the original data for “Fp1”-“A2” channels of electrodes and classified according to different BIS values: FIG. 25A shows the fEEG of Stage I anesthesia (consciousness BIS>80), FIG. 25B shows the fEEG of Stage II anesthesia (sedation stage BIS>60 and BIS<80), FIG. 25C shows the fEEG of Stage III anesthesia (BIS>30 and BIS<60), FIG. 25D shows the fEEG of Stage IV anesthesia (BIS<30), in a course of general surgical anesthesia. In the embodiments as shown in FIG. 26A-FIG. 26D, the fEEG for four different depths of anesthesia are given based on the normalized with respect to the standard deviation for “Fp1”-“A2” channels of electrodes. The fEEGs for the Stage I (as shown in FIG. 26A), the Stage II (as shown in FIG. 26B), the Stage III (as shown in FIG. 26C), and the Stage IV (as shown in FIG. 26D), in a course of general surgical anesthesia together with the BIS indices also marked for reference.

FIG. 27 is the correlation between BIS and AM on different FM frequencies of carrier waves, in accordance with embodiments of the present disclosure. The higher the FM frequency means the higher the correlation between AM and BIS. For 45-50 Hz of FM, the correlation between AM and BIS is about 0.5-0.6. AM on β to γ bands carrier waves are significantly correlated to the BIS.

3.6 The fEEG of Sleep

There are many sleep disorders that include narcolepsy, periodic limb movement disorder (PLMD), restless leg syndrome (RLS), upper airway resistance syndrome (UARS), and the circadian rhythm sleep disorders. Amongst the most common and bothersome disorders, however, are obstructive sleep apnea and insomnia.

Insomnia is the most common sleep disorder. It is experienced by up to a third of the general population at some times, and could become a chronic and persistent condition in 10 to 15% of the population at any given time. Insomnia came in many different patterns: the patients could experience either the difficult in falling asleep initially, or waking up during sleep and could not go back to sleep again. The condition is usually reported subjectively by the patients with little quantitative monitoring of the condition. Usually treatment is light sedatives, which have all generate some controversial side effects.

Obstructive sleep apnea is another common sleep disorder. The condition is caused by the obstruction of breathing from the collapse of muscle in the airway, which is relaxed during the sleep. This could interrupt and totally stop normal breathing temporally. The lack of oxygen eventually wakes up the patient to tighten the muscle and resume breathing. In severe cases, the patient could experience this obstructive sleep apnea almost continuously throughout the night. The patient will never have a chance to get in the restful deep sleep, yet the patient is never fully awake to realize the obstruction of his sleep. As a result, the patient only experience tiredness and lack of energy without knowing the incidences of obstruction in his breathing during sleep. The diagnosis of obstructive sleep apnea is through tests in the sleep clinic or laboratory with polysomnography.

The data from the standard procedure for sleep clinic are collected with polysomnography, which consisted of the following sensors and parameter: EEG at six channels of the 10-20 system (O1, O2, C1 and C2, plus A1, A2 as references) to monitor the brain activities, Electrooculography (EOG) to monitor eye movements for REM sleep, surface Electromyography (EMG) to monitor limb movements and muscle tone, Electrocardiography for heart rate variability, Pulse oximetry for oxygen content, Respiratory effort (thoracic and abdominal) for breathing Rhyme, End tidal or transcutaneous CO 2 and Sound recordings to measure snoring for obstructive sleep apnea. Additionally, core body temperature, incident light intensity, penile tumescence, pressure, and pH at various esophageal levels may be measured. Finally, a continuous video recording of the patient throughout the night for the body movements. All these parameters are used to classify the sleep stage and sleep quality. These sensors for above parameters are so intrusive that patients would be impossible to get a natural sleep. Therefore, the tests are self-defeating procedures that would never give a true quantitative assessment of the sleep condition. A non-intrusive approach for quantitatively assess sleep condition is needed.

Referring to FIG. 28, a hypnogram of a subject is provided in accordance with embodiments of the present disclosure. FIG. 28 shows the EEG for evaluation or monitoring of sleep, wherein the X-axis is the time and the Y-axis is the stages of sleep. In FIG. 28, plateau 281a, 282a, 283a, 284a, 285a, and 286a are awake stages, and plateau 281b, 282b, 283b, 284b, 285b, and 286b are six epochs of REM sleep stages.

The fEEG would be able to separate the REM and waking state in a hypnogram shown in FIG. 28. Referring to FIG. 29A-29F, corresponding the fEEGs for each of the epochs shown in FIG. 28 are provided in accordance with embodiments of the present disclosure. The EEG signals are collected from “Fp2” and “F4” channels of electrode, and six different awake stage throughout the night are shown respectively in FIG. 29A, FIG. 29B, FIG. 29C, FIG. 29D, FIG. 29E and FIG. 29F. FIG. 29A-29F is similar to the visual output of FIG. 8, whereby the X-axis is a scale of FM frequency and the Y-axis is a scale of AM frequency. FIG. 29A corresponds to the plateau 281a in FIG. 28, and has two areas 291a and 291b representing the largest accumulated signals strengths in FIG. 29A. FIG. 29B corresponds to the plateau 282a, and has an area 292 representing the largest accumulated signal strength in FIG. 29B. FIG. 29C corresponds to plateau 283a, and has an area 293 representing the largest accumulated signal strength in FIG. 29C. FIG. 29D corresponds to plateau 284a, and has an area 294 representing the largest accumulated signal strength in FIG. 29D. FIG. 29E corresponds to plateau 285a, and has an area 295 representing the largest accumulated signal strength in FIG. 29E. FIG. 29F corresponds to plateau 286a, and has an area 296 representing the largest accumulated signal strength in FIG. 29F. The areas 291-296 are located between 16 to 32 Hz of FM and 1 to 8 Hz of AM.

In contrast to FIG. 29A-29F, the fEEGs of different REM stages are very different. Referring to FIG. 30A-30F, corresponding the fEEGs for six epochs of REM stages are provided in accordance with embodiments of the present disclosure. FIG. 30A, FIG. 30B, FIG. 30C, FIG. 30D, FIG. 30E and FIG. 30F are similar to the visual output of FIG. 29A-29F, and EEG signals are also collected from “Fp2” and “F4” channels of electrode. In FIG. 29A, there are two areas of energy concentrations: an area 291a is in 8 to 16 Hz of FM, and another area 291b of 16 to 32 Hz of FM. The one located in 8-16 Hz of FM is the typical α wave with eyes closed; the latter one located in 16-32 Hz of FM is similar to the REM. The α band, however, dissipates as the sleep progresses throughout the night. By the time the subject finally woke up, it is totally dissipated and disappeared. Meanwhile, the region of 16-32 Hz, migrate slowly upward. By the waking up time, the energy concentration is centered around 32 Hz. More significantly, while the fEEG has only energy concentrated in a relative narrow region when the subject first went into sleep, the whole the fEEG becomes much wide banded with energy in both high and low frequency range, or the brain activities are fully charged. The existence of the high (32 Hz and higher) and the low (8 Hz and low) FM range energy is a necessary condition for forming memory. This result indicates that the subject would have very low capability to memorize anything during extremely sleepy stage. Here, the effects of sleep are very clearly shown: it replenishes energy at both the high and low frequency range of the brain and makes the brain activities full again.

Referring to FIG. 31A and FIG. 31B, the fEEGs on evaluation sleep debt, monitoring sleep debt, or insomnia are provided in accordance with embodiments of the present disclosure. FIGS. 31A and 31B are similar to the visual outputs of FIG. 29A-29F. FIG. 31A comprises multiple fEEGs collected at different electrodes on an insomnia patient before sleep, and the fEEG shown in FIG. 31B are collected after the insomnia patient sleeps. The fEEGs in FIG. 31A are similar, with the largest accumulated signal strength concentrated in an area located on 16 to 32 Hz of FM. FIG. 31A are similar to the REM stage of a healthy sleeper the fEEGs in FIG. 29A-29F. Thus the fEEG could be used to determine the sleep debt.

Referring to FIG. 32A-32C, the fEEGs on evaluation or monitoring sleep are provided in accordance with embodiments of the present disclosure. Five EEG electrode arrangements are used for the measurement of FIG. 32A-32C, including Fp2-F4, F4-C4, C4-P4, P4-O2, and C4-A1. FIG. 32A-32C are similar to the visual outputs of FIG. 29A-29F. FIG. 32A shows the fEEG for REM stage, FIG. 32B shows light sleep stage, and FIG. 32C shows the fEEG for deep sleep stage. Based on these visual outputs, surface EEG is dominated by AM δ and θ bands modulating the β band carrier wave during wake and REM stage. When subjects fall asleep, surface EEG is dominated by AM δ band modulating α and β bands carrier waves. Moreover, the δ and θ bands modulating β band carrier wave becomes weaker when a subject is in deep-sleep stage.

3.7 The fEEG of Parkinson's Disease (PD)

Parkinson's disease (PD) is a long-term neuronal degenerative disorder that affects mostly the motor system. The early symptoms of PD are stooping posture, uncontrollable tremor of hands, shaking and rigidity, slowness in initiate movements. Computed tomography (CT) and conventional magnetic resonance imaging (MRI) brain scans of people with PD usually appear normal, although in more advance stages the medical imaging techniques can be used to rule out other complications. Currently, the diagnosis of PD is primarily based on medical history and neurological examination. PD patients are sometimes given levodopa to see if the symptoms are responding to the drug as a way to establish the PD cases. Even if a diagnosis is established, it is recommended that the progress of the condition be reviewed periodically in case there is other causes of the symptoms, for there are many other conditions could have similar symptoms, such as Alzheimer's disease, multiple cerebral infarction and drug-induced parkinsonism. A quantitative evidence based diagnosis is urgently needed, for in the early stage some physical therapy might offer help to retard the progress.

FIG. 33 shows the fEEG on detection or diagnosis of PD in accordance with an embodiment of the present disclosure. FIG. 33 is similar to the visual output of FIG. 10, whereby the X-axis is a scale of FM frequency and the Y-axis is a scale of AM frequency, and wherein each of the visual elements may represent the difference between an analyzed data unit and a reference data unit. In FIG. 33, EEGs of 8 PD patients are the analyzed data units and 27 normal subjects are the reference data units. The EEG data were collected from 20 electrodes at 256 Hz when eyes are closed. In FIG. 33, the fEEG data from “F3” channel of the electrodes is shown with the power level difference between PD patients and control. An area 331 with the highest power level is located in 32-128 Hz of FM and 8-64 Hz of AM. The area 331 represents statistically significant differences on t-value between EEG of the PD patients and the normal subjects.

With these results, it is possible to identify the PD patients via the fEEG. Most importantly, this quantitative result is all the more crucial for PD patients. The above method offered a quantitative measure of the variations in the brain function, which should give a more nuanced quantification of the condition if taken longitudinally.

3.8 The fEEG of Attention Deficit Hyperactivity Disorder (ADHD)

Attention deficit hyperactivity disorder (ADHD) is a neuropsychiatric condition defined as extreme short attention span and disruptive behaviors inappropriate to the subject's age. The diagnosis is based on assessments of the subject's behavior and observations of the teachers and parents. Formal diagnosis is based on the standard the Diagnostic and Statistical Manual of Mental Disorders (DSM), published by the American Psychiatric Association (APA), which are revised periodically. Additionally, International Statistical Classification of Diseases and Related Health Problems (ICD) is also used in the diagnosis.

Casual inspection of the listed symptoms seems to fit many of the immature childhood behaviors. To be certain of the ADHD condition, the subject will have to be observed over a long term: over a year in various environments such as at school and at home. Even with this caution, the diagnosis is still difficult. For example, using either the US or the international criteria would yield different classifications. The consequence of the lack of diagnostic certainty is dire indeed, for all the sufferers are young children in the development stage. There is a need for a definitive diagnostic approach for ADHD.

An early and definitive diagnosis of ADHD can be achieved by the fEEGs. The fEEG is a non-intrusive approach and intuitive approach for diagnosing ADHD. One or more specific channels of electrode can be selected, and EEG signals can be collected from selected electrodes over a time period. Individuals with or without ADHD can be the subjects of the fEEG, and specific the fEEG patterns of these groups can be identified and summarized for the diagnosis of ADHD.

3.9 The fEEG of Drug Addiction

Drug addiction is a brain disorder that involves compulsive actions to obtain rewarding stimuli from various form of substance such as alcoholism, amphetamine addiction, cocaine addiction, nicotine addiction, opiate addiction, food addiction, gambling addiction, and sexual addiction. The lack of inhibitory control in drug addiction is similar to Attention Deficit Hyperactivity Disorder (ADHD).

An observation on EEGs may be helpful for mitigating the drug addiction. EEG signals can be combined to form the fEEG visual outputs, and specific the fEEG patterns may be identified among the subjects with drug addiction.

4. Clinical Applications of Functional Electroencephalotopography (fEEToPG)

The fEEToPG is capable of representing dynamic profiles of brain activities with high spatial and temporary resolutions. In comparison with functional magnetic resonance imaging (fMRI), despite the fact that EEG has less spatial resolution, the fEEToPG is able to reflect the underlying brain activities via particular modulation methods for the EEG signals. the fEEToPG is helpful to identify the major reaction areas in the human brain in response to environmental stimuli. Moreover, there are three major advantages of using the fEEToPG; they are: 1) The cost of EEG is much cheaper than that of a fMRI and the measurement of EEG is much easier to be carried out than that of fMRI; the patient is generally more compliant to the EEG system, and it would be possible to have a wearable EEG system at home; 2) The multi-channel EEG in the fEEToPG may overcome the spatial resolution issue and offer temporal resolution to identify cognitive functions; it can be used to identify the brain regions involved in such cognitive functions, or the precise time of the effects from the experimental stimulation; 3) Results of the fEEToPG offer not only information about brain regions involved in cognitive functions, but also the underlying mechanisms of dynamic modulation in amplitude modulation (AM) and frequency modulation (FM) and their corresponding carrier components.

4.1 The fEEToPG of Alzheimer's Disease (AD)

FIG. 34A-FIG. 36D are visual outputs of the fEEToPGs on detection or diagnosis of Alzheimer's disease (AD), in accordance with embodiments of the present disclosure. In one embodiment, a cross sectional study is designed for healthy subjects and AD patients of different CDRs (Clinical Dementia Rating and Mini-Mental State Examination). 19 patients with mild cognition impairment and 55 patients diagnosed with AD with different CDR (22 patients are CDR1, 21 patients are CDR2, and 14 patients are CDR3) are classified as AD patients. The EEG signals are acquired in a ten-second duration with a sampling rate of 256 Hz.

FIG. 34A-34B are visual outputs of the fEEToPG comprising a plurality of topographies of the brain, in accordance with an embodiment of the present disclosure. FIG. 34A-34B are similar to the visual output in FIG. 12 However, FIG. 12 is the visual output of the fEEToPG generated directly from brain electrical activities collected over a period of time, but FIG. 34A-34B are the results of comparing two different groups of the fEEToPG. The visual elements in FIG. 34A-34B are defined by the X-axis of AM frequency and the Y-axis of FM frequency. For instance, a visual element 341 is located on 2-4 Hz of AM and 2-4 Hz of FM. The visual element 341 further comprises a plurality of probabilities for quantifying the statistical significance between the two different groups of the fEEToPG. The two different groups of the fEEToPG used for comparison in FIG. 34A are the healthy subjects and the mild cognitive impairment (MCI) patients, and CDR1 patients and MCI patients are compared in FIG. 34B. Each of the visual element has a boundary defining a two-dimensional anatomical graph of the brain.

FIG. 34A demonstrates clear differences between the healthy subjects and the MCI group. FIG. 34A demonstrates higher t-score at 1-8 Hz of FM and lower t-score at an area of 2-8 Hz of AM and 16-128 Hz of FM when the MCI group is compared with the healthy subject group. In FIG. 31B, the difference between the CDR1 patients and the MCI patients is significant (with two tailed p value less than 0.05) again for all over the brain. Furthermore, the t-score is especially higher in an area of 4-8 Hz of FM and 0.5-4 Hz of AM, indicating the similar shift of energy to lower FM region.

It should also be pointed out that the conventional ‘topography’ of the brain is not useful at all, for they integrated out of all the details of AM frequency modulations and presenting the result only on with the frequency variations. This pattern of results in FIG. 34A-34C have demonstrated the fEEToPG can differentiate MCI from normal health subjects objectively and quantitatively.

FIG. 35A-35C are visual outputs of the fEEToPG comprising a plurality of topographies of the brain, in accordance with an embodiment of the present disclosure. FIG. 35A-35C are similar to the visual outputs of FIG. 34A-34B, and both groups of figures demonstrate comparisons between different groups. FIG. 35A-35C are longitudinal studies: a group of AD patients with different CDR scores are recruited. The fEEToPG of CDR1 patients are compared with the fEEToPG of CDR0.5 (MCI) patients in FIG. 35A, CDR2 patients are compared with CDR1 patients in FIG. 35B, CDR2 patients are compared with CDR3 patients in FIG. 35C. FIG. 35A demonstrates significant difference (with a two tailed p value less than 0.5 between CDR1 patients and CDR0.5 (MCI) patients. The t-score in FIG. 35A is higher in an area of 2-8 Hz of FM and 0.5-4 Hz of AM. FIG. 35B demonstrates significant difference between CDR2 patients and CDR1 patients, albeit at a lower level of significance. The t-score in FIG. 35B is higher in an area of 8-32 Hz of FM and 0.5-4 Hz of AM. FIG. 35C demonstrate less differences than FIG. 35A or FIG. 35B. It should be pointed out that the initial change from MCI to CDR1 as shown in FIG. 35A is much more important than the changes from CDR2 to CDR3 as shown in FIG. 35C.

FIG. 35D is a receiver operating curve (ROC) for illustrating the utility of the fEEToPG, in accordance with embodiments of the present disclosure. In the embodiment, it is possible to set a criterion containing the most physiologically relevant channels of electrodes—“Fz”, “Cz”, “Pz”, “T3”, and “T5” for AD in a certain frequency band (range: 4-32 Hz) modulated by AM (range: 0.5-2 Hz) to calculate the sensitivity and specificity (right-lower inset). The sensitivity and specificity of this chosen criterion have approached 80% and 90% respectively, which have met the consensus criterion suggested by the Ronald and Nancy Reagan Research Institute of the Alzheimer's Association.

FIG. 36A-FIG. 36D are visual outputs of the fEEG and the fEEToPG on evaluation the progression of disease status of AD, in accordance with embodiments of the present disclosure. In order to further establish the validity of the fEEToPG method, a correlation study is conducted, in which the inter-comparisons between the power level of the fEEToPG and the CDR scores and the inter-comparisons between the power level of the fEEToPG and MMSE (Mini-Mental State Examination) scores are correlated. FIG. 36A shows the correlation map of an electrode between the fEEG and the CDR scores. The inter-comparisons between the power level of the fEEToPG and CDR scores are shown in FIG. 36B. FIG. 36C shows the correlation map of an electrode between the fEEGs and the MMSE scores. The inter-comparisons between the power level of the fEEToPG and MMSE scores are shown in FIG. 36D. The high correlation coefficients are reassuring that one can transition the previous subjective behavior based evaluation of dementia and AD cases to evidence based fEEToPG diagnosis. The more objective quantitative score for AD is available based on the fEEG and the fEEToPG.

4.2 The fEEToPG of Huntington's Disease (HD)

FIG. 37A-FIG. 37C shows the visual outputs of the fEEToPG on detection or diagnosis of Huntington's disease (HD), in accordance with embodiments of the present disclosure. FIG. 37A-37C are similar to the visual output of FIG. 34A-34C. In the embodiment, 15 HD patients and five pre-HD carriers and 27 non-HD subjects have been recruited for the study. The fEEToPG can offer narrow-band EEG time frequency information over the whole scalp, and the difference among the three groups is highlighted. The fEEToPG of HD patients are compared with non-HD subjects in FIG. 37A. The fEEToPG of HD patients are compared with pre-HD carriers in FIG. 37B. The fEEToPG of HD patients are compared with pre-HD carriers in FIG. 37C. In FIG. 37A, a visual output of the fEEToPG indicates that there are significant differences between the pre-HD carriers and non-HD subjects in a region of 2-8 Hz of AM and 32-128 Hz of FM, and a larger region of 1-16 Hz of AM and 32-64 Hz of FM. In FIG. 37B, a visual output of the fEEToPG indicates that there are significantly differences between HD patients and pre-HD carriers in a region of 0.5-64 Hz of AM and 32-128 Hz of FM. In FIG. 37C, there are no statistically significant differences between pre-HD carriers and HD patients.

As shown in FIG. 38, the current data indicate that the pre-HD burden score is negatively correlated with the α/γ power ratio index (r=−0.9389, p=0.0055), and HD/Pre-HD (Mini-Mental State Examination) is positively correlated with the α/γ power ratios (r=0.3862, p=0.0379, one-tail). These results suggest that this index can be a potential EEG biomarker for predicting the onset of HD and monitoring the progression of HD. The source localization data implies that the sources are mostly from the occipital/parietal lobe for the α band and from the whole brain for γ band. This set of information can also be used as indices for potential intervention to be address in a separate patent.

4.2 The fEEToPG of Depression

FIG. 39A-39C are visual outputs of the fEEToPG on detection or diagnosis of depression or mood disorder, in accordance with embodiments of the present disclosure. FIG. 39A-39C are similar to the visual output of FIG. 12. FIG. 39A shows the fEEToPG of the healthy subjects when eyes are closed, and FIG. 39B shows the fEEToPG of the depression patients when eyes are closed. FIG. 39C shows a comparison of the fEEToPGs between the depression patient and healthy subjects, both with eyes closed. In FIG. 36C, the differences are widely spread from 8 to 128 Hz of AM, and also on all Hz of FM, wherein the most significant differences (with p value less than 0.002) are located in a region of 16-32 Hz FM. This indicates that, for the depression patients, their brain is never calm but stay in a highly agitated states.

4.3 The fEEToPG of Migraine Headache

FIG. 40A-FIG. 40E are visual outputs of the fEEToPGs on detection or diagnosis of migraine and treatment responses thereof, in accordance with embodiments of the present disclosure. FIG. 40A-40E are similar to the visual outputs of FIG. 12. FIG. 40A shows a comparison of the fEEToPGs between the pre-med responders and healthy subjects, with both of their eyes closed. FIG. 40B shows a comparison of the fEEToPGs between the post-med responders and healthy subjects, with both groups' eyes closed. FIG. 40C shows a comparison of the fEEToPG between the post-med responders and pre-med responders, with both groups' eyes closed. In FIG. 40A, the visual output of the fEEToPG shows statistically significant difference in a region 401 covering 2-32 Hz of frequency modulation (FM) and 0.25-4 Hz of amplitude modulation (AM), where the migraine patients showed a drastic deficit of power, with the white circles indicating the detection units for having a P-value<0.05. In FIG. 40B, the visual output of the fEEToPG shows statistically significant differences in a region 402 covering 64-128 Hz of FM, for the post-med responders and the healthy subjects with eyes closed. In FIG. 40C, the visual output of the fEEToPG shows statistically significant differences in a region 403 covering 2-32 Hz of FM and 0.25-4 Hz of AM, for the post-med responders and the pre-med responders with eyes closed. The embodiment shows showing a general elevation of brain wave power in the pre-treatment depressed area pushing the overall pattern to the normal level as in the health control. FIG. 40D shows a comparison of the fEEToPGs between the pre-med non-responders and the healthy subjects, with both groups' eyes closed. FIG. 40E shows a comparison of the fEEToPGs between the post-med non-responders and the healthy subjects, with both groups' eyes closed. FIGS. 40D and 40E show the lack of differences between these groups, except for those topographies at higher FM frequencies and AM frequencies. This means the brain waves measured by EEG of the non-responders is similar to the healthy subjects, and the treatment could not change the patterns.

4.3 The fEEToPG of Parkinson's Disease (PD)

FIG. 41 is a visual output of the fEEToPG on detection or diagnosis of Parkinson's disease (PD) in accordance with an embodiment of the present disclosure. FIG. 41 is similar to the visual output of FIG. 12, and is a comparison between the fEEToPG of 8 PD patients and 27 healthy subjects, when both groups' eyes are closed. In the embodiment, the fEEGToPG shows statistically significant differences in the region 411 covering 32-128 Hz of FM and 16-32 Hz of AM. There is also statistically significant difference in a large region 412 covering 64-128 Hz of FM and 0-64 Hz of AM.

4.4 The fEEToPG of Attention Deficit Hyperactivity Disorder (ADHD)

FIG. 42A and FIG. 42B are visual outputs of the fEEToPGs on detection or diagnosis of attention deficit hyperactivity disorder (ADHD), in accordance with embodiments of the present disclosure. FIG. 42A and FIG. 42B are similar to the visual output of FIG. 12. FIG. 42A is a comparison of the fEEToPGs between eyes opened and eyes closed conditions in ADHD patients. In FIG. 42A, statistically significant difference (with P-value<0.05) shows up over a narrow region of 0 to a band (4-16 Hz of FM). FIG. 42B is a comparison of the fEEToPGs between eyes opened and eyes closed conditions in healthy subjects. The pattern in the fEEToPGs of FIG. 42A is very different from the results of the healthy subjects as shown in FIG. 42B, wherein the major difference between FIG. 42A and FIG. 42B is over a to β bands (8-32 Hz of FM). ADHD may be diagnosed based on the relative strength in the narrow region of θ to α band (4-16 Hz of FM).

5. Clinical Applications of the fEEToMG

5.1 The fEEToMG of Alzheimer's Disease (AD)

FIG. 43A-FIG. 44B are visual outputs of the fEEToMG for evaluating the progression of Alzheimer's disease (AD) measured by CDR and MMSE (Mini-Mental State Examination), in accordance with embodiments of the present disclosure. In the embodiment, areas of significant correlation are identified. FIG. 43A shows the lateral view of the fEEToMG correlated to CDR score with significant contrast on a region 431 and another region 432. FIG. 43B shows the medial view of the fEEToMG correlated to CDR score with significant contrast on a region 433 and another region 434. FIG. 44A shows the lateral view of the fEEToMG correlated to MMSE score with significant contrast on a region 441. FIG. 44B shows the medial view of the fEEToMG correlated to MMSE score with significant contrast on a region 442. Of particular importance is that the correlation also shows a region of statistically significant difference with P-value<0.05 along 8-128 Hz of FM and 4-8 Hz of AM.

5.2 The fEEToMG of Huntington's Disease (HD)

FIG. 45A, FIG. 45B and FIG. 45C are visual outputs of the fEEToMGs on detection or diagnosis of Huntington's disease (HD), in accordance with embodiments of the present disclosure. In the embodiment, the fEEToMG is obtained on the dyadic scale and calculating the ratio of 8-16 Hz band power and 32-64 Hz band power in the posterior parieto-temporal-occipital electrodes. FIG. 45A-FIG. 45C are similar to the visual output of FIG. 16. FIG. 45A is a comparison of the fEEToMGs between Pre-HD carriers and healthy subjects, with significant contrast on a region 451. FIG. 45B is a comparison of the fEEToMGs between HD patients and the healthy subjects, with significant contrast on a region 452. FIG. 45C is a comparison of the fEEToMGs between Pre-HD carriers and HD patients, with significant contrasts on a region 453 and a region 454. As shown in FIG. 45A and FIG. 45B, the results indicate that both Pre-HD carriers and HD patients have significantly smaller power ratio than the healthy subjects. In FIG. 45C, the degree of difference also occurred between HD patients and Pre-HD carriers (HD vs the healthy subjects, P-value<0.001; Pre-HD carriers vs the healthy subjects, P-value=0.02; HD patients vs Pre-HD carriers, P-value<0.05). These results suggest that this index can be a potential biomarker for predicting the onset of HD and monitoring the progression of HD.

5.3 The fEEToMG of Migraine Headache

FIG. 46A-FIG. 46E are visual outputs of the fEEToMGs on detection or diagnosis of migraine and the evaluation of treatment response thereof, in accordance with embodiments of the present disclosure. FIG. 46A-FIG. 46E are similar to the visual output of FIG. 16. FIG. 46A is a comparison of the fEEToMGs between pre-med responders and healthy subjects, with both groups' eyes closed. FIG. 46B is a comparison of the fEEToMGs between post-med responders and the healthy subjects, with both groups' eyes closed. FIG. 46C is a comparison of the fEEToMGs between the post-med responders and the pre-med responders, with both groups' eyes closed. The embodiment shows showing a general elevation of brain wave power in the pre-treatment depressed area pushing the overall pattern to the normal level as in the health control. FIG. 46D is a comparison of the fEEToMGs between the pre-med non-responders and the healthy subjects, with both groups' eyes closed. FIG. 46E is a comparison of the fEEToMGs between the post-med non-responders and the healthy subjects, with both groups' eyes closed. FIG. 46D and FIG. 46E show a lack of difference, except in regions of higher FM and higher AM frequencies.

5.4 The fEEToMG of Parkinson's Disease (HD)

FIG. 47 and FIG. 48 are the fEEToMGs on detection or diagnosis of Parkinson's disease (PD), in accordance with embodiments of the present disclosure. FIG. 47 is a comparison of the fEEToMGs between eight PD patients and 27 non-PD subjects, with both groups'eyes closed. In the embodiment as shown in FIG. 47, the fEEToMG shows prominent difference in a tomography 471 of 64-128 Hz of FM and 16-32 Hz of AM. An enlarged view is shown in FIG. 48, wherein the statistically significant difference significances of tomography 471 Hz are highlighted. With these results, it is possible to quantify the disease progress in PD patients. This quantitative result is crucial for PD patients, for the conventional diagnosis needs periodic re-examination and evaluation to confirm the diagnosis, as well as measuring the progress of the disease. Because there is no objective standard, such evaluation is difficult, if possible at all. The present disclosure offers a quantitative measure for the variations in the brain function, which should give a more nuanced quantification of the condition if taken longitudinally.

The embodiments shown and described above are only examples. Many details are often found in the art such as the other features of a circuit board assembly. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including the full extent established by the broad general meaning of the terms used in the claims. It will therefore be appreciated that the embodiments described above may be modified within the scope of the claims.

What is claimed is:

1. A non-transitory computer program product embodied in a computer-readable medium and, when executed by one or more analysis modules, providing a visual output for presenting electrical activities of at least one brain, comprising:
a first axis representing frequency modulation (FM);
a second axis representing amplitude modulation (AM); and
a plurality of visual elements, each of the visual elements being defined by the first axis and the second axis, and each of the visual elements comprising an accumulated signal strength and a plurality of analyzed data units collected over a time period,
wherein each of the analyzed data units comprises a first coordinate, a second coordinate, and a signal strength value, the first coordinate is an argument of a FM function from a transformation on a primary intrinsic mode function (IMF), the second coordinate is an argument of an AM function from a transformation on a secondary IMF, each of the primary IMF is generated from an empirical mode decomposition (EMD) of a plurality of electrical activity signals, and each of the secondary IMF is generated from an EMD of the primary IMF, and the accumulated signal strength is an integral of the signal strength values of the analyzed data units.

2. The non-transitory computer program product of claim 1, wherein the first axis is a logarithmic scale of frequency modulation (FM), the second axis is a logarithmic scale of amplitude modulation (AM), the first coordinate is a logarithmic value of the argument of the FM function, and the second coordinate is a logarithmic value of the argument of the AM function.

3. The non-transitory computer program product of claim 1, wherein the analyzed data units are generated from electroencephalograhy (EEG), magnetoencephalography (MEG), or electrocorticography (ECoG).

4. The non-transitory computer program product of claim 1, wherein the visual elements are functional electroencephalotopography (fEEToPG) or functional electroencephalotomography (fEEToMG).

5. The non-transitory computer program product of claim 4, wherein each of the visual elements further comprises a boundary defining an anatomical graph, and the anatomical graph is a two-dimensional graph of the brain when the visual elements are fEEToPG.

6. The non-transitory computer program product of claim 5, wherein the visual element further comprises one or more detection unit IDs in the boundary, and each of the detection unit IDs has one of the accumulated signal strengths.

7. The non-transitory computer program product of claim 6, wherein the visual element further comprises a plurality of intermediate areas within the boundary and between the detection unit IDs, and each of the intermediate areas has a modeled accumulated signal strength.

8. The non-transitory computer program product of claim 4, wherein each of the visual elements further comprises a boundary defining an anatomical graph, and the anatomical graph is a three-dimensional graph of the brain when the visual elements are fEEToMG.

9. The non-transitory computer program product of claim 8, wherein the visual element further comprises one or more detection unit IDs in the boundary, and each of the detection unit IDs has one of the accumulated signal strengths.

10. The non-transitory computer program product of claim 9, wherein the visual element further comprises a plurality of intermediate areas within the boundary and between the detection unit IDs, and each of the intermediate area has a modeled accumulated signal strength.

11. The non-transitory computer program product of claim 1, wherein the accumulated signal strength in each of the visual elements is indicated by different colors, grayscales, dot densities, contour lines, or screentones.

12. A non-transitory computer program product embodied in a computer-readable medium and, when executed by one or more analysis modules, providing statistical significance between at least two visual outputs, comprising:
a first axis representing frequency modulation (FM);
a second axis representing amplitude modulation (AM); and
a plurality of visual elements, each of the visual elements being defined by the first axis and the second axis, and each of the visual elements comprising a probability for quantifying the statistical significance between other visual outputs,
wherein each of the visual outputs comprises a plurality of analyzed data units collected over a time period, each of the analyzed data units comprises a first coordinate, a second coordinate, and a signal strength value, the first coordinate is an argument of a FM function from a transformation on a primary intrinsic mode function (IMF), the second coordinate is an argument of an AM function from a transformation on a secondary IMF, each of the primary IMF is generated from an empirical mode decomposition (EMD) of a plurality of electrical activity signals, and each of the secondary IMF is generated from an EMD of the primary IMF, and the accumulated signal strength is an integral of the signal strength values of the analyzed data units.

13. The non-transitory computer program product of claim 12, wherein the probability for quantifying the statistical significance is a P-value.

14. The non-transitory computer program product of claim 12, wherein the visual element further comprises a boundary defining an anatomical graph, and the anatomical graph is a two dimenstional graph of the brain when the visual elements are fEEToPG.

15. The non-transitory computer program product of claim 14, wherein the probability for quantifying the statistical significance is a P-value.

16. The non-transitory computer program product of claim 12, each of the visual element further comprising a boundary defining an anatomical graph, and the anatomical graph is a three-dimensional graph of the brain when the visual elements are fEEToMG.

17. The non-transitory computer program product of claim 16, wherein the probability for quantifying the statistical significance is a P-value.

18. A system for analyzing electrical activities of at least one brain, comprising:
a detection module for detecting the electrical activities of the brain;
a transmission module for receiving electrical activity signals from the detection module and delivering the electrical activity signals to the analysis module;
an analysis module for generating a plurality of analyzed data sets from the electrical activity signals, and
a visual output module for rendering a visual output space according to the analyzed data sets generated by the analysis module, and displaying a visual output,
wherein the visual output comprises a first axis representing frequency modulation (FM), a second axis representing amplitude modulation (AM), and a plurality of visual elements defined by the first axis and the second axis, and each of the visual elements comprises an accumulated signal strength and the analyzed data sets, and each of the analyzed data sets comprises a plurality of analyzed data units collected over a time period, each of the analyzed data units comprises a first coordinate, a second coordinate, and a signal strength value, the first coordinate is an argument of a FM function and the second coordinate is an argument of a AM function, and the accumulated signal strength is an integral of the signal strength values of the analyzed data units.

19. The system of claim 18, wherein the first axis is a logarithmic scale of frequency modulation (FM), the second axis is a logarithmic scale of amplitude modulation (AM), the first coordinate is a logarithmic value of the argument of the FM function, and the second coordinate is a logarithmic value of the argument of the AM function.

20. The system of claim 18, further comprising a non-transitory computer program product for presenting the electrical activities of the brain, wherein the computer program product comprises sets of instructions that, when executed by the analysis module, causes the analysis module to perform actions comprising:
1) performing empirical model decomposition (EMD) on the electrical activity signals to generate a set of primary intrinsic mode functions (IMFs);
2) performing the EMD on the set of primary IMFs to generate a set of secondary IMFs;
3) performing transformations on the set of primary IMFs to generate FM functions and on the set of secondary IMFs to generate AM functions; and
4) combining the AM functions and the FM functions to generate a plurality of the analyzed data sets.

21. The system of claim 18, wherein the analyzed data units are generated from electroencephalography (EEG), magnetoencephalography (MEG), or electrocorticography (ECoG).

22. The system of claim 18, wherein the visual elements are electroencephalotopography (fEEToPG) or functional electroencephalotomography (fEEToMG).

23. The system of claim 22, wherein each of the visual element further comprising a boundary defining an anatomical graph, and the anatomical graph is a two-dimensional graph of the brain when the visual elements are fEEToPG

24. The system of claim 22, wherein the visual element further comprises one or more detection unit IDs in the boundary, and each of the detection unit ID has one of the accumulated signal strength.

25. The system of claim 24, wherein the visual element further comprises a plurality of intermediate areas within the boundary and between the detection unit IDs, and each of the intermediate area has a modeled accumulated signal strength.

26. The system of claim 22, wherein each of the visual element further comprises a boundary defining an anatomical graph, and the anatomical graph is a three-dimensional graph of the brain when the visual elements are fEEToMG.

27. The system of claim 26, wherein the visual element further comprises one or more detection unit IDs in the boundary, and each of the detection unit ID has one of the accumulated signal strength.

28. The system of claim 27, wherein the visual element further comprises a plurality of intermediate areas within the boundary and between the detection unit IDs, and each of the intermediate area has a modeled accumulated signal strength.

29. The system of claim 18, wherein the accumulated signal strength in each of the visual elements is indicated by different colors, grayscales, dot densities, contour lines, or screentones.

30. A system for analyzing electrical activities of at least one brain, comprising:
an analysis module for generating a set of probabilities for quantifying statistical significance between at least two visual outputs, wherein each of the visual outputs comprises a plurality of analyzed data units collected over a time period, each of the analyzed data units comprises a first coordinate, a second coordinate, and a signal strength value, the first coordinate is an argument of a frequency modulation (FM) function from a transformation on a primary intrinsic mode function (IMF), the second coordinate is an argument of an amplitude modulation (AM) function from a transformation on a secondary IMF, each of the primary IMF is generated from an empirical mode decomposition (EMD) of a plurality of electrical activity signals, and each of the secondary IMF is generated from an EMD of the primary IMF, and the accumulated signal strength is an integral of the signal strength values of the analyzed data units; and
a visual output module for rendering a visual output space according to the set of probabilities, and displaying a visual output, wherein the visual output comprises a first axis representing FM, a second axis representing AM, and a plurality of visual elements defined by the first axis and the second axis, and each of the visual elements comprises a probability for quantifying the statistical significance between other visual outputs.

31. The system of claim 30, wherein the probability for quantifying statistical significance is a P-value.

32. The system of claim 30, wherein each of the visual elements further comprises a boundary defining an anatomical graph, and the anatomical graph is a two-dimensional graph of the grain when the visual elements are fEEToPG.

33. The system of claim 32, wherein the probability for quantifying statistical significance is a P-value.

34. The system of claim 30, wherein each of the visual elements further comprises a boundary defining an anatomical graph, and the anatomical graph is a three-dimensional graph of the brain when the visual elements are fEEToMG.

35. The system of claim 34, wherein the probability for quantifying statistical significance is a P-value.
